(19) United States
(12) Patent Application Publication
(10) Pub. No.: US 2026/0347423 A1
(43) Pub. Date: Nov. 5, 2026

(54) COMPOSITIONS AND METHODS FOR PRODUCING IL-16

(71) Applicant: ImmunityBio, Inc., San Diego, CA (US)

(72) Inventors: Patrick SOON-SHIONG, Los Angeles, CA (US); Laurent BOISSEL, Woburn, MA (US); Hans KLINGEMANN, Woburn, MA (US); Himani CHINNAPEN, Quincy, MA (US); Barry SIMON, San Diego, CA (US); Wenjie HU, San Diego, CA (US)

(21) Appl. No.: 19/182,320

(22) Filed: Apr. 17, 2025

Related U.S. Application Data
(60) Provisional application No. 63/635,805, filed on Apr. 18, 2024.
(60) Provisional application No. 63/686,966, filed on Aug. 26, 2024.

(51) Int. Cl. C07K 14/54; A61K 35/17; A61K 38/00; A61P 35/00; C07K 14/47; C12N 5/0783; C12N 9/64

(52) U.S. Cl. CPC C07K 14/5446; A61K 35/17; A61P 35/00; C07K 14/47; C12N 5/0646; C12N 9/6467; C12Y 304/21078; C12Y 304/21079; A61K 38/00; C12N 2510/02

(57) ABSTRACT

An NK cell lysate is provided herein that includes a salt solution, a cytotoxic protein, and IL-16 endogenously produced by NK cells. Also provided is a composition including the NK cell lysate and optionally NK cell exosomes. A method of preparing the NK cell lysate and composition is provided herein, including lysing the NK cells to produce a lysate and removing intact NK cells, NK cell debris, and NK cell genomic DNA from the lysate, as well as a method for generating IL-16 from NK cells. Also provided herein are methods for stimulating an immune response, preventing, reducing the occurrence of, and/or treating an infection; preventing, reducing the occurrence of, and/or treating cancer; and initiating, enhancing, or prolonging an anti-tumor response. Also provided is a combination therapy including administering the NK cell lysate or composition and a therapeutically active agent, and a vaccine including the NK cell lysate or composition.

### CROSS-REFERENCES TO RELATED APPLICATIONS

This patent application claims the benefit of U.S. Provisional Application No. 63/635,805 filed on 18 Apr. 2024, and U.S. Provisional Application No. 63/686,966 filed on 26 Aug. 2024, the entire contents of which are hereby incorporated by reference.

### SEQUENCE LISTING

The patent application contains a Sequence Listing which has been submitted electronically in XML format and is hereby incorporated by reference in its entirety. Said XML copy, created on Apr. 14, 2025, is named 104066-1498933-10100US_SL.xml and is 6,101 bytes in size.

### FIELD

This disclosure generally relates to compositions and methods for producing IL-16 from natural killer (NK) cells. Also disclosed herein are methods for treating disease using an NK cell lysate described herein.

### BACKGROUND

This section provides background information related to the present disclosure which is not necessarily prior art.

Natural killer (NK) cells are a type of cytotoxic lymphocyte and belong to the family of innate lymphoid cells (ILCs). NK cells provide rapid responses to virus-infected cells and other pathogens and respond to tumor formation. The human NK-92 (aNK) cell line has become the most widely used NK cell line for translational research on NK cells. NK-92 has also been administered to cancer patients in several phase 1 and phase 2 studies. The original NK-92 cell line has been genetically modified to grow independent of IL-2, express a high affinity Fc-receptor, and also express various chimeric antigen receptors (CARs).

Although NK cells are widely used in clinical applications, the NK-92 cell line has been generated from blood cells of a patient with lymphoma and can induce an allogeneic cell response in a recipient, wherein a potent immune response may result in allograft rejection. Current methods to reduce the risk of an allogeneic response include irradiation of NK cells prior to infusion, which adds a layer of complexity to treatment logistics. Despite efforts to produce NK cells that have a reduced allogeneic response, there is a need in the art for new and improved compositions and methods for employing their cytotoxic effects.

There is also a need to produce IL-16 for clinical use. IL-16 is a cytokine that can be used as a biomarker in many cancers and inflammatory diseases and has many properties such as regulating T cell activation and proliferation, inducing chemotaxis in CD4+ T cells, and inhibiting HIV replication. IL-16 also undergoes proteolytic processing, resulting in two functional products. IL-16 is predominantly produced by CD4+ and CD8+ cells, although IL-16 is also known to be generated by monocytes/macrophages, dendritic cells (DCs), mast cells, and fibroblasts. IL-16 is still a relatively uncharacterized cytokine, and current methods to produce IL-16 include subjecting T cells to antigen or mitogen exposure, which can induce IL-16 synthesis. IL-16 can also be recombinantly produced using E. coli. However, these methods may be time consuming, and developing a simple, fast way to produce IL-16 for therapeutic use would be beneficial.

### SUMMARY

This section provides a general summary of the disclosure and is not a comprehensive disclosure of its full scope or all of its features.

In some aspects, the present disclosure provides a natural killer (NK) cell lysate including a salt solution, a cytotoxic protein, and a cytokine. In some aspects, the cytokine is IL-16. In certain aspects, the cytokine is an active form of IL-16. In some aspects, the IL-16 is endogenously produced by NK cells. In some aspects, the cytotoxic protein may be granzyme A (GzmA), granzyme B (GzmB), granzyme H (GzmH), granzyme K (GzmK), granzyme M (GzmM), gasdermin D (GSDMD), perforin 1 (PRF1), or granulysin (GNLY). In some aspects, the NK cell lysate may include one or more additional cytotoxic proteins and/or cytokines. In some aspects, the NK cell lysate may include one or more additional cytokines, agonist derivatives thereof, or fusion constructs comprised thereof. The additional cytokine, agonist derivative thereof, or fusion construct comprised thereof may be IL-1α, IL-1β, IL-2, IL-4, IL-5, IL-6, IL-7, IL-10, IL-12p40, IL-12p70, IL-13, IL-15, IL-17, IL-18, INF-γ, GM-CSF, TNF-α, TNF-β, VEGF, IL-8, Eotaxin-3, MCP-1, MCP-4, MDC, MIP-1α, MIP-1β, or TARC. In certain aspects, the cytokine(s) and cytotoxic protein(s) may be endogenously or recombinantly produced by NK cells. In other aspects, the cytokine(s) and cytotoxic protein(s) may be exogenously added before or after lysing the NK cells. In some aspects, the NK cells may be (i.e., may be derived from) NK-92/aNK cells, haNK cells, t-haNK cells, primary NK cells, KHYG-1 cells, or variants thereof.

The present disclosure also provides a composition including the NK cell lysate described herein. Also described herein is a composition including NK cell exosomes and NK cell lysate. In some aspects, the exosomes may be derived from primary NK cells, where the NK cells are expanded with IL-2, IL-12, IL-15, IL-18, or a combination thereof, thereby producing memory-like cytokine enhanced NK cells (m-ceNK). The composition may also include a pharmaceutically acceptable carrier. In some aspects, the composition may be used in preventing or treating cancer; initiating, enhancing, or prolonging an anti-tumor response; or stimulating an immune response. The present disclosure also provides a vaccine including the NK cell lysate or composition described herein.

In yet other aspects, the present disclosure provides a method of preparing the NK cell lysate described herein. In some aspects, the method includes lysing the NK cells to produce a lysate and removing intact NK cells, NK cell debris, and NK cell genomic DNA from the lysate. In some aspects, lysing the NK cells includes one or more cycles of sonicating the NK cells or one or more cycles of freezing and thawing the NK cells. In further aspects, the method may include substantially removing MHC class I and/or MHC class II from the lysate. In further aspects, the method may include expanding the NK cells in culture medium prior to lysis. The disclosure also provides a method of producing IL-16 from NK cells. In some aspects, the method includes isolating IL-16 from the NK cell lysate.

Also provided herein is a method of preparing a composition, for example for use in the treatment of cancer. In some aspects, the method includes expanding primary human NK cells in a cell culture including IL-15 or an agonist derivative thereof, IL-12 or an agonist derivative thereof, and IL-18 or an agonist derivative thereof, thereby producing memory-like cytokine enhanced NK cells (m-ceNK). The method may also include collecting exosomes from the supernatant of m-ceNK cells as well as lysing the m-ceNK cells to produce a lysate. Lysing the m-ceNK cells may include one or more cycles of sonicating the m-ceNK cells or one or more cycles of freezing and thawing the m-ceNK cells. The method also may include removing intact NK cells, NK cell debris, and NK cell genomic DNA from the lysate. The method includes formulating an effective amount of the exosomes and an effective amount of the lysate for administration to a human subject in need thereof.

The present disclosure also provides methods for stimulating an immune response; preventing and/or treating an infection; preventing and/or treating cancer; and initiating, enhancing, or prolonging an anti-tumor response. In some aspects, the methods may include administering to a subject an effective amount of the NK cell lysate or composition including the NK cell lysate. In certain aspects, the NK cell lysate or composition may be administered via intravenous injection, inhalation, intratumoral injection, or injection into an infected abscess. In certain aspects, the NK cell lysate or composition may induce tumor cell death and/or stop tumor cell proliferation. In some aspects, the NK cell lysate or composition may provide protection against tumor reoccurrence.

Further aspects include a combination therapy for preventing and/or treating cancer. The combination therapy may include administering a first component including the NK cell lysate or composition described herein along with a second component including a therapeutically active agent. In some aspects, the therapeutically active agent may be a chemotherapeutic agent, a cell therapy, a targeted drug, or an immunomodulator.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings described herein are for illustrative purposes only of selected aspects and not all possible implementations, and are not intended to limit the scope of the present disclosure.

FIG. 1 is a flow cytometric/fluorescence-activated cell sorting (FACS) cytotoxicity assay showing the effect of aNK (NK-92) cell lysate on K562 cells.

FIG. 2 a flow cytometric/FACS cytotoxicity assay showing the effect of aNK cell lysate that has been either sonicated or frozen-thawed on K562 cells.

FIG. 3 shows an immunoblot analysis identifying perforin and granzymes in lysate generated from aNK, haNK, and KHYG-1 cells.

FIG. 4A is a bar graph showing granzyme B activity of fresh and cryopreserved aNK, haNK, and KHYG-1 cell lysate.

FIG. 4B shows immunoblot analysis of perforin, granulysin, GzmA, GzmB, GzmH, GzmK, GzmM in lysates generated from NK-92 and haNK cells after repeated freeze/thaw cycles. The expression levels of the vinculin-1 protein were used as a protein loading control. Whole cell extracts using a conventional denaturing lysis buffer was used for comparison.

FIG. 4C shows immunoblot analysis of exosomal markers in lysates generated from NK-92 and haNK cells after repeated freeze/thaw cycles. The expression levels of the vinculin-1 protein were used as a protein loading control. Whole cell extracts using a conventional denaturing lysis buffer was used for comparison.

FIG. 5A and FIG. 5B are bar graphs showing the spectrum of cytokines and chemokines in aNK, haNK, and KHYG-1 cell lysate.

FIG. 5C shows MSD analysis of cytokine and chemokine concentrations in cell lysates from NK-92 and haNK cells (n=2). Only detectable concentrations in the MSD panel are presented here.

FIG. 6A and FIG. 6B are bar graphs indicating the concentration of IL-2 (FIG. 6A) and INF-γ (FIG. 6B) in aNK, haNK, and KHYG-1 cell lysate.

FIG. 6C and FIG. 6D are bar graphs indicating the concentration of IL-16 (FIG. 6C) and INF-γ (FIG. 6D) in aNK, haNK, and KHYG-1 cell lysates and extracts (supernatant).

FIG. 7 is a western blot identifying IL-16 precursor and active forms in cryopreserved aNK, haNK, and KHYG-1 cell lysate prepared by the freeze-thaw method compared to aNK, haNK, and KHYG-1 cell lysate prepared by detergent-based (RIPA SDS buffer) cell lysis.

FIG. 8A, FIG. 8B, FIG. 8C, FIG. 8D, and FIG. 8E are line graphs indicating the number of live CTAC (FIG. 8A), MX-1 (FIG. 8B), DH-82 (FIG. 8C), SKOV-3 (FIG. 8D), and A549 (FIG. 8E) cells after hours of incubation with varying concentrations of haNK cell lysate.

FIG. 9A-9G show cytotoxicity and anti-proliferative activity of haNK cell lysate on malignant cell lines. Percent target cell lysis by haNK lysate after 15-minute exposure is shown for human cell lines SKOV-3 (FIG. 9A) and SKBR-3 (FIG. 9B); canine cell lines OSCA-40 (FIG. 9C) and CTAC (FIG. 9D), as well as primary human non-malignant primary lung fibroblasts (FIG. 9E) and umbilical vein endothelial cells (HUVEC; FIG. 9F) (n=3). Cell doubling time of each cell line (FIG. 9G) was determined after exposure to cell medium, haNK lysate, or a balanced salt solution (BSS).

FIG. 9H-9J show cytotoxicity activity of haNK cell lysate on cell lines MDA-MB-231 (FIG. 9H), MC-38 (FIG. 9I), and DH-82 (FIG. 9J).

FIG. 10A and FIG. 10B show human target cell proliferation (FIG. 10A) and canine target cell proliferation (FIG. 10B) after 15-minute co-incubation with haNK lysate.

FIG. 11A and FIG. 11B show perforin activity of fresh aNK lysate on OSCA-40 cells. FIG. 11A shows number of live cells. FIG. 11B shows % target cell lysis.

FIG. 12A-12C show Granzyme B activity of fresh or previously frozen NK-92 and haNK cell lysates, and haNK cell lysate activity in vitro. FIG. 12A: Granzyme B activity (U/mL) is shown for both fresh and previously cryopreserved NK-92 and haNK cell lysates (n=2). Target cell lysis (% cytotoxicity) (FIG. 12B) and number of live cells (FIG. 12C) of both fresh and previously cryopreserved haNK cell lysate against canine OSCA-40 cells in the Incucyte (n=3). DMEM plus 10% FBS (“medium”) and Balanced Salt Solution (BSS) served as controls.

FIG. 13A-13D show the in vivo study design, tumor volume, survival and post-rechallenge of mice implanted with MC-38 tumors treated with haNK lysate. FIG. 13A: Shown is the study design. FIG. 13B: Tumor volume (mm³) over days post-enrollment measured up to Day 14; data graphed as the mean+SD. FIG. 13C: Overall survival of mice with primary tumor up to Day 49. FIG. 13D: Survival of rechallenged (n=4) and age-matched naïve control (n=5) mice, starting on Day 50 (arrow).

FIG. 14A shows tumor growth from day 0 to day 11 of the in vivo study.

FIG. 14B shows tumor volume on day 11 of the in vivo study.

FIG. 14C shows % survival of mice administered with different treatments.

FIG. 14D shows tumor volume of mice rechallenged with tumor cells.

### DETAILED DESCRIPTION OF THE INVENTION

### A. Introduction

In one aspect, a natural killer (NK) cell lysate including interleukin-16 (IL-16) is provided herein. An NK cell lysate may be prepared by detergent-free cell lysis, such as by one or more cycles of sonication or one or more cycles of freezing-thawing NK cells. The isolation procedure and analysis of cell lysate from NK cells or cell lines has not been reported. It was discovered that the lysate from NK cells contains molecules that are not expected to be present, such as IL-16. As IL-16 has not been described as an NK cell cytokine before, it was unexpected for IL-16 to be present in an NK cell lysate of the present disclosure. NK cell lysates lack several immune-active cytotoxic molecules in the lysate that are unexpectedly present in the NK cell lysate disclosed herein, hence it cannot be assumed that a lysate from an NK cell would have such specific and cytotoxic activity as an NK cell lysate described herein.

Some NK cell types require irradiation before the cells are infused into a patient. The nucleus contains genetic abnormalities that could program a malignant transferable phenotype in a recipient cell. Additionally, NK cells can express MHC class I and/or MHC class II antigens which can induce a cellular and humoral allogeneic immune response, potentially affecting their functionality/activity in a subject. Thus, the isolated and enriched NK cell lysate described herein is clinically superior to whole NK cells. The lysate is not known to contain replicating cells or genetic material that can transfer a malignant phenotype, and it also does not contain MHC molecules that could provoke an alloimmune response. Another clinical advantage of an NK cell lysate described herein is that it is enriched for immunomodulators, such as IL-16, which can stimulate B-cell function (e.g., antibody production) and can be a chemoattractant for CD4+ T-cells.

Without being bound by theory, upon local injection into a tumor site, an NK cell lysate is expected to break open the cell membrane of malignant cells and provide cytokines and other molecules that stimulate other immune cells (e.g., dendritic cells, monocytes, T-cells, etc.) in the tumor microenvironment. In addition to injection of an NK cell lysate into tumors, injection of an NK cell lysate into an infectious focus (i.e., abscess) can induce a similar cytotoxic reaction.

Thus, an NK cell lysate can be used to stimulate an immune response in a subject, for example to prevent and/or treat an infection and/or cancer, and/or initiate, enhance, or prolong an antitumor response.

Example aspects are provided so that this disclosure will be thorough and will fully convey the scope to those who are skilled in the art. Numerous specific details are set forth such as examples of specific compositions, components, and methods, to provide a thorough understanding of aspects of the present disclosure. It will be apparent to those skilled in the art that specific details need not be employed, that example aspects may be embodied in many different forms and that neither should be construed to limit the scope of the disclosure. In some example aspects, well-known processes, and well-known technologies are not described in detail.

### B. Definitions

The terminology used herein is for the purpose of describing particular example aspects only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various aspects set forth herein, in certain aspects, the term may alternatively be understood to instead be a more limiting and restrictive term, such as “consisting of” or “consisting essentially of” Thus, for any given aspect reciting compositions, materials, components, elements, features, integers, operations, and/or process steps, the present disclosure also specifically includes aspects consisting of, or consisting essentially of, such recited compositions, materials, components, elements, features, integers, operations, and/or process steps. In the case of “consisting of,” the alternative aspect excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an aspect, but any compositions, materials, components, elements, features, integers, operations, and/or process steps that do not materially affect the basic and novel characteristics can be included in the aspect.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one aspect,” “an aspect,” “some aspects,” “one example,” “for example,” or “an example” means that a particular element, feature, structure, or characteristic described in connection with the aspect is included in at least one aspect. The appearance of the phrase “in some aspects” or “one example” in various places in the specification is not necessarily all referring to the same aspect, for example. Further, all references to one or more aspects or examples are to be construed as non-limiting to the claims.

Throughout this disclosure, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art. Particularly in reference to a given quantity, number or percentage, “about” is meant to encompass deviations of plus or minus ten percent (±10). For example, about 5% encompasses any value between 4.5% to 5.5%, such as 4.5, 4.6, 4.7, 4.8, 4.9, 5, 4.1, 5.2, 5.3, 5.4, or 5.5. Accordingly, unless otherwise indicated, the numerical parameters set forth in this specification and attached claims are approximations that can vary depending upon the desired properties sought to be obtained by the presently disclosed subject matter.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB.

Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As will be understood by one skilled in the art, for any and all purpose, all ranges disclosed herein also encompass any and all possible subranges and combinations of subranges thereof. Furthermore, as will be understood by one skilled in the art, a range includes each individual member.

The term “agent” as used herein refers to a peptide, nucleic acid molecule, or small compound. An exemplary therapeutically active agent is IL-16.

The term “disease” as used herein refers to any condition or disorder that damages or interferes with the normal function of a cell, tissue, or organ. Examples of diseases include tumors and/or neoplasias and viral infections.

The term “reduce” as used herein refers to a negative alteration of at least 5%, 10%, 15%, 20%, 25%, 50%, 75%, or 100%.

The term “endogenous” as used herein refers to a substance e.g., a nucleic acid, protein, enzyme, etc. that is produced from within a cell (e.g., an NK cell) and/or that is naturally occurring or naturally found inside a cell. Thus, an endogenous nucleic acid sequence, gene, polynucleotide, or polypeptide refers to a nucleic acid sequence, gene, polynucleotide, or polypeptide produced by and found inside the cell. In some exemplary aspects an endogenous polypeptide or polynucleotide is encoded by the genome of the parental cell (or host cell). In other exemplary aspects, an endogenous polypeptide or polynucleotide is encoded by an autonomously replicating plasmid carried by the parental cell (or host cell). In some exemplary aspects, an endogenous gene or nucleic acid sequence is a gene or nucleic acid sequence that was present in the cell when the cell was originally isolated from nature, i.e., the gene is native to the cell. For example, an “endogenously produced” protein or enzyme may be expressed/generated by a cell's (e.g., an NK cell's) own machinery. In other words, the cell (e.g., NK cell) has not been genetically engineered to produce the protein or enzyme. In other words, a cell may endogenously produce native or non-native proteins/enzymes.

In contrast, an “exogenous” nucleic acid sequence, gene, polynucleotide, or polypeptide (e.g., an enzyme), or other substance (e.g., small molecule compound, etc.), as used herein, refers to a nucleic acid sequence, gene, polynucleotide, or polypeptide or other substance that is not encoded by or produced by the cell, and which is therefore added to a cell, a cell culture, or assay, from outside of the cell. For example, “exogenously added” may refer to adding a component, such as a cytokine as described herein, to the NK cell lysate described herein. A nucleic acid sequence encoding a variant (i.e., mutant) polypeptide, when added to the cell, is one example of an exogenous nucleic acid sequence. The exogenous nucleic acid sequence can encode a polypeptide or an enzyme that is also otherwise endogenous or native to the cell. Such an encoded polypeptide or enzyme can be considered “exogenously expressed.” For example, to achieve overexpression of an endogenous gene, additional copies of the gene can be introduced into the cell (e.g., in a vector, such as a plasmid). Example vector types include DNA vectors, RNA vectors, adenoviral vectors, and E2b-Ad5 vectors. Such additional copies of the endogenous gene can be considered as “exogenous” (e.g., exogenous gene(s) or an exogenous nucleic acid sequence(s)), because the additional copies are introduced into the cell from outside the cell. An “exogenous gene” or “exogenous nucleic acid sequence” also refers to a native (or endogenous) gene or nucleic acid sequence that is deregulated (e.g., upregulated or attenuated) or otherwise altered or modified, for example, by operably linking it to a regulatory element, such as a heterologous, or non-native, or non-naturally occurring, regulatory element (e.g., a promoter, enhancer, 5′-UTR, ribosome binding site, etc.); such a deregulated or altered gene or nucleic acid sequence can be on a chromosome or can be on a plasmid. An exogenous nucleic acid sequence or exogenous gene can also be used to express or overexpress a heterologous polypeptide or enzyme in a cell. Thus, an exogenous nucleic acid sequence or an exogenous gene can encode a polypeptide (e.g., an enzyme) that is native to the cell, that is otherwise endogenous to the cell, or that is heterologous to the cell.

The term “heterologous” as used herein refers to a polypeptide or polynucleotide which is in a non-native state. Thus, a polynucleotide or a polypeptide is “heterologous” to a cell when the polynucleotide and/or the polypeptide and the cell are not found in the same relationship to each other in nature. Therefore, a polynucleotide or polypeptide sequence is “heterologous” to an organism or a second sequence if it originates from a different organism, different cell type, or different species, or, if from the same species, it is modified from its original form. Thus, in an exemplary aspect, a polynucleotide or polypeptide is “heterologous” when it is not naturally present in a given organism.

Similarly, a polynucleotide or polypeptide is heterologous when it is modified from its native form or from its relationship with other polynucleotide sequences or is present in a recombinant host cell in a non-native state. Thus, in an exemplary aspect, a heterologous polynucleotide or polypeptide comprises two or more subsequences that are not found in the same relationship to each other in nature. For example, a promoter operably linked to a nucleotide coding sequence derived from a species different from that from which the promoter was derived. Alternatively, in another example, if a promoter is operably linked to a nucleotide coding sequence derived from a species that is the same as that from which the promoter was derived, then the operably-linked promoter and coding sequence are “heterologous” if the coding sequence is not naturally associated with the promoter (e.g. a constitutive promoter operably linked to a developmentally regulated coding sequence that is derived from the same species as the promoter). In other exemplary aspects, a heterologous polynucleotide or polypeptide is modified relative to the wild-type sequence naturally present in the corresponding wild-type host cell, e.g., an intentional modification e.g., an intentional mutation in the sequence of a polynucleotide or polypeptide or a modification in the level of expression of the polynucleotide or polypeptide. Typically, a heterologous nucleic acid or polynucleotide is recombinantly produced. A heterologous polynucleotide, polypeptide, or enzyme, for example, is typically exogenous to the cell, or exogenously expressed (or overexpressed) in the cell, i.e., is introduced into or added to the cell from outside the cell.

As used herein, “recombinantly produced” refers to production of a protein/enzyme by genetically engineering a cell to produce the protein/enzyme. The cell may be genetically engineered (referred to as genetically engineered or recombinant cell) to produce a protein/enzyme that is native to the cell (e.g., overproduction of a certain native protein/enzyme) or non-native to the cell meaning the gene that encodes the protein/enzyme is not found in the cell's natural genome.

As used herein the term “native” refers to the form of a nucleic acid, protein, polypeptide or a fragment thereof that is isolated from nature, or to a nucleic acid, protein, polypeptide or a fragment thereof that is in its natural state without intentionally introduced mutations in the structural sequence and/or without any engineered changes in expression such as e.g., changing a developmentally regulated gene to a constitutively expressed gene. As used herein, “native” also refers to “wildtype” or “wild-type,” in which the nucleic acid, protein, polypeptide, or a fragment thereof is present in both sequence, quantity, and relative quantity as typically found in the organism as naturally found. Wild-type organisms may serve as a control and/or reference for determination of cellular functions. A native gene, nucleic acid sequence, polypeptide, or enzyme, for example, is typically endogenous to a cell, i.e., found in or produced by the cell. An exogenous nucleic acid sequence or an exogenous gene can encode a native polypeptide or enzyme, for example, where additional copies of a native gene or nucleic acid sequence are added to the cell from outside the cell, or where a native gene or nucleic acid sequence is deregulated or altered, e.g., by operably coupling it to a regulatory element that is not native or endogenous to the cell.

The term “non-native” is used herein to refer to nucleic acid sequences, amino acid sequences, polypeptide sequences, enzymes, and/or small molecules that do not occur naturally in the host. Heterologous genes and polypeptides are considered “non-native.” A nucleic acid sequence or amino acid sequence that has been removed from a host cell, subjected to laboratory manipulation, and introduced or reintroduced into a host cell, is also considered “non-native.” Synthetic or partially synthetic genes introduced into a host cell are “non-native.” Non-native genes further include genes that are endogenous and/or native to the host microorganism but that are operably linked to one or more heterologous regulatory sequences that have been recombined into the host genome. A naturally occurring gene under the control of a heterologous regulatory sequence is considered “non-native.” In some aspects, an organism comprising a non-native gene may be utilized as a control and/or reference for an organism having additional and/or different variations from wildtype organisms.

Any compositions or methods provided herein can be combined with one or more of any of the other compositions and methods provided herein.

Unless defined otherwise, technical and scientific terms used herein have the same meaning as commonly understood by a person of ordinary skill in the art. In particular, this disclosure utilizes routine techniques in the field of immunology.

### C. NK Cell Lysate

In one aspect, a natural killer (NK) cell lysate is provided herein. In some aspects, an NK cell lysate may include one or more immunomodulators. In some aspects, an NK cell lysate may include a cytotoxic protein and a cytokine. In some aspects, an NK cell lysate can include a salt solution, a cytotoxic protein, and a cytokine. In some aspects, the cytokine is interleukin-16 (IL-16). In some aspects, the IL-16 is endogenously produced by NK cells.

A NK cell is a type of immune cell that has lytic granules (specialized organelles) with enzymes that can kill tumor cells and/or cells infected with a virus. It is a type of white blood cell and one skilled in immunology is aware of what an NK cell is. As used herein, NK cell, may refer to any natural killer cell. In certain aspects, the NK cell may be (i.e., may be derived from) an aNK cell (also known as an NK-92 cell), a haNK cell, a T-haNK cell, a primary NK cell, a KHYG-1 cell, or a variant or derivative thereof. In some aspects, the NK cell can be genetically engineered and in other aspects it cannot be genetically engineered. In some aspects, the NK cell does not express T or B cell receptors. In some aspects, the NK cell may be recognized by absence of CD3. The NK cell may additionally or alternatively be classified as CD56^{bright} or CD56^{dim}. In some aspects, the NK cell may be a memory-like cytokine enhanced (m-CENK) cell. In some aspects, the “NK cell” may be referring to a cell line.

NK-92 is an NK-like cell line that was initially isolated from the blood of a subject suffering from a large granular lymphoma and subsequently propagated in cell culture. The NK-92 cell line has been described (see e.g., Klingemann & al. (2016) Front Immunol 7:91). NK-92 cells have a CD3⁻/CD56⁺ phenotype that is characteristic of NK cells. They express all of the known NK cell-activating receptors except CD16, but lack most of the known NK cell inhibitory receptors. In some cases, the NK-92 cells express NKG2 A/CD94 and ILT2/LIR1 at low levels. In some cases, the NK-92 cells express at least one killer cell immunoglobulin-like receptor (KIR), for example, without limitation, KIR2DL4, which is a KIR with activating function and inhibitory potential, and is expressed by all NK cells. Furthermore, NK-92 is a clonal cell line. Unlike the polyclonal NK cells isolated from blood, NK-92 cells express these receptors in a consistent manner with respect to both type and cell surface concentration. While NK-92 cells can be (mildly) immunogenic and, in some cases, can (mildly) elicit an immune rejection response when administered therapeutically to a human subject, lysates of NK-92 cells are even less so. NK-92 cells and their lysates are well tolerated in humans with no known detrimental effects on normal tissues.

In some aspects, NK cells described herein may express a high affinity Fc-receptor. “High affinity” natural killer (haNK) cells are derived from the NK-92 cell line and have been engineered to express the high affinity CD16 allele (NK-92-CD16, also known as CD16/FcεRIγ-NK-92). Sequences for high-affinity variants of the Fcγ receptor are known (see e.g., Blood (2009) 113:3716-25). Expression of such receptor may increase specific targeting and cytotoxic cell killing of tumor cells when using antibodies that are specific to a patient's tumor cells. CD16 is commonly found in a form that has a relatively low binding affinity for the Fc portion of IgG molecules. An alternative form that exhibits a higher binding affinity is found in some individuals. The low and high affinity forms of CD16 differ only by the substitution of valine (high affinity) for phenylalanine (low affinity) at position 158 in the polypeptide chain. The complete sequences of the low and high affinity forms can be found in the SwissProt database as entries P08637 and VAR 008801, respectively. haNK cells expressing an antigen-binding, scFv are referred to herein as T-haNK cells. T-haNK cells capable of CD16, for example, in addition to a chimeric antigen receptor (CAR) having an intracellular domain of FcεRIγ, are discussed in detail in U.S. Pat. No. 11,643,452.

An NK cell lysate refers to a lysate generated by lysing at least one NK cell. An NK cell lysate may be prepared from at least one NK cell, where the lysate lacks NK cell membranes. Such an NK cell lysate is prepared from NK cells that have been lysed, i.e., NK cells in which the cell membranes have been disrupted, exposing NK cell contents to the rest of the composition. An NK cell may be lysed by known methods, such as by electrical, physical, chemical, and enzymatic techniques. Example disruption methods include high pressure or liquid homogenization, ultrasonication, sonication, freeze-thaw, and manual grinding, or detergent/solution-based cell lysis.

In some aspects, an NK cell lysate may refer to a lysate generated by lysing more than one NK cell type. For example, more than one NK cell type may be present in a solution and lysed substantially at the same time or about the same time or simultaneously generate an NK cell lysate. An NK cell lysate may be a combination of more than one NK cell lysate. For example, an NK cell lysate generated from one NK cell type may be combined with an NK cell lysate generated from a different NK cell type. In other words, the NK cell lysates may be combined after lysis. In some aspects, lysates generated from the same NK cell type may be combined after lysis.

In some aspects, an NK cell lysate is generated by one or more cycles of sonication. In a particular aspect, an NK cell lysate is prepared by at least three cycles of sonication. In some aspects, an NK cell lysate is prepared by freeze-thaw. In a particular aspect, an NK cell lysate is prepared by at least three freeze-thaw cycles. NK cells may be placed in liquid nitrogen for about 5, 10, 15, 20, 25, 30, 35, 40, 45, 50, 55, or 60 seconds during the “freeze” portions of the freeze-thaw cycles. NK cells may be placed in a water bath at about 35, 36, 37, 38, 39, or 40 °C for about 2 minutes during the “thaw” portions of the freeze-thaw cycles. In a particular aspect, one “freeze” portion consists of placing NK cells in liquid nitrogen for about 30 seconds, and one “thaw” portion consists of placing NK cells in an about 37 °C water bath until the NK cells are thawed. In some aspects, NK cells are kept in a water bath for about 10, 20, 30, 40, 50, 60, 70, 80, 90, 100, 110, 120, 130, 140, 150, 160, 170, or 180 seconds. In a certain aspect, NK cells are kept in a water bath for about 120 seconds.

In some aspects, an NK cell lysate may be further treated to substantially remove intact NK cells, NK cell debris, and/or NK cell genomic DNA, by any suitable method to produce an NK cell lysate substantially lacking intact NK cells, NK cell debris, and/or NK cell genomic DNA. As used herein, an NK cell lysate substantially lacking intact NK cells, NK cell debris, and/or NK cell genomic DNA refers to an NK cell lysate that has had at least 60%, at least 60%, at least 70%, at least 80%, at least 90%, at least 95%, at least 98%, at least 99%, or almost all intact NK cells, NK cell debris, and/or NK cell genomic DNA removed from the NK cell lysate. In some aspects, NK cell debris can include MHC class I or II molecules, cell membranes, and/or organelles. In some aspects, the NK cell lysate is substantially lacking MHC class I and/or MHC class II molecules. For example, NK cell membranes can be removed from lysed NK cells by centrifugation to produce a lysate (supernatant). In some aspects, NK cells may be centrifuged for about 1, 2, 3, 4, 5, 6, 7, 8, 9, 10, 11, 12, 13, 14, or 15 minutes, In some aspects, NK cells may be centrifuged at about 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, or 25,000 g. In some aspects, NK cells may be centrifuged at about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 °C. In a specific aspect, NK cells may be centrifuged for about 10 minutes, at about 3,000 g, at about 4 °C. Alternatively, an NK cell lysate can be cleared of cell membrane debris after rupture by means other than centrifugation. For example, an alternate method may include filtration or treatment of cells with conA beads.

In some aspects, the NK cells can be expanded prior to lysis. Optionally, NK cells can be expanded to a density of about 1×10⁶ cells/mL prior to lysis. Optionally, NK cells can be expanded to a density of at least 1×10⁶ cells/mL prior to lysis. In some aspects, NK cells can be expanded with cytokines, such as IL-2, IL-12, IL-15, IL-18, or a combination thereof. In one aspect, the NK cells can be expanded with IL-2. The NK cells may be expanded, for example with IL-2, in a cell culture medium. In some aspects, the interleukin (IL) may be an endoplasmic reticulum targeted IL, such as IL-2 (erIL-2), IL-12 (erIL-12), IL-15 (erIL-15), or IL-18 (erIL-18).

In some aspects, the polypeptide sequence for targeting a protein to the endoplasmic reticulum (ER) is Gly Ser Glu Lys Asp Glu Leu (SEQ ID NO: 1). In some aspects, the targeting sequence (SEQ ID NO: 1) is covalently linked to IL-2 to produce the erIL-2 polypeptide. In some aspects, the targeting sequence (SEQ ID NO: 1) is covalently linked to IL-12 to produce the erIL-12 polypeptide. In some aspects, the targeting sequence (SEQ ID NO: 1) is covalently linked to IL-15 to produce the erIL-15 polypeptide. In some aspects, the targeting sequence (SEQ ID NO: 1) is covalently linked to IL-18 to produce the erIL-18 polypeptide.

In some aspects, the polypeptide sequence for wild-type IL-2 is as shown below:
Met Tyr Arg Met Gln Leu Leu Ser Cys Ile Ala Leu Ser Leu Ala Leu Val Thr Asn Ser Ala Pro Thr Ser Ser Ser Thr Lys Lys Thr Gln Leu Gln Leu Glu His Leu Leu Leu Asp Leu Gln Met Ile Leu Asn Gly Ile Asn Asn Tyr Lys Asn Pro Lys Leu Thr Arg Met Leu Thr Phe Lys Phe Tyr Met Pro Lys Lys Ala Thr Glu Leu Lys His Leu Gln Cys Leu Glu Glu Glu Leu Lys Pro Leu Glu Glu Val Leu Asn Leu Ala Gln Ser Lys Asn Phe His Leu Arg Pro Arg Asp Leu Ile Ser Asn Ile Asn Val Ile Val Leu Glu Leu Lys Gly Ser Glu Thr Thr Phe Met Cys Glu Tyr Ala Asp Glu Thr Ala Thr Ile Val Glu Phe Leu Asn Arg Trp Ile Thr Phe Cys Gln Ser Ile Ile Ser Thr Leu Thr (SEQ ID NO: 2)

In some aspects, the nucleic acid (coding) sequence for erIL-2 is as shown below:
atgtaccggatgcagctgctgagctgtatcgccctgtctctggccctcgtgaccaacagcgcccctaccagcagcagcaccaagaaaacc cagctgcagctggaacatctgctgctggacctgcagatgatcctgaacggcatcaacaactacaagaaccccaagctgacccggatgctg accttcaagttctacatgcccaagaaggccaccgaactgaaacatctgcagtgcctagaagaggaactgaagcccctggaagaagtgctg aacctggcccagagcaagaacttccacctgaggcccagggacctgatcagcaacatcaacgtgatcgtgctggaactgaaaggcagcga gacaaccttcatgtgcgagtacgccgacgagacagctaccatcgtggaatttctgaaccgatggatcaccttctgccagagcatcatcagca ccctgaccggctccgagaaggacgagctgtga (SEQ ID NO: 3)

In some aspects, the polypeptide sequence for erIL-2 is as shown below:
Met Tyr Arg Met Gln Leu Leu Ser Cys Ile Ala Leu Ser Leu Ala Leu Val Thr Asn Ser Ala Pro Thr Ser Ser Ser Thr Lys Lys Thr Gln Leu Gln Leu Glu His Leu Leu Leu Asp Leu Gln Met Ile Leu Asn Gly Ile Asn Asn Tyr Lys Asn Pro Lys Leu Thr Arg Met Leu Thr Phe Lys Phe Tyr Met Pro Lys Lys Ala Thr Glu Leu Lys His Leu Gln Cys Leu Glu Glu Glu Leu Lys Pro Leu Glu Glu Val Leu Asn Leu Ala Gln Ser Lys Asn Phe His Leu Arg Pro Arg Asp Leu Ile Ser Asn Ile Asn Val Ile Val Leu Glu Leu Lys Gly Ser Glu Thr Thr Phe Met Cys Glu Tyr Ala Asp Glu Thr Ala Thr Ile Val Glu Phe Leu Asn Arg Trp Ile Thr Phe Cys Gln Ser Ile Ile Ser Thr Leu Thr Gly Ser Glu Lys Asp Glu Leu (SEQ ID NO: 4)

In some aspects, the transgene construct sequence for expressing erIL-2, e.g., as used in the haNK cells, is as shown below:
MYRMQLLSCI ALSLALVTNS APTSSSTKKT QLQLEHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLTGSEKDEL (SEQ ID NO: 5)

In some aspects, the expanded NK cells may be m-ceNK cells. In some aspects, the NK cells may be expanded to a density of at least 1×10¹, at least 1×10², at least 1×10³, at least 1×10⁴, at least 1×10⁵, at least 1×10⁶, at least 1×10⁷, at least 1×10⁸, at least 1×10⁹, at least 1×10¹⁰, at least 1×10¹¹, at least 1×10¹², at least 1×10¹³, at least 1×10¹⁴, or at least 1×10¹⁵ m-ceNK cells/mL. In a particular aspect, the NK cells may be expanded to a density of at least 1×10⁶ m-ceNK cells/mL. M-ceNK cells may be differentiated into specialized NK cells with enhanced anti-cancer function by brief pre-activation with IL-12, IL-15, and IL-18. M-ceNK cells may be characterized by their unique cell-surface marker profile and enhanced responses to cytokine re-stimulation that may include increased IFN-γ production and cytotoxicity against leukemic cell lines. M-ceNK cells may be generated from a mixture of mononuclear cells obtained from whole blood or cord blood. In some aspects, the efficacy of m-ceNK cells can be further amplified by the IL-15 superagonist nogapendekin-alfa-inbakicep (NAI which is also referred to herein as “N-803”). A “salt solution” as used to herein is a solution comprising salt and water. The salt may include sodium, potassium, calcium, magnesium, chloride, and/or other salts. In some aspects, the salt solution may include a tonicity adjusting agent. In some aspects, the salt solution is either hypertonic, hypotonic, or isotonic. In some aspects, glucose is added to the salt solution as an energy source. In some aspects, phenol red is used in the salt solution as a pH indicator. In some aspects, a salt solution may be a “balanced salt solution (BSS)”. A BSS, as used herein, refers to a solution made to a physiological pH and isotonic salt concentration. A BSS commonly includes sodium, potassium, calcium, magnesium, chloride, or a combination thereof. In some aspects, the salt solution may be made to a physiological pH and/or isotonic salt concentration. In some aspects, the salt solution may be used for washing NK cells, transporting NK cells, and/or diluting NK cells. In a specific aspect, the salt solution is used for washing NK cells. In some aspects, the salt solution may provide NK cells with water and/or inorganic ions. Additionally or alternatively, the salt solution may maintain an osmotic pressure. Non-limiting example salt solutions include: Alsever's solution, Earle's balanced salt solution (EBSS), Gey's balanced salt solution (GBSS), Hank's balanced salt solution (HBSS), (Dulbecco's) phosphate buffer saline (PBS), TRIS-buffered saline (TBS), etc. In a certain aspect, the salt solution is PBS. PBS, for example, is a water-based salt solution containing disodium hydrogen phosphate, sodium chloride, and in some formulations, potassium chloride and potassium dihydrogen phosphate. The buffer helps to maintain a constant pH, and the osmolarity and ion concentrations of the solutions match those of the human body (isotonic).

Optionally, the NK cells may be suspended in a salt solution prior to lysis. For example, the NK cells may be suspended in a salt solution prior to three rounds of freeze-thaw. In some aspects, additional components may be added to the salt solution to aid in NK cell lysis. Optionally, the NK cell lysate can contain a salt solution and at least one immunomodulator as described herein. In some aspects, the immunomodulator is cytokine IL-16. In some aspects, the IL-16 is endogenously produced by NK cells. In some aspects, an NK cell lysate can include an additional immunomodulator. In some aspects, the additional immunomodulator is a cytotoxic protein as described herein. In a specific aspect, an NK cell lysate can include a salt solution, a cytotoxic protein, and IL-16.

In some aspects, the salt solution and/or NK cell lysate may contain a detergent. Alternatively, the salt solution and/or NK cell lysate may not contain a detergent, such as sodium dodecyl sulphate (SDS), Triton™ X-100, Triton™ X-114, NP-40, Tween® 20, Tween® 80, CHAPS, CHAPSO, Brij-35, Brij-58, Octyl-beta-Glucoside, Octylthio Glucoside, etc. Detergents may act by disrupting the lipid bilayer of a cell membrane and may lead to permeabilization and release of intracellular components. They are often used in cell lysis procedures.

In some aspects, the salt solution and/or NK cell lysate may include ethylenediaminetetraacetic acid (EDTA). Alternatively, the salt solution/NK cell lysate may not include EDTA.

In some aspects, the NK cell lysate can be used in stimulating an immune response. Additionally or alternatively, the NK cell lysate can be used in preventing and/or treating an infection. Additionally or alternatively, the NK cell lysate can be used in preventing and/or treating cancer. Additionally or alternatively, the NK cell lysate can be used in initiating, enhancing, or prolonging an anti-tumor response. In some aspects, the NK lysate may contain tumoricidal and/or antimicrobial components as described in U.S. Pat. No. 10,258,649, which is hereby incorporated by reference in its entirety. In some aspects, the tumoricidal and/or antimicrobial components may be used to stimulate an immune response, prevent and/or treat an infection, prevent and/or treat cancer, and/or initiate, enhance, or prolong an anti-tumor response.

In some aspects, the NK cell lysate can be used fresh or after cryopreservation. The lysate may be cryopreserved in −70, −71, −72. −73, −74, −75, −76, −77, −78, −79, −80, −81, −82, −83, −84, −85, −86, −87, −88, −89, or −90 °C for up to and including 1, 2, 3, 4, 5, 10, 15, 20, 25, 30, 35, 40, 45, 50, 60, 70, 80, 90, 100, or more years.

An “immunomodulator” as used herein is a type of immunotherapeutic agent. An immunomodulator may refer to any substance that alters (e.g., induces, enhances, restores, suppresses, etc.) an immune response, or to an agent that utilizes or is derived from a component of the immune system. In some aspects, immunomodulators may be immunotherapies, corticosteroids, traditional disease-modifying antirheumatic drugs (DMARDs), biologics, and Janus kinase inhibitors.

An immunomodulator may be endogenously and/or recombinantly produced by an NK cell. In certain aspects, the immunomodulator may be endogenously and/or recombinantly produced by NK cells prior to NK cell lysis. In other aspects, an immunomodulator may be exogenously added to the NK cells before lysis or to the NK cell lysate after lysing the NK cells. For example, in some aspects, an NK cell may be transformed with an expression vector (e.g., plasmid) that includes a promoter and an immunomodulator nucleotide sequence. In some example aspects, the expression vector may include one or more tumor antigens or neoantigens. In some aspects, the NK cell may be transformed with one or more expression vectors.

In some aspects, an immunomodulator can be a cytokine, agonist derivative of the cytokine, or fusion construct comprising the cytokine and/or agonist derivative of the cytokine. In some aspects, some cytokines may be considered chemokines. A cytokine may refer to substances, such as chemokines, interferons (INs), interleukins (ILs), lymphokines, tumor necrosis factors (TNFs), hormones, and growth factors, which may be secreted by immune system cells, and may have an effect on other cells. Cytokines may be produced by immune cells such as macrophages, B lymphocytes, T lymphocytes, and mast cells, as well as endothelial cells, fibroblasts, and various stromal cells. A given cytokine may be generated by more than one type of cell. Examples of cytokines include IL-1α, IL-1β, IL-2, IL-4, IL-5, IL-6, IL-7, IL-10, IL-11, IL-12p40, IL-12p70, IL-13, IL-15, IL-16, IL-17, IL-18, INF-γ, GM-CSF, G-CSF, TNF-α, TNF-β, VEGF, IL-8, Eotaxin-3, MCP-1, MCP-4, MDC, MIP-1α, MIP-1β, and TARC. In some aspects, the IL-15 may be an IL-15:IL-15Rα or a stabilized derivative thereof, such as nogapendekin-alfa-inbakicept (NAI which is also referred to herein as “N-803”). In some aspects, an NK cell lysate may include more than one cytokine. In some aspects, an immunomodulator may be an IL-15:IL-15Rα complex or fusion construct with IL-15 agonist activity derived therefrom. In some aspects, the IL-15:IL-15Rα complex, stabilized derivative thereof, or fusion construct is present in the NK cell lysate at 50-500 ng/mL, 100-500 ng/mL, 150-500 ng/mL, 50-400 ng/mL, 50-300 ng/mL, 50-200 ng/mL, about 100 ng/mL, about 150 ng/mL, or about 200 ng/mL.

In some aspects, the NK cell lysate may include one or more additional cytokines, agonist derivatives of the cytokines, fusion constructs comprising the cytokines and/or agonist derivatives of the cytokines, or a combination thereof. In some aspects, a fusion construct may include one or more cytokines and one or more agonist derivatives thereof. In some aspects, an agonist derivative of a cytokine may be a fusion protein with cytokine agonist activity.

An agonist derivative of a cytokine, or cytokine agonist, may refer to a compound or molecule that binds to and activates a receptor of said cytokine. For example, an “IL-15 agonist” may refer to a compound or molecule that binds to and activates the IL-15 receptor (“IL-15Rβγ”). The type of compound or molecule of the cytokine agonist is not particularly limited so long as it binds to and activates a receptor of said cytokine. For example, the type of compound or molecule of the IL-15 agonist is not particularly limited so long as it binds to and activates the IL-15Rβγ, particularly in complex with IL-15Rα. A cytokine agonist may be a peptide, protein, small molecule (e.g., a pharmaceutical drug), or oligonucleotide. The peptide or proteins may be a single amino acid sequence or two or more sequences bound via covalent attachments (e.g., disulfide bonds) or non-covalent attachments (e.g., hydrophilic or hydrophobic interactions, hydrogen bonds). In a particular aspect, a cytokine agonist is an antibody, modified antibody, chimeric antibody, or a derivative thereof. In some aspects, the cytokine agonist may be an IL-15 agonist. In further aspects, the IL-15 agonist may be a superagonist complex, such as an IL-15 derivative bound to an IL-15Rα or an IL-15Rα/IgG1 Fc fusion protein, also known as NAI. An exemplary IL-15 agonist may be selected from the list comprising IL-15, a fusion protein comprising IL-15, IL-15:IL-15Rα, and a fusion protein comprising IL-15:IL-15Rα.

In some aspects, the cytokine is IL-16. In some aspects, IL-16 is present in the NK cell lysate at 10,000-300,000 pg/mL, 20,000-300,000 pg/mL, 50,000-300,000 pg/mL, 70,000-300,000 pg/mL, 90,000-300,000 pg/mL, 120,000-300,000 pg/mL, 10,000-170,000 pg/mL, 20,000-170,000 pg/mL, 50,000-170,000 pg/mL, 70,000-170,000 pg/mL, 90,000-170,000 pg/mL, 120,000-170,000 pg/mL, 10,000-100,000 pg/mL, 20,000-100,000 pg/mL, 50,000-100,000 pg/mL, 70,000-100,000 pg/mL, or 90,000-100,000 pg/mL. In some aspects, the concentration of IL-16 in the lysate is normalized to 10e⁶ cells per mL of lysate. In some aspects, the concentration of IL-16 in the lysate is normalized to 1e⁸ cells per mL of lysate. In some aspects, the concentration of IL-16 in the lysate is normalized to 1e⁶ cells per mL of lysate.

In some aspects, the cytokine is IL-2. In some aspects, IL-2 is present in the NK cell lysate at 1,000-200,000 pg/mL, 2,000-200,000 pg/mL, 10,000-200,000 pg/mL, 50,000-200,000 pg/mL, 60,000-200,000 pg/mL, 70,000-200,000 pg/mL, 100,000-200,000 pg/mL, 120,000-200,000 pg/mL, 150,000-200,000 pg/mL, 1,000-100,000 pg/mL, 2,000-100,000 pg/mL, 10,000-100,000 pg/mL, 50,000-100,000 pg/mL, 60,000-100,000 pg/mL, or 70,000-100,000 pg/mL. In some aspects, the lysate is normalized to 10e⁶ cells per mL of lysate. In some aspects, the concentration of IL-16 in the lysate is normalized to 1e⁸ cells per mL of lysate. In some aspects, the concentration of IL-16 in the lysate is normalized to 1e⁶ cells per mL of lysate.

In some aspects, the cytokine is IL-10. In some aspects, IL-10 is present in the NK cell lysate at 50-10,000 pg/mL, 100-10,000 pg/mL, 2,000-10,000 pg/mL, 4,000-10,000 pg/mL, 5,000-10,000 pg/mL, or 6,000-10,000 pg/mL.

In some aspects, the cytokine is IL-13. In some aspects, IL-10 is present in the NK cell lysate at 50-2,000 pg/mL, 100-2,000 pg/mL, 500-2,000 pg/mL, or 1,000-2,000 pg/mL.

In some aspects, the cytokine is IL-17. In some aspects, IL-17 is present in the NK cell lysate at 50-2,000 pg/mL, 100-2,000 pg/mL, 500-2,000 pg/mL, or 1,000-2,000 pg/mL.

In some aspects, the cytokine is IFN-γ. In some aspects, IFN-γ is present in the NK cell lysate at 1,000-150,000 pg/mL 2,000-150,000 pg/mL, 5,000-150,000 pg/mL, 6,000-150,000 pg/mL, 8,000-150,000 pg/mL, 10,000-150,000 pg/mL, 1,000-100,000 pg/mL, 1,000-150,000 pg/mL, 50,000-150,000 pg/mL, 10,000-100,000 pg/mL, or 50,000-100,000 pg/mL.

In some aspects, the cytokine is TNF-β. In some aspects, TNF-β is present in the NK cell lysate at 2,000-100,000 pg/mL, 5,000-100,000 pg/mL, 6,000-100,000 pg/mL, 8,000-100,000 pg/mL, 10,000-100,000 pg/mL, 10,000-80,000 pg/mL, 10,000-60,000 pg/mL, or 10,000-50,000 pg/mL.

In some aspects, the cytokine is IL-8. In some aspects, IL-8 is present in the NK cell lysate at 1,000-10,000 pg/mL, 2,000-10,000 pg/mL, 3,000-10,000 pg/mL, 4,000-10,000 pg/mL, 5,000-10,000 pg/mL, 2,000-5,000 pg/mL, or 3,000-5,000 pg/mL.

In some aspects, the cytokine is MIP-1α. In some aspects, MIP-1a is present in the NK cell lysate at 2,000-100,000 pg/mL, 5,000-100,000 pg/mL, 6,000-100,000 pg/mL, 8,000-100,000 pg/mL, 10,000-100,000 pg/mL, 10,000-80,000 pg/mL, 10,000-60,000 pg/mL, or 10,000-50,000 pg/mL.

In some aspects, the cytokine is MIP-1β. In some aspects, MIP-1β is present in the NK cell lysate at 100-100,000 pg/mL, 200-100,000 pg/mL, 2,000-100,000 pg/mL, 5,000-100,000 pg/mL, 6,000-100,000 pg/mL, 8,000-100,000 pg/mL, 10,000-100,000 pg/mL, 5,000-80,000 pg/mL, 5,000-60,000 pg/mL, 5,000-50,000 pg/mL, 5,000-40,000 pg/mL, 5,000-30,000 pg/mL, 5,000-20,000 pg/mL, or 5,000-10,000 pg/mL. In some aspects, the lysate is normalized to 10e⁶ cells per mL of lysate. In some aspects, the concentration of IL-16 in the lysate is normalized to 1e⁸ cells per mL of lysate. In some aspects, the concentration of IL-16 in the lysate is normalized to 1e⁶ cells per mL of lysate.

In some aspects, the immunomodulator may be a cytotoxic molecule or protein. A cytotoxic molecule or protein as described herein refers to a molecule or protein that initiates and/or executes a cytotoxic effect. In some aspects, the cytotoxic protein may be referred to as a cytotoxic protease or a cytotoxic granule. Examples of cytotoxic proteins include gasdermin D (GSDMD), perforin 1 (PRF1), granulysin (GNLY), serine proteases, such as granzymes (e.g., human GzmA, GzmB, GzmH, GzmK and GzmM), and cathepsins. Cytotoxic proteins may form pores in cell membranes that can promote cytolysis and the release of intracellular proteins. In some aspects, cytotoxic proteins may be involved in cytotoxic T lymphocyte (CTL) elimination of target cells. In some aspects, cytotoxic proteins may be expressed by immune cells such as NK cells, mast cells, T cells, dendritic cells, etc. In some aspects, an NK cell lysate may include more than one cytotoxic molecule.

In some aspects, the immunomodulator may be a yeast lysate. In some aspects, a yeast lysate may be a yeast extract. A yeast lysate may be prepared from a yeast that lacks yeast membranes and yeast cell walls. A yeast lysate may contain one or more antigens that are endogenously produced or recombinantly produced or exogenously added to the yeast. In some aspects, the antigen may be endogenously expressed by the yeast. Additionally or alternatively, the antigen may be recombinantly produced by the yeast. The antigen can thus be native or non-native. In other aspects, the antigen may be exogenously added to the yeast lysate. In some aspects, the yeast lysate is generated from Saccharomyces cerevisiae.

Antigens, as referred to herein, may include any antigen against which it is desired to elicit an immune response, and, may include any antigen for which a therapeutic immune response against such antigen would be beneficial to an individual. For example, antigens can include, but are not limited to, antigens associated with a pathogen, including viral antigens, fungal antigens, bacterial antigens, helminth antigens, parasitic antigens, ectoparasite antigens, protozoan antigens, or antigens from any other infectious agent. Antigens may be associated with a particular disease or condition, whether from a pathogenic or cellular source, associated with an autoimmune disease (e.g., diabetes antigens), allergy antigens (allergens), mammalian cell molecules harboring one or more mutated amino acids, proteins normally expressed pre- or neo-natally by mammalian cells, proteins whole expression is induced by insertion of an epidemiologic agent (e.g., virus), proteins whose expression is induced by gene translocation, and proteins whose expression is induced by mutation of regulatory sequences. Antigens can be native antigens or genetically engineered antigens which have been modified in some manner (e.g., sequence change or generation of a fusion protein). In some aspects, an antigen can be a protein or any epitope or immunogenic domain thereof, a fusion protein, or a chimeric protein, rather than an entire cell or microorganism.

In some aspects, an NK cell lysate may include one or more additional immunomodulators besides IL-16. For example, the additional immunomodulator may be another cytokine, chemokine, yeast lysate, or a combination thereof. In a particular aspect, the additional immunomodulator is a chemokine, such as INF-γ.

IL-16 is a pro-inflammatory pleiotropic cytokine that can function as a chemoattractant, modulator of T cell activation, and/or inhibitor of HIV replication. IL-16 may act as an immunomodulatory cytokine that contributes to the regulatory process of CD4+ cell recruitment and activation at sites of inflammation. IL-16 may additionally or alternatively promote the amplification of CD4+CD25+ Tregs and the enrichment of Foxp3+ cells. IL-16 may play a role in initiating and/or sustaining the inflammatory response. IL-16 may also promote the secretion of other pro-inflammatory cytokines, such as tumor necrosis factor-α (TNF-α), IL-1β, IL-6, and IL-15, which may be involved in tumorigenesis/carcinogenesis/oncogenesis (i.e., the process of normal cells gaining malignant properties). Thus, IL-16 may additionally or alternatively play a role in tumorigenesis. IL-16 may also be referred to as lymphocyte chemoattractant factor (LCF). In some aspects, the signaling process of IL-16 may be mediated by CD4.

The IL16 gene product, pro-interleukin-16 (pro-IL-16), encoded by the IL16 gene, can undergo proteolytic processing to yield at least one functional protein/active form. Alternative splicing may result in more than one transcript variant. In some aspects, proteolytic processing may result in two functional proteins/active forms. The proteolytic processing may involve enzymatically cleaving pro-IL-16 with a protease, such as caspase-3, which may release one or more active forms of IL-16. IL-16's cytokine function may be from the secreted C-terminal peptide, and the N-terminal product may play a role in cell cycle control. In some aspects, pro-IL-16 may be made up of 631 amino acids. In some aspects, caspace-3 may cleave pro-IL-16 to release a bioactive IL-16 cytokine. In some aspects, an IL-16 cytokine may be secreted.

Additionally, the sequence and overall structure of IL-16 is conserved across species, and can be found in both humans and mice, for example. The human pro-IL-16 may display over 90% sequence similarity to various non-human primates. As used herein, “IL-16” may refer to any active form of IL-16 generated by proteolytic processing of pro-IL-16.

In some aspects, IL-16 may be used to prevent, reduce the occurrence of, and/or treat a disease. For example, IL-16 may be used to treat infectious, immune-mediated, and/or autoimmune inflammatory disorders, such as atopic dermatitis, irritable bowel syndrome, systemic lupus erythematosus, neurodegenerative disorders, and viral infections.

In some aspects, IL-16 may be generated by the NK cell. Thus, the NK cell lysate can include IL-16. In some aspects, the NK cell lysate can include at least about 10, 20, 30, 40, 50, 100, 200, 500, 1,500, 2,000, 2,500, 3,000, 3,500, or 4,000 pg/mL of IL-16. In a particular aspect, the NK cell lysate can include at least about 2,000 pg/mL of IL-16. In some aspects, the NK cell lysate may comprise at least about 20,000, 40,000, 60,000, 80,000, or 100,000 pg/mL of IL-16.

In some aspects, the NK cell lysate can include an immunomodulator in addition to the cytokine IL-16. In some aspects, the additional immunomodulator is INF-γ.

In some aspects, the NK cell lysate can be produced by one or more cycles/rounds of sonication or one or more cycles/rounds of freezing-thawing. In some aspects, the NK cell lysate is generated by 1, 2, 3, 4, 5, 6, 7, 8, 9, or 10 cycles of sonication or freezing-thawing. In one aspect, the NK cell lysate is generated by three cycles of freezing-thawing. Methods of preparing the NK cell lysate are described below.

### D. Methods of Preparing the NK Cell Lysate

In a further aspect, the NK cell lysate can be prepared by lysing one or more NK cells. In some aspects, the NK cell lysate is derived from NK-92/aNK cells, haNK cells, t-haNK cells, primary NK cells, KHYG-1 cells, or variants thereof. Intact NK cells, NK cell debris, and NK cell genomic DNA may be removed from the lysate. In some aspects, NK cell debris may include MHC class I and/or II molecules, cell membranes, and/or organelles.

Prior to lysis, the NK cells may be suspended in a salt solution as described above. Additionally, prior to lysis, the NK cells may be expanded. As mentioned above, the NK cells may be expanded with IL-2, IL-12, IL-15, IL-18, or a combination thereof. In some aspects, the NK cells may be expanded to a density of about 1×10¹, about 1×10², about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, about 1×10¹⁴, or about 1×10¹⁵ cells/mL. In a particular aspect, the NK cells may be expanded to a density of about 1×10⁶ cells/mL. In some aspects, the NK cells may be expanded to a density of at least 1×10¹, at least 1×10², at least 1×10³, at least 1×10⁴, at least 1×10⁵, at least 1×10⁶, at least 1×10⁷, at least 1×10⁸, at least 1×10⁹, at least 1×10¹⁰, at least 1×10¹¹, at least 1×10¹², at least 1×10¹³, at least 1×10¹⁴, or at least 1×10¹⁵ cells/mL. In a particular aspect, the NK cells may be expanded to a density of at least 1×10⁶ cells/mL.

In some aspects, the NK cell lysate can be prepared by disrupting the NK cell membranes. For example, the NK cell lysate can be generated by electrical, physical, chemical, and enzymatic techniques. Example disruption methods include high pressure or liquid homogenization, ultrasonication, sonication, freeze-thaw, and manual grinding, or detergent/solution-based cell lysis. In one aspect, the NK cell lysate is prepared by the freeze-thaw method. In some aspects, the NK cell lysate may be prepared by 1, 2, 3, 4, 5, 6, 7, 8, 9, or 10 cycles of freezing-thawing. In a particular aspect, an NK cell lysate is prepared by at least three cycles of freezing-thawing. In some aspects, no detergent is added to the salt solution. Thus, in these aspects, there is no detergent present to aid NK cell lysis.

After lysis, the NK cell lysate may be further treated to remove intact NK cells, NK cell debris, and NK cell genomic DNA by any suitable method to produce an NK cell lysate lacking intact NK cells, NK cell debris, and NK cell genomic DNA. For example, NK cell membranes can be removed from lysed NK cells by centrifugation to produce a lysate (supernatant). The supernatant may be free from cell membranes. For example, NK cells may undergo centrifugation for about 10 minutes, at about 3,000 g, at about 4 °C. Alternatively, NK lysates can be cleared of cell membrane debris after rupture by means other than centrifugation, for example by filtration or treatment of cells with conA beads.

In some aspects, the NK cell lysate prepared by the method described herein may be used to prevent, reduce the occurrence of, and/or treat cancer; prevent, reduce the occurrence of, and/or treat an infection; and/or initiate, enhance, or prolong an anti-tumor response.

### E. Methods of Producing IL-16 from NK Cells

A method for producing the cytokine, IL-16, from NK cells is described herein. The method may include lysing NK cells to produce a lysate, for example by sonication or freeze-thaw methods as described herein, and removing intact NK cells, NK cell debris, and NK cell genomic DNA from the lysate, for example by centrifugation. In some aspects, the NK cells may not be ruptured/lysed by a detergent (i.e., there is no detergent added during NK cell lysis). Additionally, as mentioned above, the NK cell lysate may be centrifuged to remove intact NK cells, NK cell debris, and/or NK cell genomic DNA. In some aspects, NK cell debris includes MHC class I and/or II, cell membranes, and/or organelles.

As mentioned, NK cells may be suspended in a salt solution prior to lysis. NK cells may additionally or alternatively be expanded in a cell culture medium prior to lysis. NK cells may be expanded with IL-2, IL-12, IL-15, IL-18, or a combination thereof, and may be expanded to a density of about 1×10¹, about 1×10², about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, about 1×10¹⁴, or about 1×10¹⁵ cells/mL. In a particular aspect, NK cells may be expended to a density of about 1×10⁶ cells/mL. In some aspects, the NK cells may be expanded to a density of at least 1×10¹, at least 1×10², at least 1×10³, at least 1×10⁴, at least 1×10⁵, at least 1×10⁶, at least 1×10⁷, at least 1×10⁸, at least 1×10⁹, at least 1×10¹⁰, at least 1×10¹¹, at least 1×10¹², at least 1×10¹³, at least 1×10¹⁴, or at least 1×10¹⁵ cells/mL. In a particular aspect, the NK cells may be expanded to a density of at least 1×10⁶ cells/mL.

As mentioned herein, NK cells may be (i.e., may be derived from) NK-92/aNK cells, haNK cells, t-haNK cells, primary NK cells, KHYG-1 cells, or a variant thereof. In some aspects, IL-16 is endogenously produced by the NK cells (i.e., by the cell's own machinery) and/or recombinantly produced by the NK cells (e.g., by genetic engineering). In some aspects, IL-16 is exogenously added to the NK cells, NK cell lysate, or composition comprising the NK cell lysate. In a specific aspect, IL-16 is endogenously produced by the NK cells. In some aspects, IL-16 is endogenously and/or recombinantly produced by NK cells prior to NK cell lysis. In certain aspects, the NK cells are not genetically engineered to express IL-16. Further, in some aspects, the NK cells may endogenously produce native or non-native IL-16. In additional aspects, the NK cells may endogenously produce IL-16 but also be genetically engineered (aka recombinant) to produce another immunomodulator or cytokine. For example, the NK cells can be genetically engineered to produce IL-2 or another cytokine while endogenously producing IL-16.

In additional aspects, IL-16 can be isolated from the NK cell lysate. IL-16, as well as other immunomodulators, may be isolated from the NK cells or NK cell lysate/composition by known protein purification methods. For example, immunomodulators, such as cytokines, may be isolated from the NK cells or NK cell lysate/composition by extraction, precipitation, centrifugation, dialysis, chromatography, electrophoresis, affinity chromatography, mass spectrometry, etc. In a certain aspect, IL-16 may be isolated by affinity chromatography, such as by antibody (Ab) affinity chromatography.

The method may result in the production of at least about 10, 20, 30, 40, 50, 100, 200, 500, 1,500, 2,000, 2,500, 3,000, 3,500, or 4,000 pg/mL of IL-16. In a particular aspect, the method may result in the production of at least about 2,000 pg/mL of IL-16. In some aspects, the method may result in the production of at least about 20,000, 40,000, 60,000, 80,000, or 100,000 pg/mL of IL-16.

### F. Compositions Comprising the NK Cell Lysate

The NK cell lysate as described herein can be included in a composition. In some aspects, the composition may include one or more NK cell lysates. For compositions comprising lysed NK cells, these compositions may be treated to remove intact NK cells, NK cell debris, and/or NK cell genomic DNA by any suitable method to produce an NK cell lysate lacking intact NK cells, NK cell debris, and/or NK cell genomic DNA. In some aspects, NK cell debris includes MHC class I and/or II, cell membranes, and/or organelles. For example, NK cell membranes can be removed from a composition comprising an NK cell lysate by centrifugation to produce a lysate (supernatant), which is free from cell membranes, such as by centrifugation of lysed NK cells for about 10 minutes, at about 3,000 g, at about 4 °C. Alternatively, compositions comprising the NK cell lysate can be cleared of intact NK cells, NK cell debris, and/or NK cell genomic DNA by means other than centrifugation. For example, filtration or treatment of cells with conA beads are alternate methods.

Also provided herein is a composition including one or more NK cell lysates and one or more NK cell exosomes. In some aspects, the composition includes the NK cell lysate as described herein. Exosomes, also known as extracellular vesicles, are lipid nanovesicles released by many cell types. In some aspects, exosomes may be vesicles comprising a membrane that encloses an internal space. They can mediate intercellular communication by transporting nucleic acids and proteins between cells. In some aspects, exosomes may be derived from cells (e.g., NK cells) by direct plasma membrane budding or by fusion of the late endosome with the plasma membrane. In some aspects, the exosomes may be derived from NK cells, such as NK-92/aNK cells, haNK cells, t-haNK cells, primary NK cells, KHYG-1 cells, or a variant thereof. In some aspects, exosomes may be derived from primary NK cells. In some aspects, the primary NK cells may be expanded with IL-2, IL-12, IL-15, IL-18, or a combination thereof. In some aspects, expanding the NK cells with IL-2, IL-12, IL-15, IL-18, or a combination thereof may produce memory-like cytokine enhanced NK cells (m-ceNK).

Exosomes may contain RNAs, mRNA, miRNAs, DNA, proteins, carbohydrates, lipids, or a combination thereof. In some aspects, exosomes may be employed as drug delivery vehicles. In some aspects, exosomes may contain a therapeutic agent. In some aspects, exosomes may be isolated from the producer cell, for example, based on their size, density, biochemical parameters, or a combination thereof. In some aspects, exosomes may range in size from about 20 to about 300 nanometers.

In some aspects, the NK cell exosomes and the NK cell lysate may each be derived from the same NK cells. Alternatively, in some aspects, the NK cell exosomes and the NK cell lysate may each be derived from different NK cells.

Similar to an NK cell lysate described herein, a composition comprising an NK cell lysate (and optionally one or more NK cell exosomes), in some aspects, may include one or more additional cytokines, agonist derivatives of the cytokines, fusion constructs comprising the cytokines, or a combination thereof. In some aspects, a fusion construct may include one or more cytokines and/or one or more agonist derivatives of the cytokines. As referred to herein, an agonist derivative of a cytokine, or a cytokine agonist, may refer to a compound or molecule that binds to and activates a receptor of said cytokine. For example, an “IL-15 agonist” may refer to a compound or molecule that binds to and activates the IL-15 receptor (“IL-15Rβγ”). In some aspects, the cytokine agonist may be an IL-15 agonist. In some aspects, an IL-15 agonist may be a superagonist complex, such as NAI. An exemplary IL-15 agonist may be selected from the list comprising IL-15, a fusion protein comprising IL-15, IL-15:IL-15Rα, and a fusion protein comprising IL-15:IL-15Rα. In some aspects, the one or more additional cytokines, agonist derivatives thereof, or fusion constructs comprised thereof may comprise IL-1α, IL-1β, IL-2, IL-4, IL-5, IL-6, IL-7, IL-10, IL-12p40, IL-12p70, IL-13, IL-15, IL-17, IL-18. INF-γ, GM-CSF, TNF-α, TNF-β, VEGF, IL-8, Eotaxin-3, MCP-1, MCP-4, MDC, MIP-1α, MIP-1β, or TARC. In certain aspects, the additional cytokine may be IL-15, an agonist derivative thereof, or a fusion construct comprised thereof. In some aspects, a fusion construct may include IL-15 and/or one or more agonist derivatives of IL-15.

In some aspects, the NK cells may be expanded to a density of about 1×10¹, about 1×10², about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, about 1×10¹⁴, or about 1×10¹⁵ cells/mL prior to exosome collection and/or lysis. In some aspects, the NK cells may be expanded to a density of about 1×10⁶ cells/mL prior to exosome collection and/or lysis. In some aspects, the NK cells may be expanded to a density of at least 1×10¹, at least 1×10², at least 1×10³, at least 1×10⁴, at least 1×10⁵, at least 1×10⁶, at least 1×10⁷, at least 1×10⁸, at least 1×10⁹, at least 1×10¹⁰, at least 1×10¹¹, at least 1×10¹², at least 1×10¹³, at least 1×10¹⁴, or at least 1×10¹⁵ cells/mL prior to exosome collection and/or lysis. In a particular aspect, the NK cells may be expanded to a density of at least 1×10⁶ cells/mL prior to exosome collection and/or lysis.

In some aspects, it may be beneficial to include one or more excipients in a composition. One of skill in the art would appreciate that the choice of any one excipient may influence the choice of any other excipient. For example, the choice of a particular excipient may preclude the use of one or more additional excipients because the combination of excipients would produce undesirable effects. One of skill in the art would be able to determine empirically which excipients, if any, to include in the formulations or compositions disclosed herein. Excipients may include, but are not limited to, co-solvents, solubilizing agents, buffers, pH adjusting agents, bulking agents, surfactants, encapsulating agents, tonicity-adjusting agents, stabilizing agents, protectants, and viscosity modifiers. In some aspects, it may be beneficial to include a pharmaceutically acceptable carrier.

In some aspects, it may be beneficial to include a solubilizing agent. Solubilizing agents may be useful for increasing the solubility of any of the components of the formulation or composition, including a therapeutically active agent disclosed herein or an excipient. The solubilizing agents described herein are not intended to constitute an exhaustive list, but are provided merely as exemplary solubilizing agents that may be used. In certain aspects, solubilizing agents include, but are not limited to, ethyl alcohol, tert-butyl alcohol, polyethylene glycol, glycerol, methylparaben, propylparaben, polyethylene glycol, polyvinyl pyrrolidone, and any pharmaceutically acceptable salts and/or combinations thereof.

The pH may be any pH that provides desirable properties for the composition. Desirable properties may include, for example, therapeutically active agent stability, increased therapeutically active agent retention as compared to compositions at other pHs, and improved filtration efficiency.

In some aspects, it may be beneficial to include a tonicity-adjusting agent. The tonicity of a liquid composition is an important consideration when administering the composition to a patient, for example, by parenteral administration. Tonicity-adjusting agents, thus, may be used to help make a composition suitable for administration. Tonicity-adjusting agents are well known in the art. Accordingly, the tonicity-adjusting agents described herein are not intended to constitute an exhaustive list but are provided merely as exemplary tonicity-adjusting agents that may be used. Tonicity-adjusting agents may be ionic or non-ionic and include, but are not limited to, inorganic salts, amino acids, carbohydrates, sugars, sugar alcohols, and carbohydrates. Exemplary inorganic salts may include sodium chloride, potassium chloride, sodium sulfate, and potassium sulfate. An exemplary amino acid is glycine. Exemplary sugars may include sugar alcohols such as glycerol, propylene glycol, glucose, sucrose, lactose, and mannitol.

In some aspects, it may be beneficial to include a stabilizing agent. Stabilizing agents help increase the stability of therapeutically active agents in compositions of the disclosure.

In some aspects, it may be beneficial to include a protectant. Protectants are agents that protect a pharmaceutically active ingredient (e.g., a therapeutically active agent as disclosed herein, such as IL-16) from an undesirable condition (e.g., instability caused by freezing or lyophilization, or oxidation). Protectants can include, for example, cryoprotectants, lyoprotectants, and antioxidants. Cryoprotectants are useful in preventing loss of potency of an active pharmaceutical ingredient (e.g., a therapeutically active agent as disclosed herein) when a formulation is exposed to a temperature below its freezing point. For example, a cryoprotectant could be included in a reconstituted lyophilized formulation so that the formulation could be frozen before dilution for intravenous (IV) administration. Cryoprotectants are well known in the art. Accordingly, the cryoprotectants described herein are not intended to constitute an exhaustive list, but are provided merely as exemplary cryoprotectants that may be used. Cryoprotectants include, but are not limited to, solvents, surfactants, encapsulating agents, stabilizing agents, viscosity modifiers, and combinations thereof. Cryoprotectants may include, for example, disaccharides (e.g., sucrose, lactose, maltose, and trehalose), polyols (e.g., glycerol, mannitol, sorbitol, and dulcitol), glycols (e.g., ethylene glycol, polyethylene glycol, propylene glycol).

Lyoprotectants are useful in stabilizing the components of a lyophilized formulation or composition. For example, a therapeutically active agent as disclosed herein could be lyophilized with a lyoprotectant prior to reconstitution. Lyoprotectants are well known in the art. Accordingly, the lyoprotectants described herein are not intended to constitute an exhaustive list, but are provided merely as exemplary lyoprotectants that may be used. Lyoprotectants include, but are not limited to, solvents, surfactants, encapsulating agents, stabilizing agents, viscosity modifiers, and combinations thereof. Exemplary lyoprotectants may be, for example, sugars and polyols, trehalose, sucrose, dextran, and hydroxypropylbeta-cyclodextrin are non-limiting examples of lyoprotectants.

Antioxidants are useful in preventing oxidation of the components of a composition. Oxidation may result in aggregation of a drug product or other detrimental effects to the purity of the drug product or its potency. Antioxidants are well known in the art. Accordingly, the antioxidants described herein are not intended to constitute an exhaustive list but are provided merely as exemplary antioxidants that may be used. Antioxidants may be, for example, sodium ascorbate, citrate, thiols, metabisulfite, and combinations thereof.

Similar to the NK cell lysate, a composition comprising the NK cell lysate may also be used in stimulating an immune response. Additionally or alternatively, the composition may be used in preventing and/or treating an infection. Additionally or alternatively, the composition may be used in preventing and/or treating cancer. Additionally or alternatively, the composition may be used in initiating, enhancing, or prolonging an anti-tumor response. For example, a composition comprising an NK cell lysate may include one or more adjuvants, one or more toll-like receptor (TLR) agonists, one or more co-stimulatory molecules, one or more bacillus Calmette-Guerins (BCGs), or a combination thereof. In some aspects, the composition may be a vaccine.

Compositions of the disclosure can further include any other compounds that are useful for protecting a subject from a particular disease or condition, including an infectious disease or cancer, or any compounds that treat or ameliorate any symptom of such a disease. In some aspects, compositions of the disclosure may be referred to as pharmaceutical compositions.

### G. Method for Preparing the Composition

A method of preparing a composition, for example for use in the treatment of cancer, is also provided herein. The method includes expanding NK cells in a cell culture to produce m-ceNK cells, collecting exosomes from the supernatant of m-ceNK cells, lysing the m-ceNK cells to produce a lysate, removing intact NK cells, NK cell debris, and NK cell genomic DNA from the lysate, and formulating an effective amount of the exosomes and an effective amount of the lysate for administration to a subject in need thereof. In some aspects, the NK cells may be primary human NK cells.

In some aspects, the cell culture may include one or more additional cytokines, agonist derivatives thereof, fusion constructs comprised thereof, or a combination thereof. In some aspects, the one or more additional cytokines, agonist derivatives of the cytokines, or fusion constructs comprising the cytokines and/or agonist derivative of the cytokine may be IL-1α, IL-1β, IL-2, IL-4, IL-5, IL-6, IL-7, IL-10, IL-12p40, IL-12p70, IL-13, IL-15, IL-17, IL-18, INF-γ, GM-CSF, TNF-α, TNF-β, VEGF, IL-8, Eotaxin-3, MCP-1, MCP-4, MDC, MIP-1α, MIP-1β, or TARC. In certain aspects, the additional cytokine may be IL-15 or an agonist derivative of IL-15. In certain aspects, the additional cytokine may be IL-2 or an agonist derivative of IL-2. In certain aspects, the additional cytokine may be IL-12 or an agonist derivative of IL-12. In certain aspects, the additional cytokine may be IL-18 or an agonist derivative of IL-18. In some aspects, the composition comprising an NK cell lysate may contain IL-15, IL-2, IL-12, and IL-18, an agonist derivative of IL-15, IL-2, IL-12, and/or IL-18, or a fusion construct comprised of IL-15, IL-2, IL-12, and/or IL-18 and/or an agonist derivative of IL-15, IL-2, IL-12, and/or IL-18. In some aspects, expanding primary human NK cells in a cell culture including IL-15 or an agonist derivative thereof, IL-2 or an agonist derivative thereof, IL-12 or an agonist derivative thereof, and IL-18 or an agonist derivative thereof may produce memory-like cytokine enhanced NK cells (m-ceNK).

In some aspects, lysing the m-ceNk cells to produce a lysate may include one or more cycles of sonicating the m-ceNK cells and/or one or more cycles of freezing and thawing the m-ceNK cells. In certain aspects, lysing the m-ceNK cells may include one or more cycles of freezing and thawing the m-ceNK cells. In certain aspects, lysing the m-ceNK cells may include three cycles of freezing and thawing the m-ceNK cells. In some aspects, the m-ceNK cells may be suspended in a solution, such as a salt solution, prior to lysing the m-ceNK cells. In some aspects, no detergent may be added during lysis. In some aspects, removing the intact NK cells, NK cell debris, and NK cell genomic data may include centrifugation.

In some aspects, the NK cells may be expanded to a density of about 1×10¹, about 1×10², about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, about 1×10¹⁴, or about 1×10¹⁵ cells/mL prior to exosome collection and/or lysis. In some aspects, the NK cells may be expanded to a density of about 1×10⁶ cells/mL prior to exosome collection and/or lysis. In some aspects, the NK cells may be expanded to a density of at least 1×10¹, at least 1×10², at least 1×10³, at least 1×10⁴, at least 1×10⁵, at least 1×10⁶, at least 1×10⁷, at least 1×10⁸, at least 1×10⁹, at least 1×10¹⁰, at least 1×10¹¹, at least 1×10¹², at least 1×10¹³, at least 1×10¹⁴, or at least 1×10¹⁵ cells/mL prior to exosome collection and/or lysis. In a particular aspect, the NK cells may be expanded to a density of at least 1×10⁶ cells/mL prior to exosome collection and/or lysis.

In some aspects, the composition prepared by the method described herein may be used to prevent, reduce the occurrence of, and/or treat cancer; prevent, reduce the occurrence of, and/or treat an infection; and/or initiate, enhance, or prolong an anti-tumor response.

### H. Methods of Use

In the methods disclosed herein, the NK cell lysate or composition comprising the NK cell lysate can be administered to animals, including any vertebrate, and particularly to any member of the Vertebrate class, Mammalia, including, without limitation, primates, rodents, livestock, and domestic pets. Livestock include mammals to be consumed or that produce useful products (e.g., sheep for wool production). Other examples of mammals include humans, dogs, cats, mice, rats, goats, sheep, cattle, horses, and pigs.

An “individual” is a vertebrate, such as a mammal, including without limitation a human. The term “individual” can be used interchangeably with the term “animal”, “subject”, or “patient”. In some aspects, the subject is human. In other aspects, the subject is a non-human animal, such as a canine or a feline.

The method of use may include administering the NK cell lysate or a composition comprising the NK cell lysate described herein. The method of use may be performed in vitro or in vivo.

Administration of a composition, such as the NK cell lysate or composition comprising the NK cell lysate, can be systemic, mucosal and/or proximal to the location of a target site (e.g., near a tumor). Suitable routes of administration will be apparent to those of skill in the art, depending on the type of condition to be prevented or treated, the therapeutically active agent used, and/or the target cell population or tissue. Various acceptable methods of administration include, but are not limited to, intravenous administration, intraperitoneal administration, intramuscular administration, intranodal administration, intracoronary administration, intraarterial administration (e.g., into a carotid artery), subcutaneous administration, retroorbital administration, transdermal delivery, intratracheal administration, subcutaneous administration, intraarticular administration, intraventricular administration, inhalation (e.g., aerosol), intracranial, intraspinal, intraocular, aural, intranasal, oral, pulmonary administration, impregnation of a catheter, and direct injection into a tissue. In one aspect, routes of administration include: intravenous, intraperitoneal, subcutaneous, intradermal, intranodal, intramuscular, transdermal, inhaled, intranasal, oral, intraocular, intraarticular, intracranial, and intraspinal. Parenteral delivery can include intradermal, intramuscular, intraperitoneal, intrapleural, intrapulmonary, intravenous, subcutaneous, atrial catheter and venal catheter routes. Aural delivery can include ear drops, intranasal delivery can include nose drops or intranasal injection, and intraocular delivery can include eye drops. Aerosol (inhalation/intranasal) delivery can also be performed using methods standard in the art. Other routes of administration that modulate mucosal immunity are useful in the treatment of viral infections. Such routes include bronchial, intradermal, intramuscular, intranasal, other inhalatory/intranasal, rectal, subcutaneous, topical, transdermal, vaginal and urethral routes. Intranasal formulations and intranasal administration of a composition can be prepared as discussed, for example, in Rebhun et al. Journal for Immunotherapy of Cancer vol. 10,6 (2022): e004493, which is hereby incorporated by reference in its entirety.

In some aspects, the NK cell lysate and/or composition comprising the NK cell lysate can be administered by injection. In a particular aspect, the injection may be an intratumoral injection. In some aspects, the injection may be an intravenous injection. In a further aspect, the NK cell lysate/composition may be injected into an infected abscess, such as an abscess infected by a bacteria, virus, parasite, or fungus. In some aspects, the NK cell lysate and/or composition may be for autologous administration to the subject. In some aspects, the NK cell lysate and/or composition may be for allogeneic administration to the subject.

Additionally or alternatively, the NK cell lysate/composition may be administered via instillation, also referred to as medical instillation. In some aspects, the NK cell lysate/composition may be instilled in a liquid form. In some aspects, a liquid form may be instilled drop by drop or with a catheter into a body space or cavity. In some aspects, drop by drop administration may be performed as eye drops, ear drops, or nose drops. In some aspects, catheter administration may be via a urinary catheter. In some aspects, the NK cell lysate/composition can be formulated in a pharmaceutically acceptable excipient suitable for administration by injection or instillation of a subject.

The present disclosure also provides a method for stimulating an immune response in a subject in need thereof. In some aspects, a method for stimulating an immune response may refer to stimulating an immune response against a disease target. A disease target may be those due to infection by pathogenic agents, cancers, and other disorders amenable to treatment by elicitation of an immune response. In some aspects, an antigen associated with a disease may be considered a disease target.

An immune response, as used herein, may refer to any response that employs the immune system. For example, an immune response may employ any immune system cell, such as a T cell, B cell, NK cell, monocyte, macrophage, neutrophil, eosinophil, basophil, mast cell, dendritic cell, etc. An immune response can be a physiological reaction that occurs within an organism (or outside an organism in in vitro methods) for the purpose of defending against exogenous factors. Exogenous factors may be a variety of different toxins, viruses, intra- and extracellular bacteria, protozoa, helminths, and fungi which could cause health problems. Immune responses may occur due to allergies, transplanted tissue, and Rh disease (i.e., hypersensitivities). In some aspects, the immune response may be an antitumor immune response (i.e., antitumor immunity). In some aspects, an immune response may be initiated by the innate and/or the adaptive immune system.

In some aspects, the NK cell lysate/composition described herein may stimulate an immune response. In some aspects, one or more immunomodulators in the NK cell lysate/composition may stimulate an immune response. In a certain aspect, IL-16 from the NK cell lysate/composition may stimulate an immune response. In some aspects, IL-16 may be extracted/isolated from the NK cell and/or NK cell lysate/composition. In some aspects, extracted IL-16 may stimulate an immune response. In some aspects, a combination of one or more immunomodulators in the NK cell lysate/composition may stimulate an immune response.

The disclosure provides a method for preventing, reducing the occurrence of, and/or treating an infection in a subject in need thereof. The method can include administering to the subject an effective amount of the NK cell lysate or composition comprising the NK cell lysate described herein.

“Effective amount” as used herein refers to the amount required to lessen or ameliorate the symptoms of a disease relative to an untreated subject. The effective amount of active agent used to practice methods of the present disclosure for therapeutic treatment of a disease may vary depending upon the manner of administration, the age, body weight, and general health of the subject. The attending physician or veterinarian may decide the appropriate amount and dosage regimen.

“Preventing” and “prevention” refer to the administration of an agent or composition to a clinically asymptomatic individual who may be susceptible or predisposed to a particular adverse disease of condition, and thus relates to the prevention of the occurrence of symptoms and/or their underlying cause.

“Treating” and “treatment” as used herein refer to the administration of an agent or composition to a clinically symptomatic individual afflicted with an adverse disease or condition, so as to effect a reduction in severity and/or frequency of symptoms, eliminate the symptoms and/or their underlying cause, and/or facilitate improvement or remediation of damage. It will be appreciated that, although not precluded, treating a disease or condition does not require that the disease, condition, or symptoms associated therewith be completely eliminated.

Method of Preventing, Reducing the Occurrence of and/or Treating an Infection

In some aspects, a method for preventing, reducing the occurrence of, and/or treating an infection in a subject in need thereof is provided. In some aspects, the NK cell lysate or composition comprising the NK cell lysate (NK composition) described herein may be used to prevent, reduce the occurrence of, and/or treat an infection.

In some aspects, the NK composition may include one or more exosomes. In some aspects, the one or more exosomes may be used to prevent, reduce the occurrence of, and/or treat an infection. In some aspects, one or more immunomodulators in the one or more exosomes may be used to prevent, reduce the occurrence of, and/or treat an infection. In some aspects, one or more immunomodulators in the NK cell lysate/composition may be used to prevent, reduce the occurrence of, and/or treat an infection. In some aspects, IL-15 from the NK cell lysate/composition may be used to prevent, reduce the occurrence of, and/or treat an infection. In a certain aspect, IL-16 from the NK cell lysate/composition may be used to prevent, reduce the occurrence of, and/or treat an infection. In some aspects, IL-16 may be extracted/isolated from the NK cell and/or NK cell lysate/composition, and the extracted IL-16 may be used to prevent, reduce the occurrence of, and/or treat an infection. In some aspects, a combination of one or more immunomodulators in the NK cell lysate/composition may be used to prevent, reduce the occurrence of, and/or treat an infection.

In some aspects, the infection may be bacterial, viral, fungal, or parasitic. Non-limiting example viral infections include human papillomavirus (HPV), Epstein-Barr virus (EBV), rhinovirus, coronavirus, adenovirus, norovirus, enterovirus, herpes simplex virus (HSV), and human immunodeficiency virus (HIV). Non-limiting example bacterial infections include bacterial meningitis, pneumonia, tuberculosis, gastritis, food poisoning, eye infections, skin infections, and sexually transmitted infections. Non-limiting example fungal infections include coccidioidomycosis, histoplasmosis, candidiasis, athlete's foot, ringworm, and some eye infections. Non-limiting example parasitic infections include malaria, toxoplasmosis, head lice, giardiasis, and pinworms.

In some aspects, the NK cell lysate/composition can treat an infection within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 46 or 48 hours after injection. In a particular aspect, the NK cell lysate/composition can treat an infection within 24 hours of injection. In some aspects, the NK cell lysate/composition may provide protection against infection reoccurrence.

Method for Preventing, Reducing the Occurrence of and/or Treating Cancer

The disclosure also provides a method for preventing, reducing the occurrence of, and/or treating cancer in a subject in need thereof. In some aspects, preventing and/or treating cancer includes preventing and/or treating a tumor and/or a neoplasm.

In some aspects, the NK cell lysate/composition described herein may be used to prevent and/or treat cancer. In some aspects, the NK composition may include one or more exosomes. In some aspects, the one or more exosomes may be used to prevent and/or treat cancer. In some aspects, one or more immunomodulators in the one or more exosomes may be used to prevent and/or treat cancer. In some aspects, one or more immunomodulators in the NK cell lysate/composition may be used to prevent and/or treat cancer. In some aspects, IL-15 from the NK cell lysate/composition may be used to prevent and/or treat cancer. In a certain aspect, IL-16 from the NK cell lysate/composition may be used to prevent and/or treat cancer. In some aspects, IL-16 may be extracted/isolated from the NK cells and/or NK cell lysate/composition, and the extracted IL-16 may be used to prevent and/or treat cancer. In some aspects, a combination of one or more immunomodulators in the NK cell lysate/composition may be used to prevent and/or treat cancer. In some aspects, the composition may prepared by the method of preparing a composition for use in the treatment of cancer as described herein.

Following administration, in some aspects, the NK cell lysate/composition can induce death in one or more tumor cells. In some aspects, the NK cell lysate/composition can induce tumor cell death within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 46 or 48 hours after injection. In a particular aspect, the NK cell lysate/composition can induce tumor cell death within 24 hours of injection. Additionally or alternatively, the NK cell lysate/composition may stop tumor cell proliferation at moderate and high concentrations. In some aspects, the NK cell lysate/composition may stop tumor cell proliferation when about 1-100 μL of NK cell lysate/composition is administered to a subject in a final injection volume of about 200 μL. For example, in some aspects, the NK cell lysate/composition may stop tumor cell proliferation when about 1, 5, 10, 15, 20, 25, 30, 35, 40, 45, 50, 55, 60, 65, 70, 75, 80, 85, 90, 95, or 100 μL of NK cell lysate/composition is administered to a subject in a final injection volume of about 200 μL. In some aspects, the NK cell lysate/composition may induce tumor cell death or stop tumor cell proliferation of MX-1, SKOV-3, A549, CTAC, DH-82, and K562 cells (sources in Examples). In some aspects, the NK cell lysate/composition can provide protection from tumor reoccurrence. In some aspects, the NK cell lysate/composition can provide protection from tumor reoccurrence of the tumor it was injected into. Additionally or alternatively, the NK cell lysate/composition can provide protection from reoccurrence of a distal tumor it was not injected into. For example, the NK cell lysate/composition may provide protection from reoccurrence of a tumor adjacent to the tumor it was injected into. In some aspects, the NK cell lysate/composition may cause regression of the tumor it was injected into. Additionally or alternatively, the NK cell lysate/composition may cause regression of one or more distal tumors the NK cell lysate/composition was not injected into. For example, the NK cell lysate/composition may cause regression of a tumor adjacent to the tumor it was injected into.

Following administration, in some aspects, the NK cell lysate/composition may not induce death in one or more non-cancerous cells. In other words, the NK cell lysate/composition may be selectively inducing anti-proliferative effects in cancer cells. In some aspects, the NK cell lysate/composition does not induce general toxicity (killing all cell types). In some aspects, the NK cell lysate/composition's anti-proliferative effects are cell-line specific.

Treatment of patients with a tumor/cancer may include any of the following: Adjuvant therapy (also called adjunct therapy or adjunctive therapy) to destroy residual tumor cells that may be present after the known tumor is removed by the initial therapy (e.g. surgery), thereby preventing possible cancer reoccurrence; neoadjuvant therapy given prior to the surgical procedure to shrink the cancer; induction therapy to cause a remission, typically for acute leukemia; consolidation therapy (also called intensification therapy) given once a remission is achieved to sustain the remission; maintenance therapy given in lower or less frequent doses to assist in prolonging a remission; first line therapy (also called standard therapy); second (or 3rd, 4th, etc.) line therapy (also called salvage therapy) is given if a disease has not responded or reoccurred after first line therapy; and palliative therapy (also called supportive therapy) to address symptom management without expecting to significantly reduce the cancer.

“Neoplasm” and “neoplastic” as used herein refers to an abnormal growth of cells. A neoplasm may be a small, benign (non-cancerous) growth such as a mole, or it may be a malignant (cancerous) or precancerous tumor.

“Tumor” as used herein refers to a mass of transformed cells that are characterized by neoplastic uncontrolled cell multiplication and at least in part, by containing angiogenic vasculature. The abnormal neoplastic cell growth is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to metastasize (i.e. a metastatic tumor), a tumor also can be nonmalignant (i.e. non-metastatic tumor). Tumors are hallmarks of cancer, a neoplastic disease, where the natural course of which is fatal.

In some aspects, the cancer may be a leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyo-sarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papil-lary carcinoma, papillary adenocarcinomas, cystadenocarci-noma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung car-cinoma, small cell lung carcinoma, bladder carcinoma, epithelial carcinoma, glioma, glioblastoma multiforme, astro-cytoma, medulloblastoma, craniopharyngioma, ependymoma, pinealoma, hemangioblastoma, acoustic neuroma, oligodendroglioma, schwannoma, meningioma, mela-noma, neuroblastoma, and retinoblastoma). In particular aspects, the neoplasia is multiple myeloma, beta-cell lymphoma, urothelial/bladder carcinoma or melanoma. In some aspect, the cancer and/or tumor may be an endocrine tumor, a gastrointestinal tumor, a genitourinary or gynecologic tumor, bladder cancer, breast cancer, head and neck tumor, hematopoietic tumor, skin tumor, thoracic or respiratory tumor, lymphoma, leukemia, ovary adenocarcinoma, lung carcinoma, breast cancer, thyroid cancer, malignant histiocytosis, and colon cancer. In a particular aspect, the cancer is bladder cancer. In a particular aspect, the NK cell lysate/composition may be instilled into a patient via a urinary catheter to prevent and/or treat bladder cancer.

### Method for Initiating, Enhancing, or Prolonging an Anti-Tumor Response

Along with methods of preventing, reducing the occurrence of, and/or treating infection or cancer, the disclosure provides a method for initiating, enhancing, or prolonging an anti-tumor response. An anti-tumor response may aid in the prevention and/or treatment of cancer.

An anti-tumor response may be referred to herein as an anti-tumor immune response or as anti-tumor immunity. An anti-tumor response may be any response that employs the immune system to prevent and/or treat a tumor and/or cancer. An anti-tumor response may refer to at least one positive therapeutic effect, such as for example, reduced number of cancer cells, reduced tumor size, reduced rate of cancer cell infiltration into peripheral organs, reduced rate of tumor metastasis or tumor growth, or progression free survival. In some aspects, an anti-tumor response may refer to an increase in an immune response against a tumor and/or cancer. In some aspects, an anti-tumor response may refer to inhibition and/or reduction in tumor growth.

Tumors can evade host immune surveillance by many different processes. These processes may be overcome by inactivation of proteins that are expressed by tumors. These proteins may be immunosuppressive, and examples include TGF-β, IL-10, and Fas ligand. In some aspects, the anti-tumor response may include triggering antigen presenting cells via MIC molecules. Anti-tumor immunity may additionally or alternatively include T cell priming against tumor antigens, trafficking anti-tumor T cells to tumor tissue, T cell infiltration of a tumor, and local activation to kill tumor cells. An anti-tumor response may also refer to resolution of the immune response, for example by clearing lysed tumor cells, re-establishment of normal tissue architecture, and/or homeostasis.

In some cases, a TGF-β trap is useful for increasing the effectiveness of NK cells and lysates/compositions thereof as an immunotherapy agent. In some aspects, the NK cells and lysates/compositions thereof comprise a TGF-β trap, and are therefore capable of binding to TGF-β. Thus, the NK cells and lysates/compositions thereof may be used to sequester TGF-β, and may be useful in methods for treating tumors. In some aspects, the TGF-beta trap is derived from the extracellular domain of human TGF-β receptor II (TGF-βRII). In some aspects, the TGF-beta trap comprises the TGF-β receptor II (TGF-βRII) molecule. In some aspects, the NK cell comprises a nucleic acid encoding the TGF-β trap, wherein transcription and translation of the nucleic acid results in expression of the TGF-β trap on the cell surface. In some aspects, the nucleic acid encoding the TGF-β trap is operably linked to a promoter, such as (without limitation), an inducible promoter. The TGF-β trap is discussed in detail, for example, in Reid, Tony R et al. Journal for Immunotherapy of Cancer vol. 12, 10 e009613. 26 Oct. 2024; Yi, Ming et al. Frontiers in Immunology vol. 13 1061394. 19 Dec. 2022; Guerra, Amaliris et al. American Journal of Hematology vol. 99.7 (2024): 1300-1312; Maron, Bradley A et al. American Journal of Respiratory and Critical Care Medicine vol. 203, 12 (2021): 1472-1487; and Gulley, James L et al. Molecular Oncology vol. 16, 11 (2022): 2117-2134.

In some aspects, an NK cell lysate/composition described herein may be used to initiate, enhance, or prolong an anti-tumor response. In some aspects, the NK composition may include one or more exosomes. In some aspects, the one or more exosomes may be used to initiate, enhance, or prolong an anti-tumor response. In some aspects, one or more immunomodulators in the one or more exosomes may be used to initiate, enhance, or prolong an anti-tumor response. In some aspects, one or more immunomodulators in the NK cell lysate/composition may be used to initiate, enhance, or prolong an anti-tumor response. In some aspects, IL-15 from the NK cell lysate/composition may be used to initiate, enhance, or prolong an anti-tumor response. In a certain aspect, IL-16 from an NK cell lysate/composition may be used to initiate, enhance, or prolong an anti-tumor response. In some aspects, IL-16 may be extracted/isolated from the NK cell and/or NK cell lysate/composition, and the extracted IL-16 may be used to initiate, enhance, or prolong an anti-tumor response. In some aspects, a combination of one or more immunomodulators in an NK cell lysate/composition may be used to initiate, enhance, or prolong an anti-tumor response.

The tumor and/or cancer may be any tumor and/or cancer described herein. In a particular aspect, the cancer is bladder cancer.

### I. Combination Therapy with an NK Cell Lysate/Composition

Also disclosed herein is a combination therapy using the NK cell lysate and/or composition comprising the NK cell lysate (optionally including one or more exosomes) described herein (also referred to herein as a first component) along with one or more additional components (also referred to herein as a second component).

“Co-administered” conveys simultaneous administration in the same formulation or in two different formulations via the same or different routes or sequential administration by the same or different routes. “Sequential” administration conveys a time difference of seconds, minutes, hours, or days between the administration of the two or more separate compounds.

In one aspect, one or more additional components are administered concurrently (i.e., co-administration) with the NK cell lysate/composition. In some aspects, a component is administered sequentially (i.e., sequential administration) with the NK cell lysate/composition. In another aspect, a component is administered before the NK cell lysate/composition. In another aspect, a component is administered after the NK cell lysate/composition. In some aspects, the NK cell lysate/composition and the additional component can be administered between 1, 2, 3, 4, 5, 6, 7, 8, 9, 10, 11, 12, 13, 14, 15, 16, 17, 18, 19, 20, or 21 days apart from each other. In one aspect, a component is administered in alternating doses with the NK cell lysate/composition, or in a protocol in which the NK cell lysate/composition is administered at prescribed intervals between or with one or more consecutive doses of a component, or vice versa. In one aspect, the NK cell lysate/composition is administered in one or more doses over a period of time prior to commencing the administration of a component. In other words, the NK cell lysate/composition is administered as a monotherapy for a period of time, and then the component administration is added, either concurrently with new doses of the NK cell lysate/composition, or in an alternating fashion with the NK cell lysate/composition. Alternatively, a component may be administered for a period of time prior to beginning administration of the NK cell lysate/composition. In one aspect, the NK cell and/or NK cell lysate/composition is engineered to express or carry an additional component, or a different cell/lysate/composition is engineered or produced to express or carry an additional component. In some aspects, the NK cell lysate/composition and additional component may be administered by different routes of administration.

As used herein with respect to administration of the NK cell lysate/composition or additional component, the term “concurrently” means to administer each of the NK cell lysate/composition and the additional component, and particularly the first dose of such lysate/composition and component, essentially at the same time or within the same dosing period, or within a time period during which the initial effects of preventing and/or treating cancer; initiating, enhancing, or prolonging an anti-tumor response; or stimulating an immune response. For clarity, concurrent administration does not require administration of both the NK cell lysate/composition and additional component at precisely the same moment, but rather, the administration of both the NK cell lysate/composition and additional component should occur within one scheduled dosing of the subject in order to prime the immune system and achieve the effect of the additional component concurrently (e.g., one component may be administered first, followed immediately or closely by the administration of the second component, and so on). In some circumstances, such as when both the NK cell lysate/composition and additional component are administered to the same site, the components may be provided in admixture, although even when administered at the same site, sequential administration of each component during the same dosing period may be used. In one aspect, the components are administered within the same 1-2 days, and in another aspect on the same day, and in another aspect within the same 12 hour period, and in another aspect within the same 8 hour period, and in another aspect within the same 4 hour period, and in another aspect within the same 1, 2 or 3 hour period, and in another aspect, within the same 1, 2, 3, 4, 6, 7, 8, 9, or 10 minutes.

In one aspect, the NK cell lysate/composition and additional component(s) are administered concurrently, but to different physical sites in a subject. For example, one component can be administered to one or more sites of a subject's body and the other component can be administered to one or more different sites of the subject's body, e.g., on different sides of the body or near different draining lymph nodes. In another aspect, the NK cell lysate/composition and additional component are administered concurrently and to the same or substantially adjacent sites in the patient. A substantially adjacent site is a site that is not precisely the same injection site to which the first composition or agent is administered, but that is in close proximity (is next to) the first injection site. In one aspect, the NK cell lysate/composition and additional component are administered in admixture. Some aspects may include combinations of administration approaches.

In some aspects, the additional component may be an agent, such as a therapeutically active agent. Examples of therapeutically effective agents include chemotherapeutic agents, therapeutic agents (e.g., cell therapies comprising whole/unlysed cells), targeted drugs, and immunomodulators. In some aspects, the additional immunomodulator may be IL-12, IL-15, IL-15:IL-15Rα or a stabilized derivative thereof, a tumor necrosis targeting (TNT) antibody (such as chTNT-3), or a combination thereof. In one aspect, the additional immunomodulator may be a IL-15:IL-15Rα or a stabilized derivative thereof, such as nogapendekin-alfa-inbakicep (NAI which is also referred to herein as “N-803”). In some aspects, the additional immunomodulator may be an immune checkpoint modulator, such as an anti-CTLA-4 antibody, an anti-PDL1 antibody, an anti-PD-1 antibody, an anti-TIM-3 antibody, an anti-SLAM-F7 antibody, or a combination thereof. In some aspects, the additional immunomodulator may include an effective amount of a yeast lysate. In some aspects, the additional immunomodulator may be another cytotoxic molecule, such as gasdermin D (GSDMD), perforin 1 (PRF1), granulysin (GNLY), a serine protease, such as a granzyme (e.g., human GzmA, GzmB, GzmH, GzmK and GzmM), or a cathepsin. In some aspects, the additional immunomodulator may be another cytokine. Thus, in some aspects, the NK cell lysate may include one or more additional cytokines, agonist derivatives of the cytokines, fusion construct comprising the cytokine and/or an agonist derivative of the cytokine, or a combination thereof. In some aspects, a fusion construct may include one or more cytokines and/or one or more agonist derivatives of the cytokines. As referred to herein, an agonist derivative of a cytokine, or a cytokine agonist, may refer to a compound or molecule that binds to and activates a receptor of said cytokine. For example, an “IL-15 agonist” may refer to a compound or molecule that binds to and activates the IL-15 receptor (“IL-15Rβγ”). In some aspects, the cytokine agonist may be an IL-15 agonist. In some aspects, an IL-15 agonist may be a superagonist complex, such as NAI. An exemplary IL-15 agonist may be selected from the list comprising IL-15, a fusion protein comprising IL-15, IL-15:IL-15Rα, and a fusion protein comprising IL-15:IL-15Rα. The additional cytokine, agonist derivative thereof, or fusion construct comprised thereof may be IFN-γ, IL-1α, IL-1β, IL-2, IL-4, IL-5, IL-6, IL-7, IL-10, IL-11, IL-12p40, IL-12p70, IL-13, IL-15, IL-17, IL-18, INF-γ, GM-CSF, G-CSF, TNF-α, TNF-β, VEGF, IL-8, Eotaxin-3, MCP-1, MCP-4, MDC, MIP-1α, MIP-1β, or TARC, in addition to IL-16.

In some aspects, the NK cell lysate/composition is administered with IFN-γ. As discussed in detail above, in some aspects, the NK cell lysate/composition comprises IL-16 that is endogenously produced by the NK cells. In some aspects, the NK cell lysate/composition and IFN-γ are both administered intratumorally, intranasally, or intravenously.

In some aspects, the NK cell lysate/composition is administered with a therapeutic agent comprising a cell therapy, for example, whole cell (i.e., not lysed) therapeutic. In many aspects, the whole cell therapeutic comprises unlysed NK-92/aNK cells, haNK cells, t-haNK cells, primary NK cells, KHYG-1 cells, m-ceNK cells, or a combination thereof. In some aspects, the NK cell lysate/composition is administered intratumorally or intranasally, and the whole cell therapeutic is administered intravenously.

A chemotherapeutic agent, as described herein, may also be referred to as a cytotoxic agent or a cytostatic drug, and can be used in chemotherapy. Chemotherapy may utilize cytotoxic agents to destroy or inhibit the growth and division of cells that divide rapidly (one of the properties of most cancer cells). Chemotherapeutic agents may be alkylating agents, anthracyclines, cytoskeletal disruptors (taxanes), epothilones, histone deacetylase inhibitors, inhibitors of topoisomerase I, inhibitors of topoisomerase II, kinase inhibitors, nucleotide analogs and precursor analogs, peptide antibiotics, platinum-based agents, retinoids, and vinca alkaloids and derivatives.

Examples of chemotherapeutic agents for combination with an NK cell lysate/composition provided herein include methotrexate (Rheumatrex®, Trexall®), 5-fluorouracil (commonly referred to as Adrucil®), vinorelbine (commonly referred to as Navelbine®), doxorubicin (commonly referred to as Adriamycin®), docetaxel (commonly referred to as Taxotere®), cyclophosphamide (commonly referred to as cyclophosphane), bleomycin (commonly referred to as Blenoxane), vinblastine (commonly referred to as Velban), dacarbazine (commonly referred to as DTIC-Dome), mustine (commonly referred to as chlormethine, mechlorethamine, Mustargen®), procarbazine (commonly referred to as Matulane®), prednisolone (commonly referred to as Orapred®, PediaPred®), vincristine (commonly referred to as Oncovin), cisplatin (commonly referred to as Platinol AQ), epirubicin (commonly referred to as Ellence®), capecitabine (commonly referred to as Xeloda®), folinic acid (commonly referred to as leucovorin), oxaliplatin (commonly referred to as Eloxatin®), gemcitabine (commonly referred to as Gemzar®), ifosfamide (commonly referred to as Ifex®), etoposide (commonly referred to as Toposar®, VePesid), paclitaxel (commonly referred to as Onxol, Taxol) and pemetrexed (commonly referred to as Alimta®). In certain aspects, the chemotherapeutic agent may be used to prevent and/or treat bladder cancer. In certain aspects, bladder cancer chemotherapeutic agents include docetaxel, paclitaxel, doxorubicin, methotrexate, ifosfamide, and pemetrexed.

As described above, an immunomodulator may refer to any substance that alters (e.g., induces, enhances, restores, suppresses, etc.) an immune response, or to an agent that utilizes or is derived from a component of the immune system. Immunomodulators for use in combination with the NK cell lysate/composition provided herein may be immunotherapies, corticosteroids, traditional disease-modifying antirheumatic drugs (DMARDs), biologics, and Janus kinase inhibitors.

Immunotherapy can help train an immune system to find infected cells, diseased cells, and/or cancer cells. In some aspects, immunotherapy can be cancer immunotherapy. Examples of immunotherapies include checkpoint modulators, adoptive cell therapy (e.g., T cell transfer therapy), monoclonal antibody therapy, cancer vaccines, and immune system modulators. Examples of immune system modulators include yeast lysate, cytotoxic molecules/proteins, cytokines, Bacillus Calmette-Guerin (BCG), and immunomodulatory drugs.

As described above, a yeast lysate may be prepared from a yeast that lacks yeast membranes and yeast cell walls, and it may contain one or more antigens that are endogenous or exogenous to the yeast.

As described above, a cytotoxic molecule or protein may refer to a molecule or protein that initiates and/or executes a cytotoxic effect. Examples of cytotoxic proteins include gasdermin D (GSDMD), perforin 1 (PRF1), granulysin (GNLY), serine proteases such as granzymes (e.g., human GzmA, GzmB, GzmH, GzmK and GzmM), and cathepsins.

As described above, a cytokine may refer to substances, such as chemokines, interferons (INs), interleukins (ILs), lymphokines, tumor necrosis factors (TNFs), hormones, and growth factors, which are secreted by immune system cells, and have an effect on other cells. In some aspects, a cytokine may be an interferon (INF) or an interleukin (IL). Examples of cytokines include IL-1α, IL-1β, IL-2, IL-4, IL-5, IL-6, IL-7, IL-10, IL-11, IL-12p40, IL-12p70, IL-13, IL-15, IL-16, IL-17, IL-18, INF-γ, GM-CSF, G-CSF, TNF-α, TNF-β, VEGF, IL-8, Eotaxin-3, MCP-1, MCP-4, MDC, MIP-1α, MIP-13, and TARC. Cytokines that are used to reduce side effects from cancer treatment may be referred to as hematopoietic growth factors. Examples of hematopoietic growth factors include erythropoietin, IL-11, granulocyte-macrophage colony-stimulating factor (GM-CSF), and granulocyte colony-stimulating factor (G-CSF).

BCG is known as a weakened form of the bacteria that causes tuberculosis. In some aspects, BCG may be used in combination with an NK cell lysate/composition herein to treat cancer, such as bladder cancer.

Immunomodulatory drugs may also be referred to as biological response modifiers. Examples of immunomodulatory drugs include thalidomide (Thalomid®), lenalidomide (Revlimid®), pomalidomide (Pomalyst®), and imiquimod (Aldara®, Zyclara®). In some aspects, thalidomide, lenaliodomide, and pomalidomide may cause cells to release IL-2. Thalidomide, lenaliodomide, and pomalidomide may also stop tumors from forming new blood vessels. Thalidomide, lenaliodomide, and pomalidomide may also be called angiogenesis inhibitors. In some aspects, Imiquimod causes cells to release cytokines.

As used herein, the term “checkpoint modulator” (also referred to as “immune checkpoint modulator”) refers to a molecule or to a compound that modulates (e.g., totally or partially reduces, inhibits, interferes with, activates, stimulates, increases, reinforces or supports) the function of one or more checkpoint molecules. Thus, a checkpoint modulator may be a “checkpoint inhibitor” (also referred to as “immune checkpoint inhibitor” or “inhibitor”) or a “checkpoint activator” (also referred to as “immune checkpoint activator” or “activator”). A “checkpoint inhibitor” totally or partially reduces, inhibits, interferes with, or negatively modulates the function of one or more checkpoint molecules. An “immune checkpoint activator” totally or partially activates, stimulates, increases, reinforces, supports or positively modulates the function of one or more checkpoint molecules. Immune checkpoint modulators are typically able to modulate (i) self-tolerance and/or (ii) the amplitude and/or the duration of the immune response. In some aspects, the immune checkpoint modulator used according to the present disclosure modulates the function of one or more human checkpoint molecules and is, thus, a “human checkpoint inhibitor”.

Checkpoint molecules are molecules, such as proteins, that are typically involved in immune pathways and, for example, regulate T-cell activation, T-cell proliferation and/or T-cell function. Accordingly, the function of checkpoint molecules, which is modulated (e.g., totally or partially reduced, inhibited, interfered with, activated, stimulated, increased, reinforced or supported) by checkpoint modulators, is typically the (regulation of) T-cell activation, T-cell proliferation and/or T cell function. Immune checkpoint molecules thus regulate and maintain self-tolerance and the duration and amplitude of physiological immune responses. Many of the immune checkpoint molecules belong to the B7:CD28 family or to the tumor necrosis factor receptor (TNFR) super family and, by the binding of specific ligands, activate signaling molecules that are recruited to the cytoplasmic domain.

Artificial T cell receptors (also known as chimeric T cell receptors, chimeric immunoreceptors, chimeric antigen receptors (CARs)) are engineered receptors, which graft an arbitrary specificity onto an immune effector cell. For example, the original NK-92 (aNK) cell line has been genetically modified to express various chimeric antigen receptors (CARs). haNK, t-haNK, and m-ceNK cells may also be modified to express various CARs. In some aspects, NK cells may be engineered to express PD-L1, CD19, EGFR, and/or HER2 specific CARs, for example. In a specific aspect, a T-haNK may be engineered to express PD-L1 specific CAR.

In some aspects, the immune checkpoint modulator may be an activator or an inhibitor of one or more immune checkpoint point molecule(s) selected from CD27, CD28, CD40, CD122, CD137, OX40, GITR, ICOS, A2AR, B7-H3, B7-H4, BTLA, CD40, CTLA-4, IDO, KIR, LAG3, PD-1, TIM-3, VISTA, CEACAMI, CARP, PS, CSF1R, CD94/NKG2 A, TDO, GITR, TNFR and/or FasR/DcR3; or an activator or an inhibitor of one or more ligands thereof.

In some aspects, the immune checkpoint modulator may be an activator of a (co-)stimulatory checkpoint molecule or an inhibitor of an inhibitory checkpoint molecule or a combination thereof. Accordingly, the immune checkpoint modulator may be (i) an activator of CD27, CD28, CD40, CD122, CD137, OX40, GITR and/or ICOS or (ii) an inhibitor of A2AR, B7-H3, B7-H4, BTLA, CD40, CTLA-4, IDO, KIR, LAG3, PD-1, PDL-1, PD-L2, TIM-3, VISTA, CEACAMI, CARP, PS, CSF1R, CD94/NKG2 A, TDO, TNFR and/or FasR/DcR3.

In some aspects, the immune checkpoint modulator may be an inhibitor of an inhibitory checkpoint molecule (but preferably no inhibitor of a stimulatory checkpoint molecule). Accordingly, the immune checkpoint modulator is even more preferably an inhibitor of A2AR, B7-H3, B7-H4, BTLA, CTLA-4, IDO, KIR, LAG3, PD-1, PDL-1, PD-L2, TIM-3, VISTA, CEACAMI, CARP, PS, CSF1R, CD94/NKG2 A, TDO, TNFR and/or DcR3 or of a ligand thereof.

In some aspects, the immune checkpoint modulator is an activator of a stimulatory or costimulatory checkpoint molecule (but preferably no activator of an inhibitory checkpoint molecule). Accordingly, the immune checkpoint modulator is more preferably an activator of CD27, CD28, CD40, CD122, CD137, OX40, GITR and/or ICOS or of a ligand thereof.

In some aspects, the immune checkpoint modulator is a modulator of the CD40 pathway, the IDO/TDO pathway, the CTLA-4 pathway and/or the PD-1/PD-LI pathway. In particular, the immune checkpoint modulator is preferably a modulator of CD40, CTLA-4, PD-LI, PD-L2, PD-1 and/or IDO/TDO, more preferably the immune checkpoint modulator is an inhibitor of CTLA-4, PD-LI, PD-L2, PD-1 and/or IDO/TDO or an activator of CD40, even more preferably the immune checkpoint modulator is an inhibitor of CTLA-4, PD-L I, PD-1 and/or IDO/TDO and most preferably the immune checkpoint modulator is an inhibitor of CTLA-4 and/or PD-1.

Corticosteroids may lessen an immune response and/or reduce inflammation in a subject. Some corticosteroids may be referred to as immunosuppressants, and may be used to treat autoimmune conditions, allergies, and cancers. Examples of corticosteroids include prednisone, prednisolone, and dexamethasone.

DMARDs may include medicines that lessen an immune response and/or decrease pain and inflammation. DMARDs may be used to treat rheumatoid arthritis, autoimmune conditions, allergies, skin conditions, and cancers. Some DMARDs may be used to prevent a subject's body from rejecting an organ following an organ transplant. Examples of DMARDs include Azathioprine (Imuran®), Cyclosporine (Gengraf® and Neoral®), Hydroxychloroquine (Plaquenil®), Leflunomide (Arava®), Methotrexate (Rheumatrex®, Trexall®), and Sulfasalazine (Azulfidine®).

Biologics may be referred to as a type of DMARD and may consist of engineered proteins. Biologics may target specific cells and pathways that cause inflammation. Examples of biologics include tumor necrosis factor (TNF) inhibitors, interleukin-1 (IL-1) inhibitors, interleukin-6 (TL-6) inhibitors, T-cell inhibitors, and B-cell inhibitors.

Examples of targeted drugs for combination with the NK cell lysate/composition provided herein include VEGF-targeted drugs, EGFR-targeted drugs, or antibody-drug-conjugates. Examples of VEGF-targeted drugs include Bevacizumab (Avastin(k), ramucirumab (Cyramza(k) or ziv-afiibercept (Zaltrap(k). Preferred examples of EGFR-targeted drugs include Cetuximab (Erbitux(k), paritumumab (Vectibix(k) or Regorafenib (Stivarga(k). Examples of antibody-drug conjugates include Ado-Trastuzumab emtansine (Kadcyla(k), SYD985, Trastuzumab vc-seco-DUBA, ABT-414, Depatuxizumab mafodotin, AMG 595, IMGN289, Laprituximab emtansine, ABBV-221, SGN-75, MDX-1203, BMS-936561, SGN-CD70 A, AMG 172, Gemtuzumab Ozogamicin (GO), SAR3419, coltuximab ravtansine, BAY 94-9343, anetumab ravtansine, Pinatuzumab vedotin, labetuzumab govitecan, Sacituzumab govitecan, MLN2704, Naratuximab emtansine, brentuximab vedotin, and Rovalpituzumab tesirine.

The NK cell lysate/composition may additionally or alternatively be combined with anti-inflammatory drugs such as betamethasone, non-steroid anti-inflammatory drugs (NSAIDs), acetaminophen, ibuprofen, naproxen, and/or other suitable drugs. Other tumor therapies such as radiotherapy, surgery, hormone therapy and/or immunotherapy. Thus, the provided methods can further include administering one or more additional therapeutic agents to the subject. Suitable additional therapeutic agents include, but are not limited to, analgesics, anesthetics, analeptics, corticosteroids, anticholinergic agents, anticholinesterases, anticonvulsants, antineoplastic agents, allosteric inhibitors, anabolic steroids, antirheumatic agents, psychotherapeutic agents, neural blocking agents, anti-inflammatory agents, antihelmintics, antibiotics, anticoagulants, antifungals, antihistamines, antimuscarinic agents, antimycobacterial agents, antiprotozoal agents, antiviral agents, dopamineigics, hematological agents, immunological agents, muscarinics, protease inhibitors, vitamins, growth factors, cell therapies, and hormones. The choice of agent and dosage can be determined readily by one of skill in the art based on the given disease or condition being treated.

In some aspects, combination therapy can stimulate an immune response. Additionally or alternatively, combination therapy can prevent and/or treat an infection and/or cancer in a subject in need thereof, by administering the NK cell lysate/composition described herein, and an additional component. The tumor and/or cancer may be any tumor and/or cancer described herein. In a particular aspect, the cancer is bladder cancer. Additionally or alternatively, combination therapy can initiate, enhance, and/or prolong an anti-tumor response.

### J. Vaccine Comprising the NK Cell Lysate/Composition

A vaccine is described herein that comprises the NK cell lysate and/or composition comprising the NK cell lysate.

As used herein, the terms “vaccination” or “immunization” refer to the elicitation (induction) of an immune response against a disease or condition. In some aspects, an immune response may be elicited against a disease target such as an antigen or immunogenic portion thereof, as a result of administration of the antigen, alone or together with an adjuvant. Vaccination may result in a protective and/or therapeutic effect, wherein subsequent exposure to the antigen (or a source of the antigen) elicits an immune response against the antigen (or source) that reduces or prevents a disease or condition in the host. The immune response that is elicited by administration of an immunotherapeutic composition (vaccine) can be any detectable change in any facet of the immune response (e.g., cell-mediated response, humoral response, cytokine production), as compared to in the absence of the administration of the composition.

In some aspects, the NK cell lysate/composition described herein may be used to induce a vaccine-like effect. In some aspects, one or more immunomodulators in the NK cell lysate/composition may be used to induce a vaccine-like effect. In a certain aspect, IL-16 from the NK cell lysate/composition may be used to induce a vaccine-like effect. In some aspects, IL-16 may be extracted/isolated from the NK cell and/or NK cell lysate/composition, and the extracted IL-16 may be used to induce a vaccine-like effect. In some aspects, a combination of one or more immunomodulators in the NK cell lysate/composition may be used to induce a vaccine-like effect.

A vaccine-like effect, as used herein, may refer to an immune response generated by the NK cell lysate/composition described herein, or by one or more immunomodulators in the NK cell lysate/composition described herein. For example, in some aspects, the NK cell lysate/composition may include T-haNK cell lysate, such as CD19 T-haNK cell lysate, for immunization against CD19 expressing B cell lymphoma. In some aspects, a vaccine-like effect may occur in response to the NK cell lysate/composition or a component thereof. The NK cell lysate/composition, or component thereof, such as IL-16, may induce a vaccine-like effect by inducing an immune response. In some aspects, the vaccine can provide protection against tumor reoccurrence. A vaccine-like effect may include preventing and/or treating a disease or condition, such as an infection or cancer.

### K. Kit Comprising the NK Cell Lysate/Composition

Also disclosed is a kit including the NK cell lysate and/or a composition comprising the NK cell lysate as described herein, and/or any of the individual components of the compositions described herein. Reagents may be present in free form or immobilized to a substrate such as a plastic dish, microarray plate, a test tube, a test rod, etc. The kit can also include suitable reagents for the detection of the reagent and/or for labeling of positive and negative controls, wash solutions, dilution buffers and the like. The kit can also include a set of written instructions for using the kit and/or interpreting the results. Kits may be prepared and used for any clinical, research, or diagnostic method.

The kit may include a package insert or label with directions for preventing and/or treating a disease or condition, such as cancer, by using the NK cell lysate/composition. In some aspects, the kit may be used for stimulating an immune response. The kit may additionally or alternatively be used for preventing and/or treating an infection. The kit may additionally or alternatively be used for preventing and/or treating cancer. The kit may additionally or alternatively be used for initiating, enhancing, and/or prolonging an anti-tumor response.

### L. NK Cell IL-16 Knockout

An NK cell with an IL-16 knock-out is disclosed. A gene knock out may also be referred to as gene deletion or gene inactivation. The IL-16 gene may be knocked out of an NK cell via methods known in the art. For example, IL-16 can be knocked out via genetic engineering techniques such as homologous recombination, CRISPR-Cas9, TALENs, siRNA, etc.

NK cells with an IL-16 knockout may be used to generate an NK cell lysate, or a composition comprising an NK cell lysate. An IL-16 knockout lysate/composition may be used with methods described herein as a control or reference. For example, an NK cell lysate/composition, as described herein that does not have an IL-16 knockout, may be compared to an NK cell lysate/composition that does have an IL-16 knockout. NK cell lysates/compositions may be compared based on their ability to stimulate and immune response, prevent and/or treat an infection, prevent and/or treat cancer, and/or initiate, enhance, or prolong an anti-tumor response.

### EXAMPLES

The following examples are merely illustrative, and do not limit this disclosure in any way.

### Example 1: NK Cell Lysate Preparation and Characterization

### Cell Lines and Cultures

NK cell lysates were obtained from aNK (NK-92) (Gong et al., 1994) and haNK cells (Jochems et al., 2016). haNK cells represent aNK cells that have been transduced with erIL-2 to allow them to grow and expand independent of IL-2. KHYG-1 cells were obtained from the German Collection of Microorganisms and Cell Cultures GmbH (Leibniz Institute DSMZ). aNK and haNK cell lines were cultured in medium that is used for ongoing clinical trials with these cell lines (X-Vivo™ 10 supplemented with 5% Human Male AB serum, heat-inactivated (Access Biologicals; Vista, CA) (HS) and 500 units/mL IL-2 for aNK but no additional IL-2 for haNK). KHYG-1 cells were cultured in Roswell Park Memorial Institute (RPMI) 1640 GlutaMAX™ (Gibco™, Grand Island, NY) supplemented with 10% fetal bovine serum (Gibco™). KHYG-1 cells used for the study of NK cell cytotoxicity are discussed in Suck et al. Experimental Hematology vol. 33, 10 (2005): 1160-71.

The following human cell lines were used as target cells for the different NK cell lysate preparations: K562 (chronic myeloid leukemia) American Type Culture Collection (ATCC®), CCL-243™; Cellonco™ MX-1 (human breast cancer) Creative Biolabs, #IOC-ZP248; SKOV-3 (human ovary adenocarcinoma), ATCC®, HTB-77™; A549 (human lung carcinoma), ATCC®, CCL-185™; SKBR-3 (human breast adenocarcinoma, HTB-30) ATCC®); Primary human lung fibroblasts, Sigma-Aldrich; and umbilical vein endothelial cells (HUVEC), Lonza. K562, SKOV-3, HTB-77™; A549, CCL-185™ cells were cultured in RPMI 1640 GlutaMAX™ (Gibco™, Grand Island, NY) supplemented with 10% fetal bovine serum (Gibco™). SKBR-3 and SKOV-3 cells were cultured in McCoys' 5 A (Corning, Corning NY). Human lung fibroblasts and HUVECs were cultured in fibroblasts and endothelial growth medium from Cell Applications (San Diego, CA). Culture medium for those cell lines included 10% Fetal Bovine Serum (Gibco).

The following canine cell lines were used: CTAC (thyroid carcinoma) MilliporeSigma, #90071812, cultured in RPMI/10% FC; DH-82 (malignant histiocytosis) ATCC®, CRL-3590™ cultured in Minimal Essential Medium (MEM)/10% fetal calf serum (FCS). OSCA-40 (canine osteosarcoma) Kerafast Inc., cultured in DMEM high glucose GlutaMAX™ (Gibco) with 10 mM HEPES supplement without Sodium Pyruvate.

Nuclear localized red (NucLight™ Red) fluorescent-labeled versions of the SKBR-3, SKOV-3, CTAC and OSCA-40 cell lines were generated by transduction with Incucyte® NucLight™ Red Lentivirus (Sartorius) in the presence of 8 g/mL polybrene, followed by selection with 1.5 to 2.5 g/mL puromycin (Invitrogen, Carlsbad, CA). For in vivo experiments, the murine MC-38 cell line (a murine colorectal cancer cell line) was grown in DMEM supplemented with 10% FBS (Hyclone), HEPES, 1% Penicillin/Streptomycin, and non-essential amino acids (Gibco).

### NK Cell Lysate Generation

To obtain NK cell lysate, aNK, haNK, and KHYG-1 cells were expanded at a density of 1×10⁶ cells/mL, harvested by centrifugation (e.g., 500×g for 5 minutes), and the cell pellets were washed with phosphate-buffered saline (PBS). In some cases, the PBX was without Ca2+/Mg2+. The cells were resuspended at 10⁸ cells/mL in a Balanced Salt Solution (BSS) cell lysis buffer (BSS: 150-250 mM NaCl, 50 mM Tris-HCl, pH 7.4) and lysed by either: a) 3 cycles of freeze (in liquid nitrogen for 30 seconds) and thaw (e.g., quick thaw for about 2 minutes in a 37 °C water bath) or b) 3 rounds of sonication at 20 kHz for 5 seconds each. No detergent was added to prevent proteins in the lysate from denaturing. Since the perforin/granzyme pathway is Ca++ dependent, no EDTA was added. The lysate was centrifuged at 3,000 g, at 4 °C, for 10 minutes to remove intact cells, insoluble cell debris and genomic DNA. The supernatant (the “NK cell lysate”) was collected and used either fresh or from cryopreserved (minus 80 °C) stock.

### Effect of aNK Cell Lysate on K562 Cells

Flow cytometric/fluorescence-activated cell sorting (FACS) cytotoxicity assays were used to identify the effect of aNK cell lysate on K562 cells. Target K562 cells were stained using the membrane dye PKH-67GL (Sigma-Aldrich®, St. Louis, MO) and incubated with aNK cell lysate for 30 min (Experiment 1) or 2 hrs (Experiment 2) at 37 °C. Dead cells were stained with propidium iodide (PI) (Molecular Probes, Eugene, OR) and samples were analyzed by flow cytometry. Percentage of dead cells was determined by the percentage of PI positive cells within the PKH-67GL positive target cell population. % Killing was calculated as follows=[% dead target cells in sample−% spontaneous dead target cells]/[100−% spontaneous dead target cells].

### Experiment 1: ANK Cell Lysate Causes Membrane Damage of K562 Cells

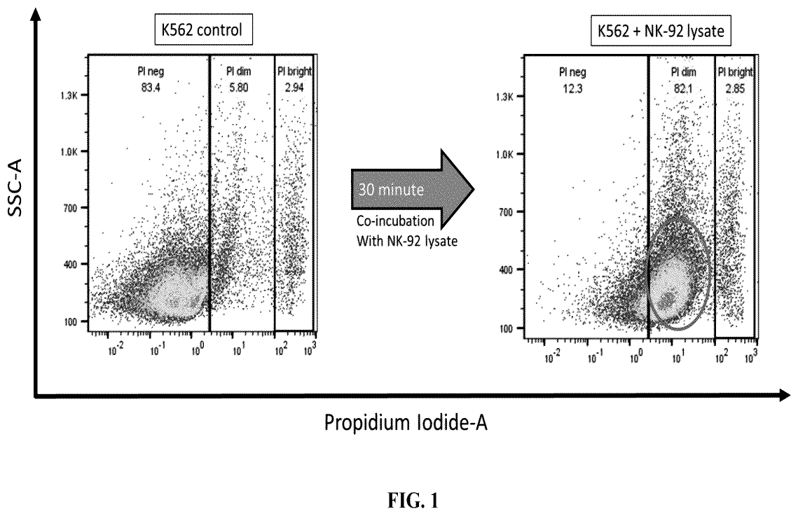

K562 cells were incubated with freshly prepared aNK cell lysate (generated by sonication at 20 Khz for 20 seconds for 4 cycles) in a FACS-based assay, using PI as a live/dead cell stain as described above. Activity of the aNK cell lysate caused intake of PI that caused a slight “shift” of the PI^{(neg)} K562 population into the PI^{(dim)} gate (FIG. 1). Thus, there is a mean fluorescence intensity (MFI) of 10¹, rather than 10³ (dead/dying cells). The shift of the K562 cells into the PI^{(dim)} gate indicates that the cell membrane is compromised/damaged, allowing for PI to enter the cell, but not stain the gDNA PI^{(bright)} in the intact nucleus. Thus, the aNK cell lysate can damage K562 cell membranes.

### Experiment 2: ANK Cell Lysate Causes More Membrane Damage of K562 Cells Using Freeze-Thaw Method

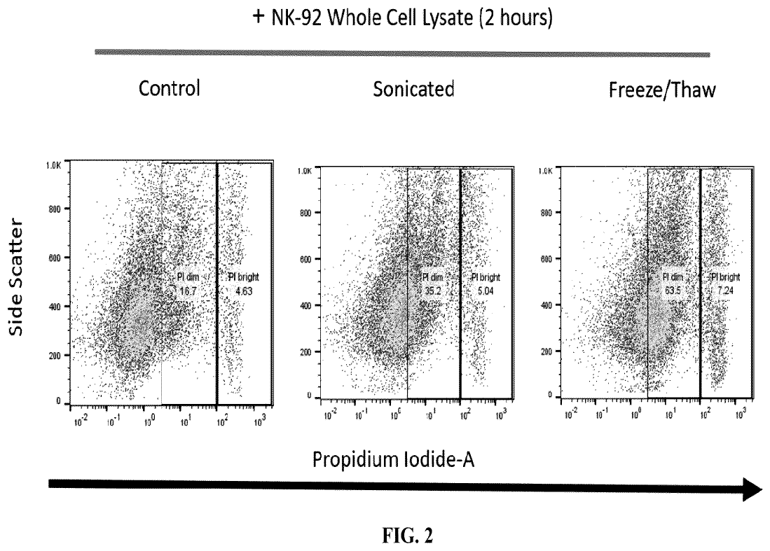

To obtain information on which aNK lysate preparation would affect target cells more profoundly, the aNK cells were treated by two different methods to generate the aNK cell lysate: 1) sonication cycles and 2) freeze-thaw cycles. This was a 2 hour assay to determine which method would be superior in causing membrane damage as measured by leakage of PI. The aNK cell lysate generated by three cycles of sonication caused a slight shift of the K562 cells into the PI^{(dim)} gate, while the aNK cell lysate generated by three cycles of freeze-thaw caused a greater shift of the K562 cells into the PI^{(dim)} gate (FIG. 2). Thus, aNK cell lysate generated by the freeze-thaw method resulted in more K526 cell membrane damage than aNK cell lysate generated by the sonication method.

### Identification of Proteins in NK Cell Lysate (Immunoblot Analysis)

Immunoblot (western blot) analysis SDS-PAGE was performed from cryopreserved aNK, haNK, and KHYG-1 cell lysate prepared by the freeze-thaw method. Protein concentration was measured at 562 nm using the bicinchoninic acid assay kit (Pierce™) and 40 g of protein was mixed with 4×NuPAGE™ LDS sample buffer supplemented with 10× NuPAGE™ reducing agent. All samples were denatured at 70 °C for 10 min before being added to a 4-12% polyacrylamide Bis-Tris gel (Invitrogen™) and electrophoresed at 200 v for 70 min in 2-(N-morpholino)ethanesulfonic acid (MES) SDS Running buffer (Invitrogen™). Following electrophoresis, the gel was incubated in 20% ethanol for 10 min and proteins were transferred onto polyvinylidene fluoride (PVDF) membrane using the iBlot™ 2 Dry Blotting system (Invitrogen™) according to manufacturer's instructions. Membranes were blocked in 5% dry milk in Tween®-Tris-buffered saline (TTBS) (50 mM Tris, 0.5 M NaCl, 0.05% Tween® 20, pH 7.4) for 1 hr at room temperature. The PVDF membrane was probed with primary (rabbit IgG) antibodies for the following granzymes, all of which were obtained from Cell Signaling Technology® (Danvers, MA) and used at 1:1000 dilution: GzmA (Catalog #4928S), GzmB (clone D6E9W, Catalog #46890S), GzmH (clone E4T2E, Catalog #18268S), GzmK (clone E9130, Catalog #47465S), GzmM (clone E7B4W, catalog #37765S), Perforin-1 (clone E7D8R, catalog #62550S). Granulysin mouse IgG2a (clone F-9, sc-271119) and Rab5b was obtained from Santa Cruz Biotechnology, CA and Vinculin-1 (clone E1E9V, 13901S) as a loading control was obtained from Cell Signaling Technology® (Danvers, MA). Antibodies against TSG101, Alix, HSP70, CD63, CD81 and Flotillin-1 were obtained from Cell Signaling Technology. Goat anti-mouse or Goat anti-rabbit secondary antibodies were conjugated with HRP (Cell Signaling, 1:10,000, and 1:5,000 respectively). The membrane was incubated with Amersham™ ECL™ Immunoblots that were developed by using chemiluminescence (SuperSignal™; Pierce, Rockford, IL, USA) and images were subsequently acquired using the Li-Cor® C-DiGit® Blot Scanner and Image Studio™ software.

The activated and pro-form fragments of IL-16 were detected using an antibody against the IL-16 C-terminus peptide antigen (R&D Systems). Total protein was extracted from cultured cells using radioimmunoprecipitation assay (RIPA) SDS extraction buffer, followed by 15 min at 4 °C, and then centrifugation at 20,000 g, for 20 min, at 4 °C. The supernatant was collected, and protein content quantified by bicinchoninic acid assay (BCA) before being submitted to immunoblot analysis. The PVDF membrane was probed with 1 μg/mL of goat anti-human IL-16 C-terminal peptide antigen affinity-purified polyclonal antibody (R&D Systems Catalog #AF-316-SP) followed by RP-conjugated anti-goat IgG secondary antibody (R&D Systems Catalog #HAF017). Protein bands were detected by enhanced chemiluminescence (ECL, as discussed in the preceding paragraph).

### Quantification of Cytokines and Chemokines in Cell Lysate

Quantification of a panel of cytokines and chemokines was performed by Meso Scale Diagnostics (MSD) Multi-Spot Assay System (Rockville, MD) using the V-Plex Plus Cytokine Panel 1 Human kit and Proinflammatory Panel 1 Human kit, according to manufacturer's instructions. Briefly, each MSD kit contains a 96 well-plate with microelectrode at the bottom of each well to enable it to produce high performance electrochemiluminescence with addition of MSD Read Buffer. These plates are pre-coated with either cytokine Panel 1 or Proinflammatory Panel 1 capture antibodies specific to the biomarkers on independent and well-defined spots. Readout is done using MSD Quick Plex SQ 120 Reader using Methodical Mind software.

### Quantification of Granzyme B Activity in Cell Lysate

Granzyme B enzymatic activity in cryopreserved and freshly prepared NK-92 and haNK cell lysates was determined with a Granzyme B Activity Assay Kit (Sigma-Aldrich) according to the manufacturer's instructions using a fluorimeter (SpectraMAX i3x, Molecular Devices, San Jose, CA) at wave lengths of 380 nm (excitation) and 500 nm (emission).

### Experiment 3: Perforin and Granzymes are Present in Lysate Preparations from NK-92 and haNK Cells

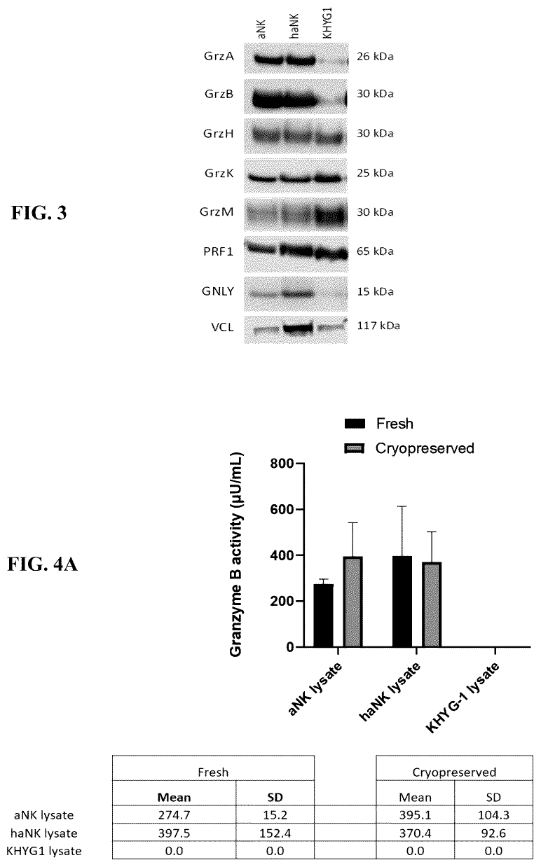

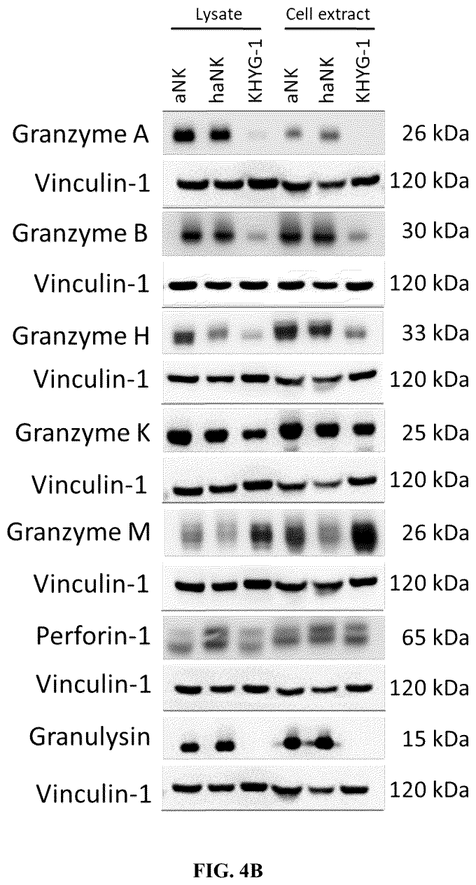

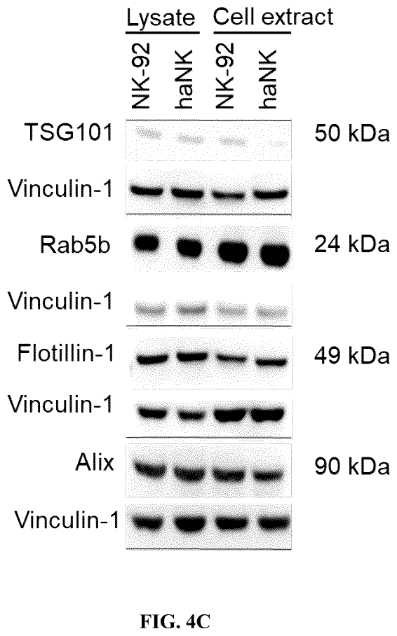

Perforin and granzymes are proteases that initiate and execute the cytotoxic effect of NK cells against cancer cells. Perforin and all five human granzymes (GzmA, GzmB, GzmH, GzmK and GzmM), could be identified by Western blot in lysate of aNK, haNK cells, and to variable degree in KHYG-1 cells (FIG. 3). The analysis also confirms that GzmB is the most abundant granzyme in aNK and haNK cells. In quantifying the amount of GzmB by ELISA, it was confirmed that (i) aNK and haNK cells have about the same concentration of the protease and (ii) that there is no significant difference in the concentration of GzmB between fresh and cryopreserved NK cell lysate (FIG. 4A).

To preserve both enzymatic activities and biological compatibility, a buffer solution was designed for lysate preparation—the buffer solution does not contain any detergent, chelating agents, or protease inhibitors. In order to determine how much of the cellular content was solubilized during cell lysate preparation, for comparison, whole cell extracts were prepared using a conventional denaturing lysis buffer.

Immunoblot analyses did not show any qualitative difference between our cell lysates and the denatured cellular extracts. Perforin and granzymes were the main effector proteins that initiate and execute the cytotoxic and anti-proliferative effect of NK cells against cancer cells. Perforin, granulysin, and all five granzymes (GzmA, GzmB, GzmH, GzmK and GzmM) were identified by Immunoblot in lysates of NK-92 and haNK cells (FIG. 4B).

Since it was likely that exosomes and multivesicular bodies were present in the lysate, Immunoblot was also used to probe for exosomal markers. As shown in FIG. 4C, the presence of TSG101, Rab5b, Flotillin-1 and Alix were confirmed in both NK-92 and haNK cell lysates. In contrast, CD81, HSP70 and CD63 were not detected (not shown).

Thus, the aNK and haNK cell-derived lysates and extracts contain perforin and the full spectrum of granzymes, and aNK and haNK cells produce significantly higher concentrations of lytic enzymes compared to KHYG-1 cells.

### Characterization of Cytokines in NK Cell Lysate

Quantification of human cytokines in the NK (aNK, haNK, and KHYG-1) cell lysate preparations (derived by freeze-thaw method) was performed by ELISA for IL-2 (BMS221INST, Invitrogen), IL-16 (EH259RB, Invitrogen), and IFN-g ELISA Kit (KHC4021, Invitrogen), according to manufacturer's instructions. To further characterize and quantify a broader cytokine and chemokine profile in the NK cell lysate, the MSD® MULTI-SPOT® Assay System from Meso Scale Diagnostics® (Rockville, MD) with V-Plex® Plus Cytokine Panel 1 (K15049G and K15050G) was used according to manufacturer's instructions. All statistical analyses were performed using GraphPad Prism (Version 10.1.1) software. Statistical differences for in vitro assays were determined using an unpaired, two-tailed Student's t-test.

### Experiment 4: Cytokine and Chemokine Levels in NK-92 and haNK Cells

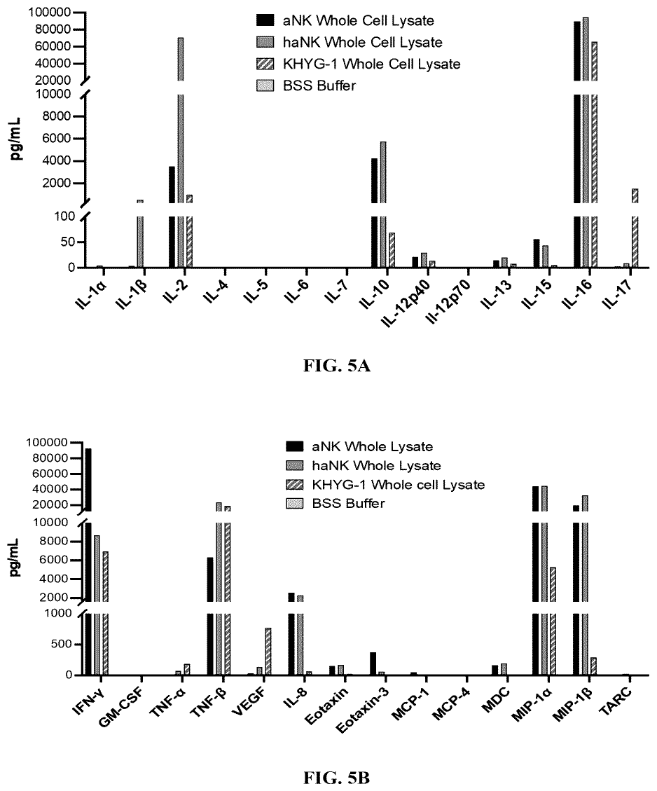

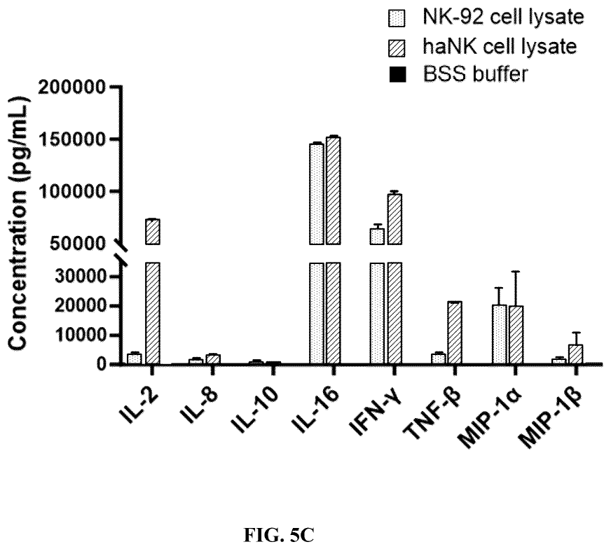

To determine the spectrum of cytokines and chemokines in the lysate from the three NK cell lines, the MSD® QuickPlex® SQ 120 reader from Meso Scale Discovery® was used. Results confirm significantly elevated cytokine concentrations of IL-2 IL-8, IL-10, IL-16, IFN-g (IFN-γ), TNF-b (TNF-β), MIP-1a (MIP1α) and MIP-1b (MIP1β) in both aNK and haNK cell lysates (FIG. 5A, FIG. 5B and FIG. 5C). Significantly lower concentrations were measured in KHYG-1 cell lysate, notably for the immune-active cytokines IL-2, IFN-g, and MIP-1 (FIG. 5A and FIG. 5B). Thus, aNK and haNK cell lysate includes significantly higher concentrations of cytokines/chemokines compared to KHYG-1 cell lysate. As expected, elevated concentrations of IL-2 were found in the lysate of haNK cells that had been engineered with the erIL-2 gene. The IL-2 detected in NK-92 lysate is likely from residual exogenous IL-2 in the culture medium which contains IL-2 to stimulate cell expansion. No measurable concentrations or very low concentrations were detected in aNK and haNK cell lysate for: IL-1α, IL-4, IL-5, IL-6, IL-7, IL-12, IL-13, IL-15, IL-17, GM-CSF, TNF-α, VEGF, Eotaxin, Eotaxin-3, MCP-1, MCP-4, MDC, and TARC.

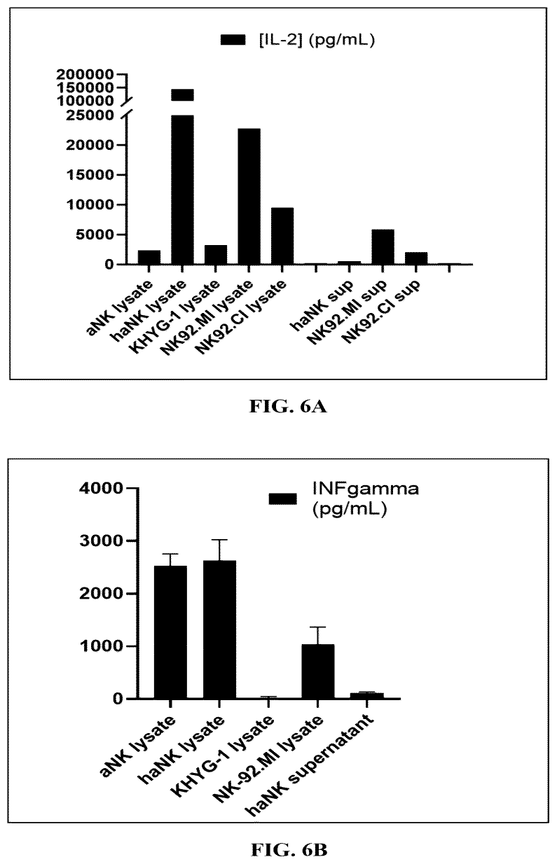

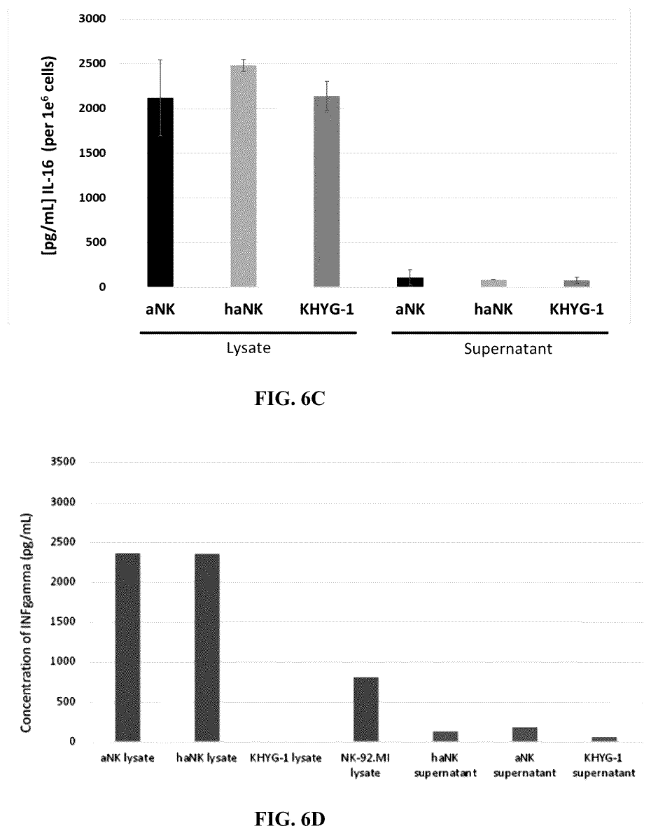

To confirm and further quantify the results obtained with the Meso Scale Discovery® platform for IL-2 and INF-g in aNK, haNK, and KHYG-1 lysates and extracts (“sup”), an ELISA was performed. Both cytokines were measured in significant amounts (FIG. 6A) whereas the lysate of KHYG-1 showed low concentration for IL-2 and barely measurable amounts for IFN-g. (FIG. 6B). IL-16 was also measured using ELISA and was detected in high amounts in aNK, haNK, and KHYG-t lysates but not in their extracts (“sup” or “supernatant”) (FIG. 6C and FIG. 6D).

The IL-2 in aNK cell lysate is likely due to the fact that the cells were expanded for several days in IL-2 before the aNK cell lysate was generated and the cells had retained IL-2 intracellularly. It is known that aNK cells do not transcribe IL-2. As expected, significant concentrations of IL-2 were found in the lysate of haNK cells which have been engineered with the erIL-2 gene to provide intracellular IL-2 sufficient to maintain proliferation and cytotoxicity without the need for exogenous IL-2.

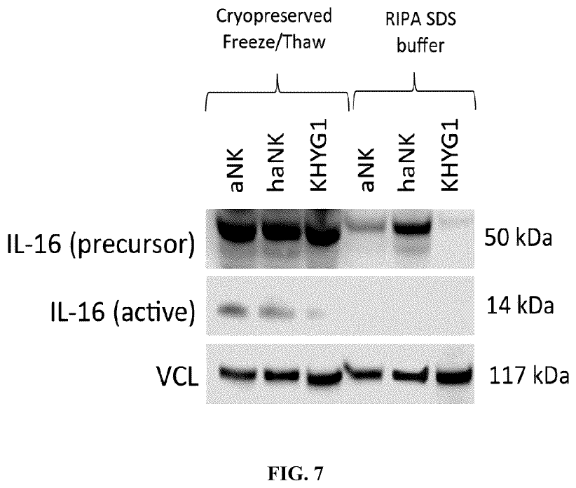

The observation of significant IL-16 concentration in the lysate of aNK and haNK cells was unexpected. To further define whether this represents the precursor peptide or the biologically active form of the IL-16 cytokine, a western blot was performed. It confirmed that the cryopreserved lysate had the active form of IL-16, which was not present in cell extracts prepared from detergent-based (RIPA SDS buffer) lysed cells (FIG. 7).

### Example 2: HaNK Cell Lysate Inhibits Target Cell Proliferation/Induces Cell Death

### Experiment 1: Live Target Cell Analysis

Nuclear localized red fluorescent-labeled target human (MX-1, SKOV-3, A549), and canine cancer cell lines (CTAC, DH-82) were plated in a 96-well plate with a seeding density of 1,000-2,000 cells per well or 1,000-3,000 cells per well, in 100 μL of culture media and incubated overnight at 37 °C/5% CO₂. Lysate generated from haNK cells was added to the target cells 24 hrs after plating, and incubated for 3 hrs at 37 °C/5% CO₂. haNK cells were used because they can be expanded in culture without the presence of IL-2 in the culture medium. Once the co-incubation was complete, the plate was briefly centrifuged at 500 g for 2 min to collect the cells and remove the haNK cell lysate. After washing the cells in PBS, 200 μL of culture media is added to each well, and the cells are monitored for 5 days using the Incucyte® S3 Live-Cell Analysis System. Images were collected every 2 hrs. The effect of target cell proliferation was measured by identifying and evaluating the number of live cells present in each well expressing a nuclear-localized mKate2 fluorescence protein.

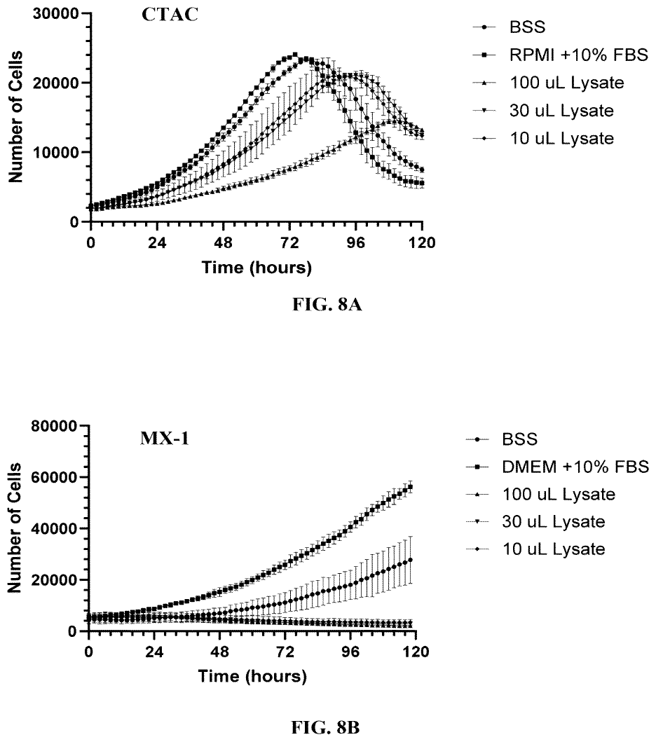

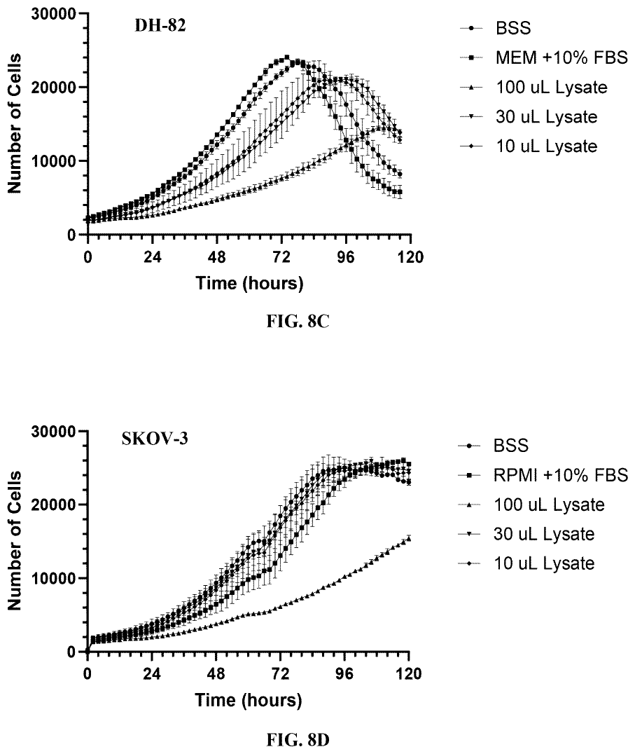

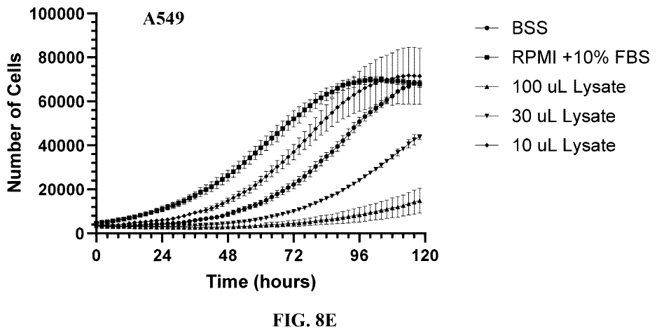

For each of the target cell lines (CTAC, MX-1, DH-82, SKOV-3, and A549), the total number of live cells is less compared to the controls when haNK cell lysate is added (FIG. 8A, FIG. 8B, FIG. 8C, FIG. 8D, and FIG. 8E). For example, at 80 hrs post lysate incubation, the total number of live DH-82 cells was about 19,000 when incubated with 10 μL of haNK cell lysate, while the total number of live DH-82 cells was about 10,000 when incubated with 100 μL of haNK cell lysate (FIG. 8C). A similar trend can be seen for CTAC cells (FIG. 8A), MX-1 cells (FIG. 8B), SKOV-3 cells (FIG. 8D), and A549 cells (FIG. 8E). Thus, haNK cell lysate can stop cell proliferation of CTAC, MX-1, DH-82, SKOV-3, and A549 target cell lines.

### Experiment 2: Lysate from haNK Cells is Cytotoxic and Anti-Proliferative to Malignant Human and Canine Cell Lines (Analysis of Lysate by Incucyte® Live-Cell Assay)

The NucLight™ Red-labeled human (SKOV-3, SKBR-3) and canine cancer cell lines (CTAC, OSCA-40), the cells were plated in 96-well plates at a seeding density of 1,000-2,000 cells per well in 100 μL of their respective culture media and incubated at 37 °C in a 5% CO₂ humidified incubator. After 24 hrs, culture media was removed, and 200 L/well of either culture media, BSS buffer, or lysate was added to the target cells. The plates were incubated for 15 min in a 37 °C/5% CO₂ humidified incubator. After co-incubation, the plates were briefly centrifuged at 500×g for 2 min, supernatants were discarded and cells were washed once in 1× D-PBS. Finally, 200 μL of culture media containing Cytotox green reagent (Sartorius) was added to each well to identify dead/dying cells. Plates were placed inside an Incucyte® S3 Live-Cell Imaging System device (Essen BioScience, Ann Harbor, MI), where four images per well were taken with a 20× objective every 2 hours over 72 hours in phase, green and red channels. Objects were counted by using the Incucyte® Analysis Software (version 2020B), by masking cells and signals for NucLight™-Red (red fluorescence) and/or Cytotox green (green fluorescence). Cell proliferation was quantified by counting the number of red fluorescent objects over the course of the assay and expressed as cell population doubling time for intervals of 6 hours, calculated using the formula ((t2−t1)×log(2))/(log(cell count at t2)−log(cell count at t1)), where (t2−t1) equals 6 hours. The number of live cells after lysate exposure was calculated as a percentage of dead/dying objects over total object count. All statistical analyses were performed using GraphPad Prism (Version 10.1.1) software. Results for cellular growth rates in the Incucyte® assays are expressed as average cell population doubling time±standard deviation (SD).

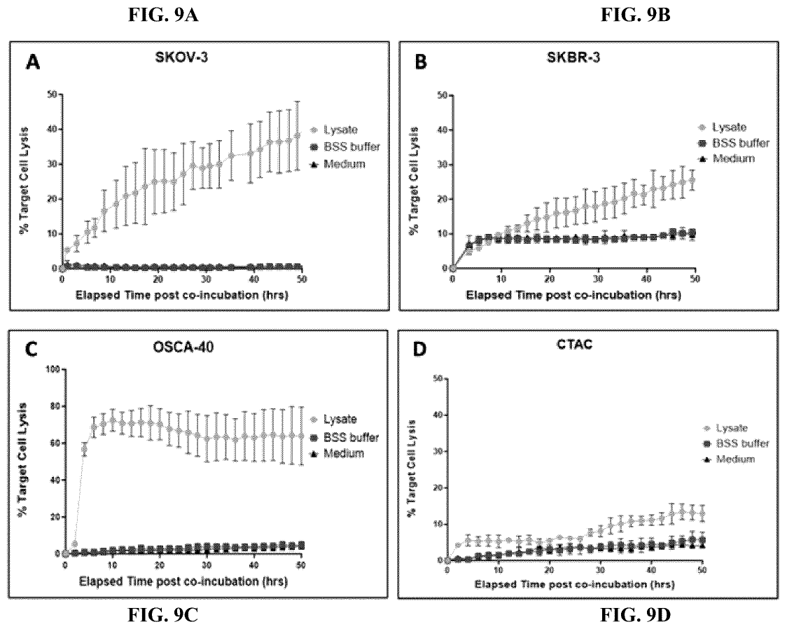

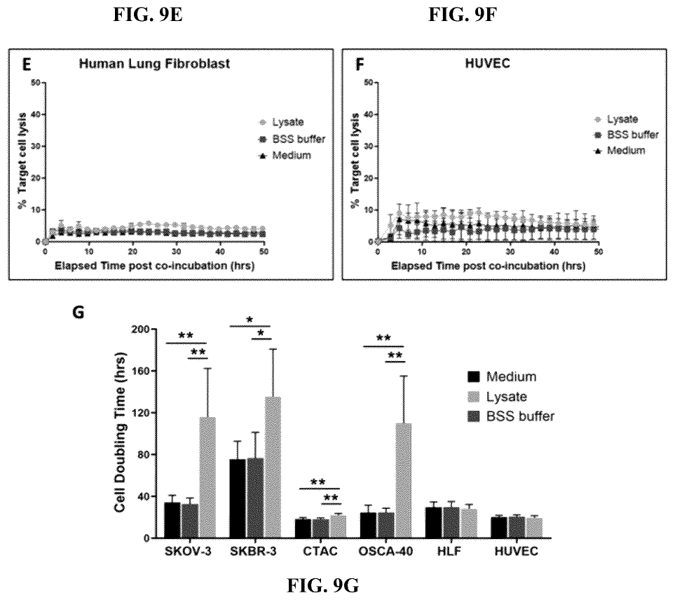

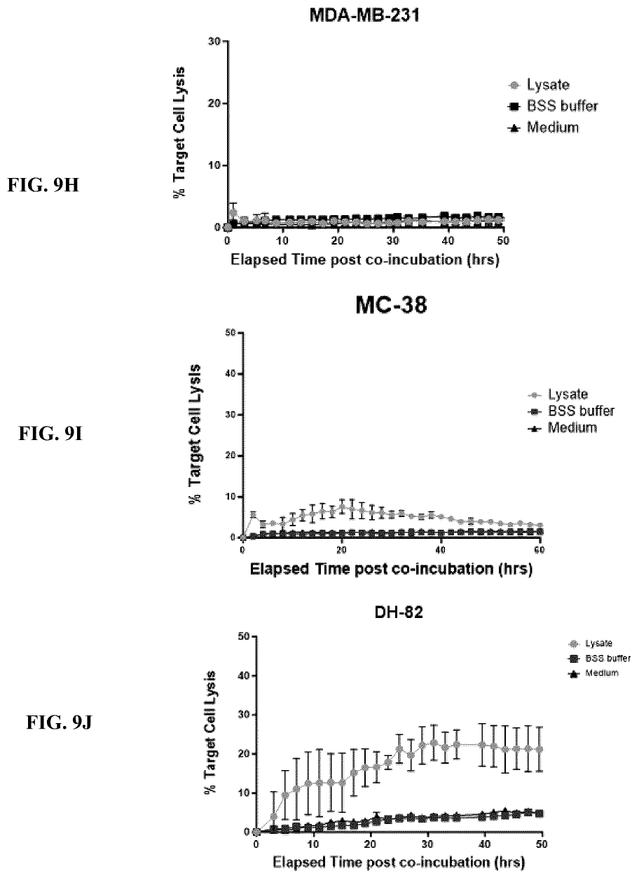

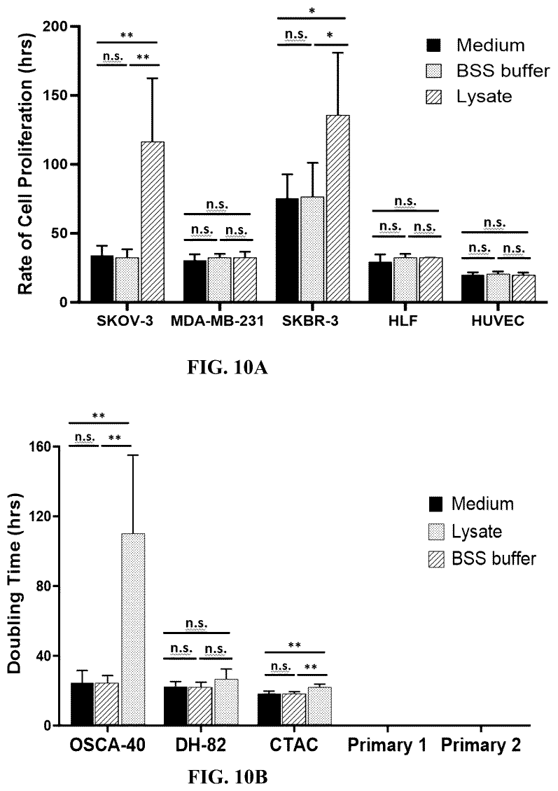

The Incuyte® S3 device allows for real time live cell analysis of target cells over several days. The results demonstrate that after only a short 15 minutes co-incubation with cancer targets, haNK lysate induces lysis of human SKOV-3 (FIG. 9A) and SKBR-3 (FIG. 9B), as well as of canine OSCA-40 (FIG. 9C) and CTAC (FIG. 9D), and DH-82 (macrophage-like cell derived from canine malignant histiocytosis, FIG. 9J) cancer cell lines. In contrast, haNK lysate did not appear to cause significant cell lysis in MDA-MB-231 (a highly aggressive, triple negative, breast cancer cell line, FIG. 9H) and MC-38 (murine colon adenocarcinoma cell line, FIG. 9I) cancer cell lines. Although both SKBR-3 and MDA-MB-231 are invasive breast cancer cells that express HER2, the haNK lysate had some cytotoxic effect on SKBR-3 (FIG. 9B) but not on MDA-MB-231 (FIG. 9H). For control purposes, primary human lung fibroblasts (FIG. 9E) and endothelial vein cells (HUVEC, FIG. 9F) were tested with haNK lysate under the same conditions and no cytotoxic effect was noted. The short co-incubation with lysate also induced significant cytostatic effect on the SKOV-3, SKBR-3, and OSCA-40 cancer cell lines, as evidenced by a longer cell population doubling time (FIG. 9G, FIG. 10A, and FIG. 10B). haNK cell lysate did not affect the population doubling time of the normal primary cell controls.

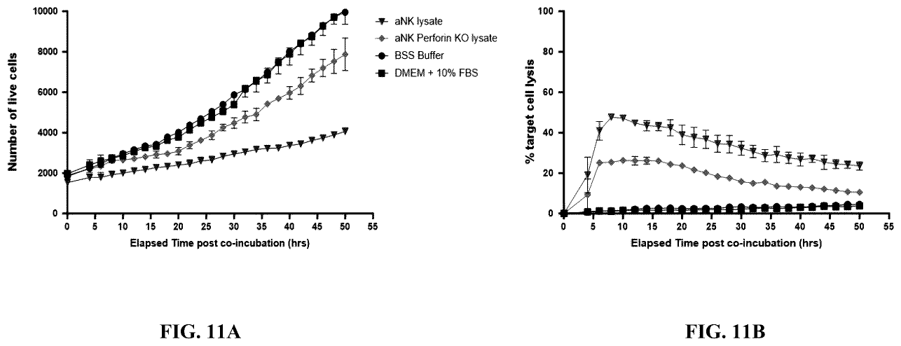

Cytotoxicity of aNK lysate was compared to the cytotoxicity of lysate from an aNK variant with a perforin knockout. As shown in FIG. 11A and FIG. 111B, deletion of perforin reduced aNK lysate cytotoxicity. While perforin could play a role NK-cell related cytoxicity, the results indicate that other players are likely involved.

### Experiment 3: Cryopreservation does not Affect the Biological Activity of NK-92 and haNK Cellular Lysate

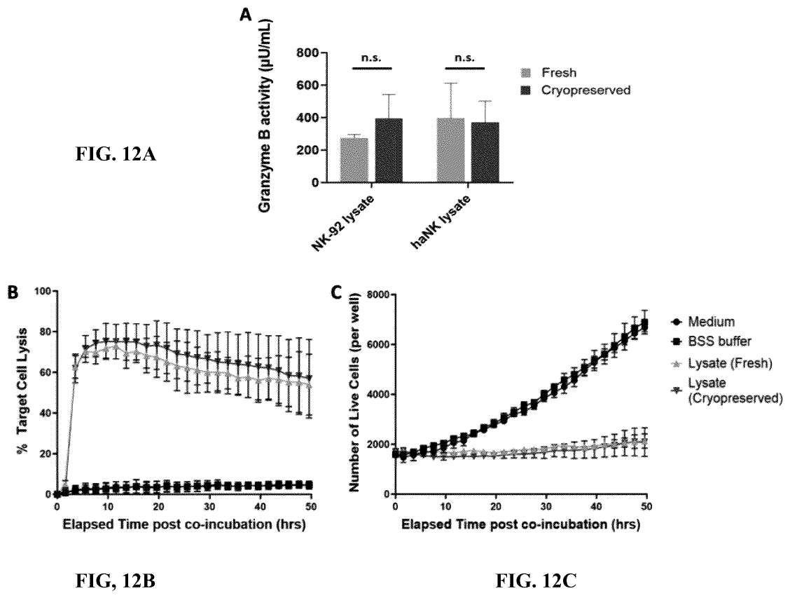

Because it is likely that NK lysate used clinically will have undergone previous cryopreservation, it was thus important to confirm that NK lysate activity would not be diminished by cryopreservation and subsequent thawing. As shown in FIG. 12A, cryopreservation/thawing of NK-92 or haNK lysate does not affect the activity of Granzyme B, a granzyme with key anti-tumor activity. As observed using the Incucyte® device, it was also confirmed that fresh and cryopreserved/thawed haNK lysate displayed similar cytotoxicity and anti-proliferative effects against canine OSCA-40 cancer cells (FIG. 12B and FIG. 12C, respectively).

### Example 3: Intratumoral Injection of NK Cell Lysate

Assessment of haNK Lysate Effects in Murine Tumor Studies

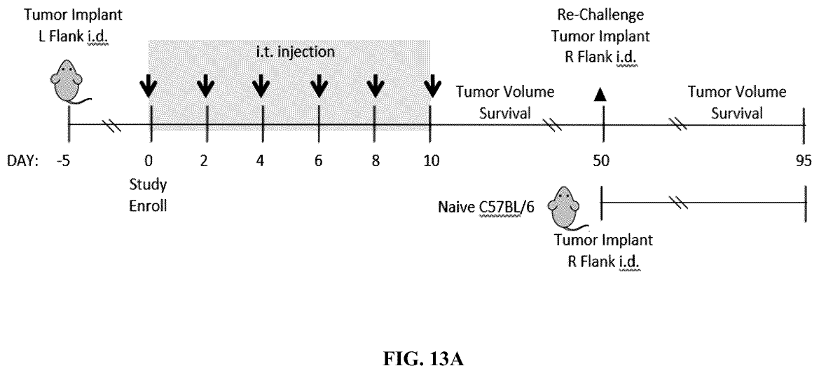

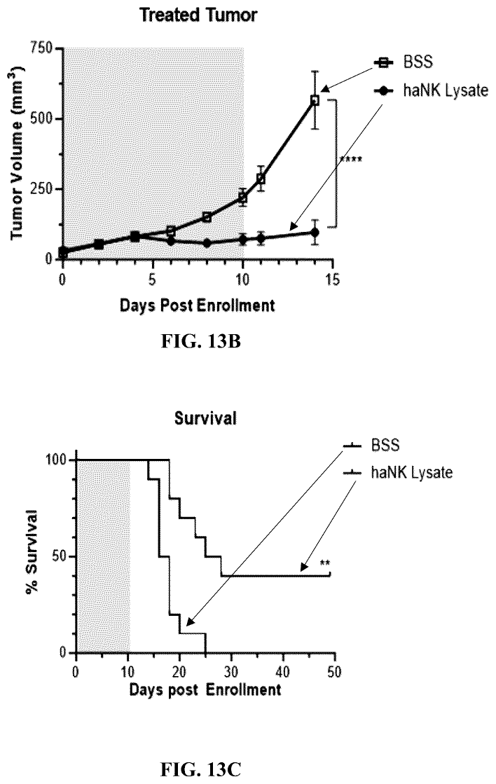

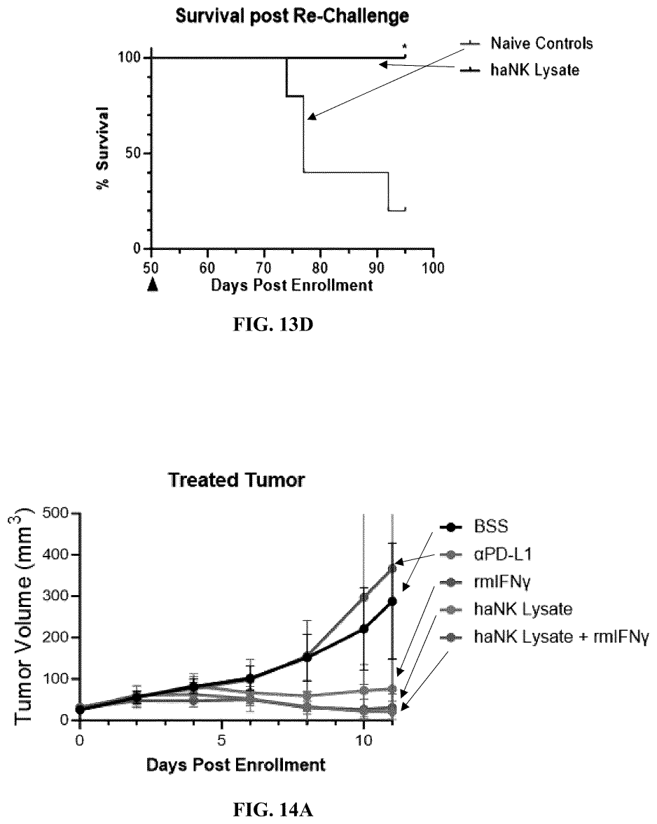

Female C57BL/6J mice were purchased from the Jackson Laboratory and were 7-8 weeks old at the start of experiments. Mouse studies were performed in accordance with the Guide for the Care and Use of Laboratory Animals approved by the local Institutional Animal Care and Use Committee. The study design discussed in this Example is as shown in FIG. 13A. Mice were anesthetized with isoflurane, their flank shaved, and were injected intra-dermally (i.d.) in the left hind flank with 3×10⁵ MC-38 tumor cells (in Dulbecco's phosphate buffered saline, DPBS) five days before study group enrollment. On the day of group enrollment (Day 0) mice with measurable tumors (volume range 20.1-44 mm³) were randomly enrolled into treatment groups (n=10/group). Following implantation, mice were monitored daily, and when the tumors reach a predetermined size, mice are randomly enrolled into treatment groups. The enrollment day was defined as Day 0. Tumors were measured 2-3 times per week using digital calipers by two opposing dimensions (a, b; wherein b is the smaller dimension) and tumor volume was calculated according to the formula V=(a×b²)/2. Mice were euthanized when tumor volume reached ≥1000 mm³ or exhibited non-resolving tumor ulceration.

In general, mice receive a once-daily intratumoral injection on Days 0, 2, 4, 6, 8, and 10. Prior to injection, mice were anaesthetized with isoflurane and 50 μL total volume (including NK cell lysate) was injected into the center of the tumor. Each injection was 50 μL bolus of BSS or haNK lysate at the equivalent of 5×10⁶ cells/50 μL dose. Mice that exhibited complete clearance of the primary tumor were re-challenged on Day 50 with 3×10⁵ MC-38 tumor cells i.d. on the right hind flank. At the time of re-challenge, age-matched naïve controls (n=5) were also implanted with MC-38 tumors on the right hind flank as a control for tumor implantation and growth. All surviving mice were euthanized at the termination of the study on Day 95. Results of primary tumor treatment are representative of two independent experiments. For combinatorial immunotherapy, the therapeutic reagents were admixed and administered in a single injection.

Over the course of the study, behavior is monitored daily and tumors are measured using digital calipers every 2-3 days. Two opposing tumor dimensions (a and b) are measured, and tumor volume is calculated according to the formula: volume=(a2×b)/2, where a is the shorter dimension. Mice are euthanized once a predetermined tumor volume is reached, or upon observation of adverse clinical signs.

All statistical analyses were performed using GraphPad Prism (Version 10.1.1) software. For in vivo tumor growth, unpaired t-test of growth rate as determined by exponential (Malthusian) growth were performed. Kaplan-Meier survival statistics were performed using Log-rank (Mantel-Cox) test relative to BSS controls or Naïve Controls. Statistical significance is indicated *p≤0.05, **p≤0.01, ***p≤0.001.

### Experiment 1: Intra-Tumor Injection of haNK Lysate Inhibits Tumor Growth, Extends Survival, and Protects Against Re-Challenge in Immunocompetent Mice

Cellular lysate from haNK cells was administered intra-tumor (i.t.) to immunocompetent female C57BL/6J mice bearing MC-38 tumors (FIG. 13A). Five days after intradermal tumor implantation, mice with measurable tumors were randomly assigned to the indicated treatment groups and received six i.t. injections of BSS or haNK lysate, administered once daily every two days starting on the day of enrollment.

All mice that received i.t. haNK lysate had significantly reduced tumor growth (FIG. 13B) relative to BSS controls with 40% of animals exhibiting complete tumor clearance. Mice that received haNK lysate also had prolonged survival as compared to BSS control (FIG. 13C).

To evaluate durability of the anti-tumor response, mice that exhibited complete eradication of the primary treated tumor (n=4) were re-challenged on Day 50 with the same MC-38 tumor line on the opposite flank along with a group of age-matched naïve controls (n=5). While tumors did develop in 4 of 5 naïve controls, none of the re-challenged mice developed tumors and all survived to the end of study on Day 95 (FIG. 13D).

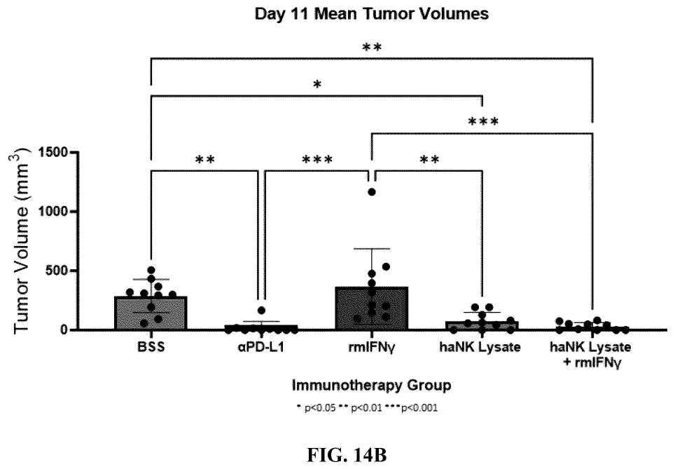

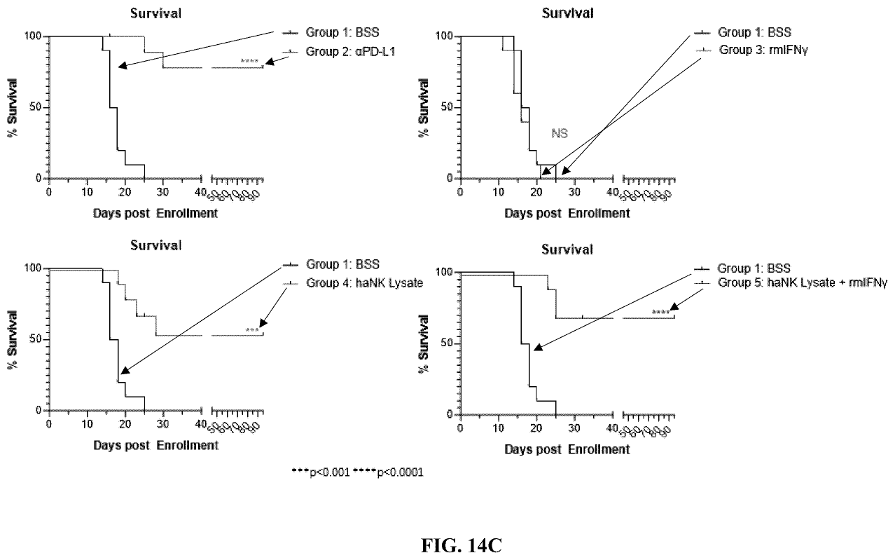

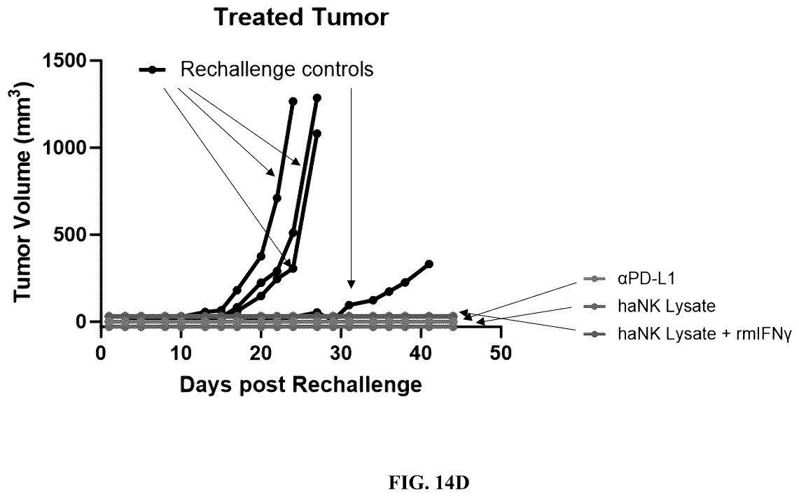

The experiments were repeated with haNK lysate supplemented with IFN-γ. Human IFN-γ does not react in mice. Thus, recombinant murine IFN-γ (rmIFN-γ) was added to the haNK lysate and mice treated with human IFN-γ or BSS buffer were used as negative controls. Mice treated with anti-PD-L1 antibody were used as control. As shown in FIG. 14A, FIG. 14B, and FIG. 14 C, mice treated with haNK lysate with and without rmIFN-γ resulted had decreased tumor volume and improved survival rates. Some mice treated with haNK lysate with rmIFN-γ (6 out of 10 treated mice) or with haNK lysate without rmIFN-γ (4 out of 10 treated mice) had no observable tumor volume left on the last day of the study (i.e., day 11), which indicated that the mice were cured of the tumor (FIG. 14A-14C). For comparison, in the positive control group that was treated with anti-PD-L1 antibody, 6 out of 10 mice (FIG. 14A-14C) had cleared tumors. Mice treated with rmIFN-γ alone had visibly the largest and most inflamed (i.e., red) tumors. The addition of haNK lysate to rmIFN-γ seemed to temper the inflammatory effect of IFN-γ.

On day 50, mice were re-challenged on the flank opposite (i.e., without the first tumor) with the same dose of MC-38 cells as the initial i.d. administration of MC-38 cells. Six age-matched mice did not receive treatment (rechallenge controls in FIG. 14D) were inoculated at the same time. Mice that were previously cured of tumors (FIG. 14A-14C) due to treatment with haNK lysate, haNK lysate with rmIFN-γ, or with the anti-PD-L1 antibody control did have any tumors grow on this opposite flank (FIG. 14D). In contrast, 4 out of the 5 untreated mice grew tumors. One rechallenge control mouse was removed from the study due to excessive grooming.

In summary, the results demonstrate that haNK lysate with or without rmIFN-γ can attenuate tumor growth in vivo, increase mouse survival, and provide a protective effect against future tumor growth.

### Example 4: Intratumoral Injection of NK Cell Lysate with IL-16 Knockout

Prior to NK cell lysis as described above, the IL-16 gene can be knocked out of an NK cell by known methods. After lysis, the NK cell lysate with an IL-16 knockout can be compared to an NK cell lysate without an IL-16 knockout. Such comparison can be carried out by intratumoral injection, as described above, of each lysate into the same type of tumor. For example, the NK cell lysate without an IL-16 knockout can be injected into at least one bladder cancer tumor, while the NK cell lysate can be injected into at least one separate bladder cancer tumor, under the same or similar conditions. Tumor variables, such as tumor cell death, tumor cell proliferation, and tumor reoccurrence can be compared between the two NK cell lysates.

### Discussion

The following Discussion relates to the Examples above. The NK-92 cell line—isolated in 1992 from a patient with lymphoma—has been extensively used by investigators worldwide for studies to further define the characteristics of NK cells as the NK-92 cell line can be considered a suitable surrogate for blood NK cells [References 12, 13]. NK-92 cells have been genetically engineered with IL-2 to make their ex vivo growth IL-2 independent [References 19, 26]. A variant expressing the high affinity FcγRIIIa-receptor (haNK cells) can bind antibodies and execute ADCC [Reference 19]. The transgene construct for haNK cells also includes an erIL-2 sequence to allow haNK cells to grow and expand independently of exogeneous IL-2. Various CAR-expressing modifications of NK-92 have been generated: taNK [References 20, 21] and t-haNK [References 21-26]. See, also, U.S. Pat. No. 11,643,452. Patients with advanced cancers have been treated with NK-92, haNK, taNK and t-haNK cells with some encouraging responses and minimal side effects [discussed in References 13, 25]. The Examples above discuss the data related to a novel NK-92/haNK cell-derived product—i.e., a cellular lysate—that is generated by repeat freeze-thaw cycles.

Lysates from NK-92 cells and haNK cells were tested side by side—showing essentially equivalent levels for perforin/granzyme expression and cytokine concentration (FIG. 4B and FIG. 5C). Since haNK cells do not require exogenous IL-2 for expansion, they would be preferred for any clinical use over unmodified NK-92 cells. Hence, in vitro and in vivo experiments were performed (as discussed in the Examples above) with haNK lysate. Importantly, the isolation process for lysate preparation was shown to yield a product with significant cytolytic and anti-proliferative activity. In the clinical setting, for logistical reasons, the use of previously cryopreserved lysate would be preferred over fresh material, for example fresh cells or freshly prepared cell lysate. As discussed in the Examples above, repeated freezing and thawing does not affect the activity of Granzyme B and the cytotoxic and anti-proliferative activity of the lysate.

When considering potential clinical applications of NK-92/haNK lysate, intra-tumor and peri-tumor injections are possible options. The presence of perforin and the broad spectrum of granzymes in the lysate should allow for entry of cytolytic enzymes into the tumor cells causing their lysis. This direct anti-tumor activity should be facilitated by the immune augmenting activity of TNF-0, IL-2, IL-16 and IFN-7. Evidence of this mechanism is provided by the murine data discussed in the Examples demonstrating that intra-tumor injection of haNK lysate into MC-38 tumors of immunocompetent mice significantly reduced tumor growth and prolonged their survival. Importantly, this anti-tumor response was durable, as mice that had cleared the primary treated tumor did not develop tumors upon re-challenge. The presence of IL-16 in the lysate may support such a vaccine effect as the cytokine attracts CD4 T-cells [Reference 27] and also stimulates macrophages to secrete IL-1α, IL-6 and IL-15 creating a proinflammatory environment [Reference 28].

Such a vaccine-like effect with NK-92 (whole cell) administration had been observed before. When (murine) CD19-CAR engineered NK-92 were injected into A20 lymphoma of Balb/c mice, complete tumor elimination occurred in the majority of mice [Reference 23]. When those mice were re-challenged several weeks later with A20 lymphoma cells on the opposite site, no tumor growth/recurrence occurred. Similar data were presented by Wels et al. [Reference 21] who injected Her-2 CAR expressing NK-92 cells into immunocompetent mice with glioblastoma. Those mice that achieved a full remission did not develop cancer upon rechallenge.

Systemic cellular therapy of solid tumors, even with targeted CAR-T cells, has so far not achieved the same anti-tumor responses as seen for hematological malignancies [References 29-31]. This may be due to the fact that a whole cell product faces the challenge of sufficiently infiltrating the solid tumor tissue due to its fibrous architecture in addition to the immunosuppressive tumor microenvironment [Reference 32]. Park et al. [Reference 33] recently described the negative impact of the peritumor glycocalyx on the cytotoxic function of NK-92 which, however, could be overcome by NK-92 cells engineered to degrade glycocalyx.

The testing of canine tumor cell lines here was deliberate, as cancer in dogs represents another possible indication for the use of haNK lysate. This lysate was cytotoxic and cytostatic to canine cancer cells and would be more feasible than attempting to prepare cell lysate from NK cells derived from canine blood, as canine NK cells are not well described or functionally characterized [Reference 34]. Although expansion of canine peripheral blood mononuclear cells on an adherent layer may provide some cytotoxic cells, there is substantial donor to donor variability [Reference 35].

The observation that NK-92 lysate is cytolytic/cytostatic to canine cancer cells correlates with the observation that cross-reactivity can occur between about 80% of human and canine proteins [Reference 36]. In fact, treatment of canines with human cytokines like IL-2 or IL-15 has shown anti-tumor effects [References 37, 38]. Although there is concern that the xenogeneic proteins in the lysate may cause an immune response in the recipient, the strength of such an immune response is not known. Previous studies have shown that injection of the human TALL-104 cell line into dogs with cancer did not induce an immediate rejection [Reference 39]. Moreover, injection of NK-92 cells and as demonstrated herein, NK-92 cell lysates, into immunocompetent mice elicited a long-lasting vaccine-like response [References 21,23].

The logistics of manufacturing clinical grade NK-92 lysate are relatively straightforward. NK-92/haNK cells can be expanded without the need for an adherent layer from 10⁶ to 10⁸ cells within 8-10 days. Extrapolating these numbers to a larger scale: a 50 liter tank generating about 25 billion NK-92/haNK cells could yield about 250 (1 ml) lysate doses. Because cryopreservation does not affect the activity of the NK-92/haNK cellular lysates, it is feasible to have the product readily available at the point of patient treatment.

In summary, cellular lysate from NK-92/haNK cells contains significant concentrations of perforin, granzymes, and immune-active cytokines/chemokines with a cytotoxic and growth inhibitory effect on both human and canine cancer cells. Intra-tumor injection of the lysate elicits a robust and durable anti-tumor response, significantly reducing tumor growth and extending survival of immunocompetent mice as well as preventing growth of re-challenge tumors. Hence, cellular lysate generated from NK-92/haNK cell holds potential as a therapeutic modality for certain human and canine cancers.

### REFERENCES

The following references relate to the Examples above and are hereby incorporated by reference.
1 da Fonseca L, Santos G S, Huber S C, Setti T M, Setti T, Lana J F. Human platelet lysate—A potent (and overlooked) orthobiologic. J Clin Orthop Trauma. 2021; 21:101534. https://doi.org/10.1016/j.jcot.2021.101534. PMID: 34386346; PMCID: PMC833933
2 Meftahpour V, Malekghasemi S, Baghbanzadeh A, et al. Platelet lysate: a promising candidate in regenerative medicine. Regen Med. 2021; 16(1):71-85. https://doi.org/10.2217/rme-2020-0065. Epub 2021 Feb. 5. PMID: 33543999.
3 Oeller M, Laner-Plamberger S, Krisch L, Rohde E, Strunk D, Schallmoser K. Human Platelet Lysate for Good Manufacturing Practice-Compliant Cell Production. Int J Mol Sci. 2021; 22(10):5178. https://doi.org/10.3390/ijms22105178. PMID: 34068404; PMCID: PMC8153614.
4 Zamani M, Yaghoubi Y, Movassaghpour A, et al. Novel therapeutic approaches in utilizing platelet lysate in regenerative medicine: Are we ready for clinical use?. J Cell Physiol. 2019; 234(10):17172-17186. https://doi.org/10.1002/jcp.28496. PMID: 30912141.
5 González FE, Gleisner A, Falcón-Beas F, Osorio F, López MN, Salazar-Onfray F. Tumor cell lysates as immunogenic sources for cancer vaccine design. Hum Vaccin Immunother. 2014; 10(11):3261-3269. https://doi.org/10.4161/21645515.2014.982996. PMID: 25625929; PMCID: PMC4514089.
6 Schnurr M, Galambos P, Scholz C, et al. Tumor cell lysate-pulsed human dendritic cells induce a T-cell response against pancreatic carcinoma cells: an in vitro model for the assessment of tumor vaccines. Cancer Res. 2001; 61(17):6445-6450. PMID: 11522639
7 Suárez N, Ferrara F, Rial A, Dee V, Chabalgoity J A. Bacterial Lysates as Immunotherapies for Respiratory Infections: Methods of Preparation. Front Bioeng Biotechnol. 2020; 8:545. https://doi.org/10.3389/fbioe.2020.00545. PMID: 32582669; PMCID: PMC7289947.
8 Rahman M M, Grice I D, Ulett G C, Wei M Q. Advances in Bacterial Lysate Immunotherapy for Infectious Diseases and Cancer. J Immunol Res. 2024; 2024:4312908. https://doi.org/10.1155/2024/4312908. PMID: 38962577; PMCID: PMC11221958.
9 Duarte Rojas J M, Restrepo Mnnera L M, Estrada Mira S. Comparison between Platelet Lysate, Platelet Lysate Serum, and Fetal Bovine Serum as Supplements for Cell Culture, Expansion, and Cryopreservation. Biomedicines. 2024; 12(1):140. https://doi.org/10.3390/biomedicines12010140. PMID: 38255245; PMCID: PMC10813006.
10 Shen S, Shen Z, Wang C, et al. Effects of lysate/tissue storage at −80 °C on subsequently extracted EVs of epithelial ovarian cancer tissue origins. iScience. 2023; 26(4):106521. https://doi.org/10.1016/j.isci.2023.106521. PMID: 37123245; PMCID: PMC10139970.
11 Maia A, Tarannum M, Lerias J R, et al. Building a Better Defense: Expanding and Improving Natural Killer Cells for Adoptive Cell Therapy. Cells. 2024; 13(5):451. https://doi.org/10.3390/cells13050451. PMID: 38474415; PMCID: PMC10930942.
12 Gong J H, Maki G, Klingemann H G. Characterization of a human cell line (NK-92) with phenotypical and functional characteristics of activated natural killer cells. Leukemia. 1994; 8(4):652-658. PMID: 8152260.
13 Klingemann H. The NK-92 cell line-30 years later: its impact on natural killer cell research and treatment of cancer. Cytotherapy. 2023; 25(5):451-457. https://doi.org/10.1016/j.jcyt.2022.12.003. Epub 2023 Jan. 6. PMID: 36610812.
14 Arai S, Meagher R, Swearingen M, et al. Infusion of the allogeneic cell line NK-92 in patients with advanced renal cell cancer or melanoma: a phase I trial. Cytotherapy. 2008; 10(6):625-632. https://doi.org/10.1080/14653240802301872. PMID: 18836917.
15 Williams B A, Law A D, Routy B, et al. A phase I trial of NK-92 cells for refractory hematological malignancies relapsing after autologous hematopoietic cell transplantation shows safety and evidence of efficacy. Oncotarget. 2017; 8(51):89256-89268. https://doi.org/10.18632/oncotarget.19204. PMID: 29179517; PMCID: PMC5687687.
16 Boyiadzis M, Agha M, Redner R L, et al. Phase 1 clinical trial of adoptive immunotherapy using “off-the-shelf” activated natural killer cells in patients with refractory and relapsed acute myeloid leukemia. Cytotherapy. 2017; 19(10):1225-1232. https://doi.org/10.1016/j.jcyt.2017.07.008. Epub 2017 Aug. 30. PMID: 28864289.
17 Tonn T, Schwabe D, Klingemann H G, et al. Treatment of patients with advanced cancer with the natural killer cell line NK-92. Cytotherapy. 2013; 15(12):1563-1570. https://doi.org/10.1016/j.jcyt.2013.06.017. Epub 2013 Oct. 1. PMID: 24094496.
18 Bhatia S. B. M., Zhang H., Lee T. Adoptive cellular therapy (act) with allogeneic activated natural killer (aNK) cells in patients with advanced Merkel cell carcinoma (MCC): preliminary results of a phase 2 trial. Society for Immunotherapy of Cancer Annual Meeting, National Harbor, MD. 2016.
19 Jochems C, Hodge J W, Fantini M, et al. An NK cell line (haNK) expressing high levels of granzyme and engineered to express the high affinity CD16 allele. Oncotarget. 2016; 7(52):86359-86373. https://doi.org/10.18632/oncotarget.13411. PMID: 27861156; PMCID: PMC5341330.
20 Burger M C, Forster M T, Romanski A, et al. Intracranial injection of natural killer cells engineered with a HER2-targeted chimeric antigen receptor in patients with recurrent glioblastoma. Neuro Oncol. 2023; 25(11):2058-2071. https://doi.org/10.1093/neuonc/noad087. PMID: 37148198; PMCID: PMC10628939.
21 Schönfeld K, Sahm C, Zhang C, et al. Selective inhibition of tumor growth by clonal NK cells expressing an ErbB2/HER2-specific chimeric antigen receptor. Mol Ther. 2015; 23(2):330-338. https://doi.org/10.1038/mt.2014.219. PMID: 25373520; PMCID: PMC4445620.
22 Fabian K P, Padget M R, Donahue R N, et al. PD-L1 targeting high-affinity NK (t-haNK) cells induce direct antitumor effects and target suppressive MDSC populations. J Immunother Cancer. 2020; 8(1):e000450. https://doi.org/10.1136/jitc-2019-000450. PMID: 32439799; PMCID: PMC7247398.
23 Boissel L., Klingemann H., Khan J., Soon-Shiong P. Intra-tumor injection of CAR-engineered NK cells induces tumor regression and protection against tumor-rechallenge. Blood. 2016; 128(22): 466. https://doi.org/10.1182/blood.V128.22.466.466.
24 Klingemann H. Are natural killer cells superior CAR drivers?. Oncoimmunology. 2014; 3:e28147. https://doi.org/10.4161/onci.28147. PMID: 25340009; PMCID: PMC4203506.
25 Zhang C, Oberoi P, Oelsner S, et al. Chimeric Antigen Receptor-Engineered NK-92 Cells: An Off-the-Shelf Cellular Therapeutic for Targeted Elimination of Cancer Cells and Induction of Protective Antitumor Immunity. Front Immunol. 2017; 8:533. https://doi.org/10.3389/fimmu.2017.00533. PMID: 28572802; PMCID: PMC5435757.
26 Tam Y K, Maki G, Miyagawa B, Hennemann B, Tonn T, Klingemann H G. Characterization of genetically altered, interleukin 2-independent natural killer cell lines suitable for adoptive cellular immunotherapy. Hum Gene Ther. 1999; 10(8):1359-1373. https://doi.org/10.1089/10430349950018030. PMID: 10365666.
27 Liu Y, Cruikshank W W, O'Loughlin T, O'Reilly P, Center D M, Kornfeld H. Identification of a CD4 domain required for interleukin-16 binding and lymphocyte activation. J Biol Chem. 1999; 274(33):23387-23395. https://doi.org/10.1074/jbc.274.33.23387. PMID: 10438516.
28 Mathy N L, Scheuer W, Lanzendörfer M, et al. Interleukin-16 stimulates the expression and production of pro-inflammatory cytokines by human monocytes. Immunology. 2000; 100(1):63-69. https://doi.org/10.1046/j.1365-2567.2000.00997.x. PMID: 10809960; PMCID: PMC2326980.
29 St Martin Y, Franz J K, Agha M E, Lazarus H M. Failure of CAR-T cell therapy in relapsed and refractory large cell lymphoma and multiple myeloma: An urgent unmet need. Blood Rev. 2023; 60:101095. https://doi.org/10.1016/j.blre.2023.101095. Epub 2023 Apr. 29. PMID: 37173224.
30 Uslu, U., June, C. H. Beyond the blood: expanding CAR T cell therapy to solid tumors. Nat Biotechnol. 2024. https://doi.org/10.1038/s41587-024-02446-2
31 Brudno J N, Kochenderfer J N. Toxicities of chimeric antigen receptor T cells: recognition and management. Blood. 2016; 127(26):3321-3330. https://doi.org/10.1182/blood-2016-04-703751. Epub 2016 May 20. PMID: 27207799; PMCID: PMC4929924.
32 Labani-Motlagh A, Ashja-Mahdavi M, Loskog A. The Tumor Microenvironment: A Milieu Hindering and Obstructing Antitumor Immune Responses. Front Immunol. 2020; 11:940. https://doi.org/10.3389/fimmu.2020.00940. PMID: 32499786; PMCID: PMC7243284.
33 Park S, Colville M J, Paek J H, et al. Immunoengineering can overcome the glycocalyx armour of cancer cells. Nat Mater. 2024; 23(3):429-438. https://doi.org/10.1038/s41563-024-01808-0. Epub 2024 Feb. 15. PMID: 38361041; PMCID: PMC11471287.
34 Park S, Paek J H, Colville M J, et al. Leucine zipper-based SAIM imaging identifies therapeutic agents to disrupt the cancer cell glycocalyx for enhanced immunotherapy. Preprint. bioRxiv. 2024; 2024.12.05.627089. https://doi.org/10.1101/2024.12.05.627089. PMID: 39677754; PMCID: PMC11643053.
35 Gingrich A A, Modiano J F, Canter R J. Characterization and Potential Applications of Dog Natural Killer Cells in Cancer Immunotherapy. J Clin Med. 2019; 8(11):1802. https://doi.org/10.3390/jcm8111802. PMID: 31717876; PMCID: PMC6912828.
36 Razmara A M, Farley L E, Harris R M, et al. Preclinical evaluation and first-in-dog clinical trials of PBMC-expanded natural killer cells for adoptive immunotherapy in dogs with cancer. J Immunother Cancer. 2024; 12(4):e007963. https://doi.org/10.1136/jitc-2023-007963. PMID: 38631708; PMCID: PMC11029326.
37 Klingemann H. Can human immuno-therapeutics be considered for cancer treatment of dogs. Front Vet Sciences, in press
38 Helfand S C, Soergel S A, MacWilliams P S, Hank J A, Sondel P M. Clinical and immunological effects of human recombinant interleukin-2 given by repetitive weekly infusion to normal dogs. Cancer Immunol Immunother. 1994; 39(2):84-92. https://doi.org/10.1007/BF01525313. PMID: 8044833
39 Rebhun R B, York D, Cruz S M, et al. Inhaled recombinant human IL-15 in dogs with naturally occurring pulmonary metastases from osteosarcoma or melanoma: a phase 1 study of clinical activity and correlates of response. J Immunother Cancer. 2022; 10(6):e004493. https://doi.org/10.1136/jitc-2022-004493. PMID: 35680383; PMCID: PMC9174838.
40 Visonneau S, Cesano A, Jeglum K A, Santoli D. Adjuvant treatment of canine osteosarcoma with the human cytotoxic T-cell line TALL-104. Clin Cancer Res. 1999; 5(7):1868-1875. PMID: 10430094

1. A natural killer (NK) cell lysate comprising:
a salt solution;
a cytotoxic protein; and
a cytokine; wherein the cytokine is interleukin-16 (IL-16), preferably an active form of IL-16, and wherein the IL-16 is endogenously produced by NK cells.

2. The NK cell lysate of claim 1, wherein the NK cells are NK-92/aNK cells, haNK cells, t-haNK cells, primary NK cells, KHYG-1 cells, or a variant thereof.

3. The NK cell lysate of claim 1, comprising no detergent.

4. The NK cell lysate of claim 1, wherein the cytotoxic protein is granzyme A (GzmA), granzyme B (GzmB), granzyme H (GzmH), granzyme K (GzmK), granzyme M (GzmM), gasdermin D (GSDMD), perforin 1 (PRF1), granulysin (GNLY), or any combination thereof; and wherein the cytotoxic protein is endogenously or recombinantly produced by the NK cells prior to lysis, or the cytotoxic protein is exogenously added before or after the NK cells are lysed.

5.-6. (canceled)

7. The NK cell lysate of claim 1, further comprising one or more additional cytokines, an agonist derivative of the cytokine, or a fusion construct comprising the cytokine and/or agonist derivative of the cytokine, wherein
the cytokines are chosen from IL-1α, IL-1β, IL-2, IL-4, IL-5, IL-6, IL-7, IL-10, IL-12p40, IL-12p70, IL-13, IL-15, IL-17, IL-18, INF-γ, GM-CSF, TNF-α, TNF-β, VEGF, IL-8, Eotaxin-3, MCP-1, MCP-4, MDC, MIP-1α, MIP-1β, and TARC; and
the agonist derivatives of the cytokines are chosen from IL-1α, IL-1β, IL-2, IL-4, IL-5, IL-6, IL-7, IL-10, IL-12p40, IL-12p70, IL-13, IL-15, IL-17, IL-18, INF-γ, GM-CSF, TNF-α, TNF-β, VEGF, IL-8, Eotaxin-3, MCP-1, MCP-4, MDC, MIP-1α, MIP-1β, and TARC agonist derivatives;
wherein the one or more additional cytokines, an agonist derivative of the cytokine, or a fusion construct comprising the cytokine and/or agonist derivative of the cytokine is endogenously or recombinantly produced by the NK cells prior to lysis, or is exogenously added before or after the NK cells are lysed.

8.-9. (canceled)

10. The NK cell lysate of claim 1, further comprising a yeast lysate.

11.-12. (canceled)

13. The NK cell lysate of claim 1, wherein the NK cell lysate comprises at least about 2,000 pg/mL of IL-16.

14.-19. (canceled)

20. A composition comprising the NK cell lysate of claim 1.

21. The composition of claim 20, further comprising one or more NK cell exosomes derived from NK-92/aNK cells, haNK cells, t-haNK cells, primary NK cells, KHYG-1 cells, or a variant thereof.

22.-25. (canceled)

26. The composition of claim 21, further comprising one or more additional cytokines, agonist derivatives of the cytokines, or fusion constructs comprising the cytokines and/or agonist derivatives of the cytokines; wherein
the cytokines are chosen from IL-1α, IL-1β, IL-2, IL-4, IL-5, IL-6, IL-7, IL-10, IL-12p40, IL-12p70, IL-13, IL-15, IL-17, IL-18, INF-γ, GM-CSF, TNF-α, TNF-β, VEGF, IL-8, Eotaxin-3, MCP-1, MCP-4, MDC, MIP-1α, MIP-1β, and TARC; and
the agonist derivatives of the cytokines are chosen from IL-1α, IL-1β, IL-2, IL-4, IL-5, IL-6, IL-7, IL-10, IL-12p40, IL-12p70, IL-13, IL-15, IL-17, IL-18, INF-γ, GM-CSF, TNF-α, TNF-β, VEGF, IL-8, Eotaxin-3, MCP-1, MCP-4, MDC, MIP-1α, MIP-1β, and TARC agonist derivatives.

27.-33. (canceled)

34. A method of preparing the NK cell lysate of claim 1, comprising:
lysing the NK cells to produce a lysate,
wherein lysing the NK cells comprises one or more cycles of sonicating the NK cells or one or more cycles of freezing and thawing the NK cells, and
removing intact NK cells, NK cell debris, and NK cell genomic DNA from the lysate.

35. The method of claim 34, further comprising:
(i) substantially removing MHC class I and/or MHC class II molecules from the lysate; and/or
(ii) expanding the NK cells in a cell culture medium prior to lysing the NK cells; and the NK cells are expanded with IL-2, IL-12, IL-15, IL-18, or a combination thereof.

36. The method of claim 34, wherein:
(i) the NK cells are suspended in a salt solution prior to lysing the NK cells;
(ii) no detergent is added to the lysing step; and/or
(iii) the removing of intact NK cells, NK cell debris, and NK cell genomic DNA comprises centrifugation.

37.-42. (canceled)

43. A method of preparing a composition for use in the treatment of cancer, the method comprising:
expanding primary human NK cells in a cell culture comprising IL-15 or an agonist derivative thereof, IL-12 or an agonist derivative thereof, and IL-18 or an agonist derivative thereof, thereby producing memory-like cytokine enhanced NK cells (m-ceNK);
collecting exosomes from the supernatant of the m-ceNK cells;
lysing the m-ceNK cells to produce a lysate, wherein lysing the m-ceNK cells comprises one or more cycles of sonicating the m-ceNK cells or one or more cycles of freezing and thawing the m-ceNK cells;
removing intact NK cells, NK cell debris, and NK cell genomic DNA from the lysate; and
formulating an effective amount of the exosomes and an effective amount of the lysate for administration to a human subject in need thereof.

44. The method of claim 43, wherein:
(i) the m-ceNK cells are suspended in a salt solution prior to lysing the m-ceNK cells;
(ii) no detergent is added to the lysing step; and/or
(iii) the removing of intact NK cells, NK cell debris, and NK cell genomic DNA comprises centrifugation.

45.-48. (canceled)

49. A method for preventing and/or treating cancer; initiating, enhancing, or prolonging an anti-tumor response; or stimulating an immune response in a subject in need thereof, the method comprising administering to the subject an effective amount of the NK cell lysate of claim 1.

50.-54. (canceled)

55. The method of any one of claim 49, wherein the NK cell lysate or composition is administered by intravenous, intranasal, or intratumoral injection.

56.-59. (canceled)

60. A method for preventing and/or treating an infection in a subject in need thereof, the method comprising administering to the subject an effective amount of the NK cell lysate of claim 1.

61.-63. (canceled)

64. The method of claim 60, wherein the NK cell lysate or composition is administered by injection into an infected abscess.

65. (canceled)

66. A combination therapy for preventing and/or treating cancer in a subject in need thereof, wherein the combination therapy comprises administration of a first component comprising the NK cell lysate of claim 1, and administration of a second component comprising a therapeutically active agent chosen from a chemotherapeutic agent, a therapeutic agent, a targeted drug, and an immunomodulator.

67. The combination therapy of claim 66, wherein:
(i) the therapeutic agent comprises unlysed NK-92/aNK cells, haNK cells, t-haNK cells, primary NK cells, KHYG-1 cells, m-CENK cells, or a combination thereof; or
(ii) the immunomodulator comprises IL-12, IL-15, IL-15:IL-15Rα or a stabilized derivative thereof, or a yeast lysate; a tumor necrosis targeting (TNT) antibody; an immune checkpoint modulator; or a combination thereof.

68.-73. (canceled)

74. The combination therapy of claim 66, wherein the first component is administered via intratumoral injection or inhalation, and the second component is administered via intravenous administration.

75.-76. (canceled)

77. A method of producing IL-16 from NK cells, the method comprising:
lysing the NK cells to produce a lysate, wherein lysing the NK cells comprises one or more cycles of sonicating the NK cells or one or more cycles of freezing and thawing the NK cells, and
removing intact NK cells, NK cell debris, and NK cell genomic DNA from the lysate.

78. The method of claim 77, further comprising:
(i) substantially removing MHC class I and/or MHC class II molecules from the lysate;
(ii) isolating the IL-16 from the lysate;
(iii) suspending the NK cells in a salt solution prior to lysing the NK cells; and/or
(iv) expanding the NK cells in a cell culture medium; and wherein the NK cells are expanded with IL-2, IL-12, IL-15, IL-18, or a combination thereof.

79. The method of claim 77 wherein the NK cells are NK-92/aNK cells, haNK cells, t-haNK cells, primary NK cells, KHYG-1 cells, or a variant thereof; and/or the NK cells are not genetically engineered to express IL-16.

80.-88. (canceled)

89. The method of claim 77, wherein no detergent is added during lysis.

90. The method of claim 77, wherein the method produces at least about 2,000 pg/mL of IL-16, and/or an active form of IL-16.

91. (canceled)

92. A vaccine comprising the NK cell lysate of claim 1.


SEQUENCE LISTING (5 sequences)

SEQ ID NO: 1 - protein, 7 aa, synthetic construct
GSEKDEL

SEQ ID NO: 2 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 3 - DNA, 483 bp, synthetic construct
  source 1..483: mol_type = other DNA
atgtaccggatgcagctgctgagctgtatcgccctgtctctggccctcgtgaccaacagcgcccctaccagcagcagcaccaagaaaacccagctgcagctggaacatctgctgctggacctgcagatgatcctgaacggcatcaacaactacaagaaccccaagctgacccggatgctgaccttcaagttctacatgcccaagaaggccaccgaactgaaacatctgcagtgcctagaagaggaactgaagcccctggaagaagtgctgaacctggcccagagcaagaacttccacctgaggcccagggacctgatcagcaacatcaacgtgatcgtgctggaactgaaaggcagcgagacaaccttcatgtgcgagtacgccgacgagacagctaccatcgtggaatttctgaaccgatggatcaccttctgccagagcatcatcagcaccctgaccggctccgagaaggacgagctgtga

SEQ ID NO: 4 - protein, 160 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSEKDEL

SEQ ID NO: 5 - protein, 160 aa, synthetic construct; identical to SEQ ID NO: 4